SEQUENCE LISTING (16 sequences)

SEQ ID NO: 1 - protein, 5 aa, Artificial
  synthetic polypeptide
DYYIH

SEQ ID NO: 2 - protein, 17 aa, Artificial
  synthetic polypeptide
YINPNSGYTNYAQKFQG

SEQ ID NO: 3 - protein, 12 aa, Artificial
  synthetic polypeptide
YMWERVTGFFDF

SEQ ID NO: 4 - protein, 121 aa, Artificial
  Heavy Chain Variable Domain
QVQLVQSGAEVKKPGASVKLSCKASGYTFTDYYIHWVRQAPGQGLEWIGYINPNSGYTNYAQKFQGRATMTADKSINTAYVELSRLRSDDTAVYFCTRYMWERVTGFFDFWGQGTMVTVSS

SEQ ID NO: 5 - protein, 11 aa, Artificial
  synthetic polypeptide
LASEDISDDLA

SEQ ID NO: 6 - protein, 7 aa, Artificial
  synthetic polypeptide
TTSSLQS

SEQ ID NO: 7 - protein, 9 aa, Artificial
  synthetic polypeptide
QQTYKFPPT

SEQ ID NO: 8 - protein, 108 aa, Artificial
  Light Chain Variable Domain
DIQMTQSPSSVSASVGDRVTITCLASEDISDDLAWYQQKPGKAPKVLVYTTSSLQSGVPSRFSGSGSGTDFTLTISSLQPEDFATYFCQQTYKFPPTFGGGTKVEIKR

SEQ ID NO: 9 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 10 - DNA, 994 bp, Homo sapiens
aagactcaaacttagaaacttgaattagatgtggtattcaaatccttagctgccgcgaagacacagacagcccccgtaagaacccacgaagcaggcgaagttcattgttctcaacattctagctgctcttgctgcatttgctctggaattcttgtagagatattacttgtccttccaggctgttctttctgtagctcccttgttttctttttgtgatcatgttgcagatggctgggcagtgctcccaaaatgaatattttgacagtttgttgcatgcttgcataccttgtcaacttcgatgttcttctaatactcctcctctaacatgtcagcgttattgtaatgcaagtgtgaccaattcagtgaaaggaacgaatgcgattctctggacctgtttgggactgagcttaataatttctttggcagttttcgtgctaatgtttttgctaaggaagataaactctgaaccattaaaggacgagtttaaaaacacaggatcaggtctcctgggcatggctaacattgacctggaaaagagcaggactggtgatgaaattattcttccgagaggcctcgagtacacggtggaagaatgcacctgtgaagactgcatcaagagcaaaccgaaggtcgactctgaccattgctttccactcccagctatggaggaaggcgcaaccattcttgtcaccacgaaaacgaatgactattgcaagagcctgccagctgctttgagtgctacggagatagagaaatcaatttctgctaggtaattaaccatttcgactcgagcagtgccactttaaaaatcttttgtcagaatagatgatgtgtcagatctctttaggatgactgtatttttcagttgccgatacagctttttgtcctctaactgtggaaactctttatgttagatatatttctctaggttactgttgggagcttaatggtagaaacttccttggtttcatgattaaactcttttttttcctga

SEQ ID NO: 11 - DNA, 363 bp, Artificial
  Heavy Chain Variable Domain
caagtgcagctggtgcagtccggagcggaagtgaagaaacctggggcgtccgtgaagctcagctgcaaggcctccggctacactttcaccgattactacatccactgggtcagacaggcaccgggacagggactggagtggattggttacatcaaccccaactccgggtacaccaattacgcccagaagttccagggtcgggctacgatgaccgccgacaagtcgatcaacactgcctacgtggaactgtcaaggctgcggtccgatgacaccgccgtgtacttctgtacccgctatatgtgggagcgcgtgactggatttttcgacttctggggccaaggcaccatggtcaccgtgtcgagc

SEQ ID NO: 12 - DNA, 324 bp, Artificial
  Light Chain Variable Domain
gacattcagatgacccagtccccctcgtccgtgtccgcttccgtgggagatcgcgtgaccatcacttgtcttgcgtccgaggatatctcagacgacctggcctggtaccagcagaagcctggaaaggccccgaaggtcctggtgtacactaccagcagcctccagtcgggcgtgccttcacggttctccggttcggggtctggcaccgacttcaccctgactattagctccctgcaacccgaggacttcgccacctacttttgccagcaaacctacaagttcccgccaacgttcggagggggcaccaaggtcgaaatcaaacgt

SEQ ID NO: 13 - protein, 451 aa, Artificial
  Heavy Chain
QVQLVQSGAEVKKPGASVKLSCKASGYTFTDYYIHWVRQAPGQGLEWIGYINPNSGYTNYAQKFQGRATMTADKSINTAYVELSRLRSDDTAVYFCTRYMWERVTGFFDFWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - DNA, 1353 bp, Artificial
  Heavy Chain
caagtgcagctggtgcagtccggagcggaagtgaagaaacctggggcgtccgtgaagctcagctgcaaggcctccggctacactttcaccgattactacatccactgggtcagacaggcaccgggacagggactggagtggattggttacatcaaccccaactccgggtacaccaattacgcccagaagttccagggtcgggctacgatgaccgccgacaagtcgatcaacactgcctacgtggaactgtcaaggctgcggtccgatgacaccgccgtgtacttctgtacccgctatatgtgggagcgcgtgactggatttttcgacttctggggccaaggcaccatggtcaccgtgtcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggccgtcctacagtcctcaggactctactccctcagcagcgtggtgaccgtgccctccagcagcttgggcacccagacctacatctgcaacgtgaatcacaagcccagcaacaccaaggtggacaagaaggttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 15 - protein, 214 aa, Artificial
  Light Chain
DIQMTQSPSSVSASVGDRVTITCLASEDISDDLAWYQQKPGKAPKVLVYTTSSLQSGVPSRFSGSGSGTDFTLTISSLQPEDFATYFCQQTYKFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - DNA, 642 bp, Artificial
  Light Chain
gacattcagatgacccagtccccctcgtccgtgtccgcttccgtgggagatcgcgtgaccatcacttgtcttgcgtccgaggatatctcagacgacctggcctggtaccagcagaagcctggaaaggccccgaaggtcctggtgtacactaccagcagcctccagtcgggcgtgccttcacggttctccggttcggggtctggcaccgacttcaccctgactattagctccctgcaacccgaggacttcgccacctacttttgccagcaaacctacaagttcccgccaacgttcggagggggcaccaaggtcgaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

(19) United States
(12) Patent Application Publication
(10) Pub. No.: US 2023/0118517 A1
(43) Pub. Date: Apr. 20, 2023

(54) METHODS OF TREATING MULTIPLE MYELOMA

(71) Applicant: Seagen Inc., Bothell, WA (US)

(72) Inventors: Mary Campbell, Bothell, WA (US); Heather Van Epps, Bothell, WA (US); Haley Neff-LaFord, Bothell, WA (US); Celine Fabienne Jacquemont, Bothell, WA (US); David Taft, Bothell, WA (US); Hong Li, Bothell, WA (US)

(21) Appl. No.: 17/913,542

(22) PCT Filed: Mar. 25, 2021
(86) PCT No.: PCT/US2021/024127

§ 371 Date: Sep. 22, 2022

Related U.S. Application Data
(60) Provisional application No. 63/000,229, filed on Mar. 26, 2020.

(51) Int. Cl. C07K 16/28; A61P 35/00

(52) U.S. Cl. CPC C07K 16/2875; C07K 16/2896; A61P 35/00; C07K 2317/565; C07K 2317/24; A61K 2039/545

(57) ABSTRACT

Provided herein are methods of treating multiple myeloma (MM) using specific doses of an anti-B-cell migration antigen (BCMA) antibody, various dosing regimens, and optionally combination therapy with dexamethasone, an immunomodulatory agent (e.g., pomalinamide), an anti-CD38 antibody or antigen binding fragment thereof (e.g., daratumumab), and a gamma secretase inhibitor (GSI), and/or various combinations thereof.

### CLAIM OF PRIORITY

This application claims the benefit of U.S. Provisional Application Ser. No. 63/000,229, filed on Mar. 26, 2020. The entire contents of the foregoing are incorporated herein by reference.

### BACKGROUND

Multiple Myeloma (MM) is a neoplastic disorder of clonally proliferating plasma cells in the bone marrow, peripheral blood, or other extramedullary sites. Malignant plasma cells exert a direct pathologic effect on the marrow microenvironment and adjacent skeletal bone, leading to anemia, osteolytic bone lesions, and hypercalcemia. In most cases, malignant plasma cells also produce an abnormal monoclonal immunoglobulin known as the M protein, but in a minority of subjects, the myeloma cells produce only monoclonal free light chains (FLC). Abnormal levels of either M protein or FLC can contribute to the clinical spectrum of disease that includes renal failure and an increased susceptibility to infections (Kumar et al., Nat. Rev. Dis. Primers 3:17046, 2017; Palumbo et al., N. Engl. J. Med. 364(11):1046-1060, 2011; Rollig et al., Lancet 385(9983):2197-2208, 2015).

Standard treatments for MM include combination chemotherapy regimens containing proteasome inhibitors (PIs) such as bortezomib and carfilzomib, and ixazomib, and/or immunomodulatory drugs (IMiDs), such as lenalidomide and pomalidomide. Alkylating agents such as melphalan and cyclophosphamide are also active in MM. Patients who are free from significant comorbidities and considered eligible, are often treated with myeloablative chemotherapy and/or radiation, followed by autologous stem cell transplant (ASCT) (Rollig et al., Lancet. 385(9983):2197-208, 2015; and Rajkumar et al., Mayo Clin Proc. 91(1):101-19, 2016). More recently, daratumumab, a monoclonal antibody targeting the CD38 antigen, has been approved for the treatment of RRMM as monotherapy in fourth line therapy.

To date, multiple myeloma remains an incurable disease managed with sequential lines of treatment that typically yield shorter durations of disease control with each subsequent relapse (Kumar et al., Mayo Clin. Proc. 79(7):867-874, 2004).

### SUMMARY

In one aspect, this application is based in part on the discovery of methods that result in, e.g., one or more of: a therapeutically desired steady-state concentration of an anti-BCMA antibody in the serum of a subject, a therapeutically desired reduction in the steady-state levels of free light chain in the serum of a subject, and a therapeutically desired saturation of BCMA in a subject.

In another aspect, this application is based upon evidence demonstrating the efficacy of combining certain BCMA therapeutic agents such as BCMA antibodies, including non-fucosylated antibodies, with various other therapeutics to treat cancers such as MM. Therapeutics found to successfully combine with such BCMA agents (e.g., non-fucosylated antibodies) include dexamethasone, an immunomodulatory agent (an IMiD) (e.g., pomalinamide), an CD38 antibody (e.g., daratumumab), and/or a gamma secretase inhibitor (GSI), including all the various combinations of these therapeutic agents.

In yet another aspect, the application is based in part on the identification of various BCMA antibody dosing regimens, including a standard and an intensive dosing regimen (defined more fully below) that have been shown to be therapeutically efficacious as monotherapy as well as in combination therapy, including combinations with dexamethasone, an IMiD (e.g., pomalinamide), an CD38 antibody (e.g., daratumumab), and/or a GSI. These results were unexpected given that a relatively high level of a BCMA antibody as described herein could be administered while still maintaining a manageable safety profile, including even when the BCMA antibody was administered as part of a combination therapy.

Accordingly, provided herein are methods of treating a subject having multiple myeloma (MM), the method comprising administering to the subject one or more doses of an antibody, or antigen binding fragment thereof, that specifically binds to a B cell maturation antigen (BCMA), and wherein the one or more doses are independently administered to the subject at about 100 mg of the antibody or antigen-binding fragment to about 2,000 mg of the antibody or antigen-binding fragment.

In some embodiments of any of the methods described herein, the antibody or antigen-binding fragment thereof is a non-fucosylated antibody or antigen-binding fragment.

In some embodiments, a composition comprising the antibody or antigen-binding fragment thereof is administered to the subject. In some embodiments, about or at least 95%, 97%, 98% or 99% of the antibody or antigen-binding fragment thereof in the composition are afucosylated.

In some embodiments of any of the methods described herein, the antibody or antigen binding-fragment thereof, comprises: a heavy chain variable region comprising a CDR1 comprising SEQ ID NO: 1, a CDR2 comprising SEQ ID NO: 2, and a CDR3 comprising SEQ ID NO: 3, and a light chain variable domain comprising a CDR1 comprising SEQ ID NO: 5, a CDR2 comprising SEQ ID NO: 6, and a CDR3 comprising SEQ ID NO: 7.

In some embodiments of any of the methods described herein, the antibody or the antigen-binding fragment thereof comprises a heavy chain variable domain comprising an amino acid sequence that is at least 80% identical to SEQ ID NO: 4 and a light chain variable domain comprising an amino acid sequence that is at least 80% identical to SEQ ID NO: 8.

In some embodiments of any of the methods described herein, the antibody or the antigen-binding fragment thereof comprises a heavy chain variable domain comprising an amino acid sequence that is at least 90% identical to SEQ ID NO: 4 and a light chain variable domain comprising an amino acid sequence that is at least 90% identical to SEQ ID NO: 8.

In some embodiments of any of the methods described herein, the antibody or the antigen-binding fragment thereof comprises a heavy chain variable domain comprising an amino acid sequence of SEQ ID NO: 4 and a light chain variable domain comprising an amino acid sequence of SEQ ID NO: 8.

In some embodiments of any of the methods described herein, the antibody or the antigen-binding fragment is humanized.

In some embodiments of any of the methods described herein, the antibody is an IgG1 antibody. In some embodiments, the antibody or antigen-binding fragment is not a bispecific antibody, a bispecific T cell engager (BiTE), a chimeric antigen receptor (CAR), or an antibody drug conjugate (ADC), or a portion thereof.

In some embodiments of any of the methods described herein, the one or more doses are independently administered to the subject at about 800 mg of the antibody or antigen-binding fragment to about 2,000 mg of the antibody or antigen-binding fragment.

In some embodiments of any of the methods described herein, the one or more doses are independently administered to the subject at about 1,200 mg of the antibody or antigen-binding fragment to about 2,000 mg of the antibody or antigen-binding fragment.

In some embodiments of any of the methods described herein, the one or more doses are independently administered to the subject at about 1,400 mg of the antibody or antigen-binding fragment to about 1,800 mg of the antibody or antigen-binding fragment.

In some embodiments of any of the methods described herein, the one or more doses are independently administered to the subject at about 400 mg of the antibody or antigen-binding fragment.

In some embodiments of any of the methods described herein, the one or more doses are independently administered to the subject at about 800 mg of the antibody or antigen-binding fragment.

In some embodiments of any of the methods described herein, the one or more doses are administered to the subject at about 1,600 mg of the antibody or antigen-binding fragment.

In some embodiments of any of the methods described herein, two or more doses of the antibody or antigen-binding fragment are administered to the subject.

In some embodiments of any of the methods described herein, the two or more doses are administered to the subject at a frequency of between once a week and about once every four weeks.

In some embodiments of any of the methods described herein, the two or more doses are administered to the subject at a frequency of about once a week.

In some embodiments of any of the methods described herein, the two or more doses are administered to the subject at a frequency of about once every two weeks.

In some embodiments of any of the methods described herein, the two or more doses are administered to the subject at a frequency of about once every three weeks.

In some embodiments of any of the methods described herein, the two or more doses are administered to the subject at a frequency of about once every four weeks.

In some embodiments of any of the methods described herein, each dose comprises about 800 mg of the antibody or antigen-binding fragment and is administered to the subject every 2 weeks.

In some embodiments of any of the methods described herein, each dose comprises about 1600 mg of the antibody or antigen-binding fragment and is administered to the subject every 2 weeks.

In some embodiments of any of the methods described herein, individual doses of the antibody or antigen-binding fragment are administered to the subject on day 1 and day 15 of a 28-day cycle.

In some embodiments of any of the methods described herein, the doses of the antibody or antigen-binding fragment are administered to the subject for multiple 28-day cycles.

In some embodiments of any of the methods described herein, the one or more doses comprise one or more induction doses that are administered to the subject during an induction phase and one or more maintenance doses that are administered to the subject during a maintenance phase after the one or more induction doses have been administered.

In some embodiments of any of the methods described herein, one of the induction doses is administered to the subject about once a week for about 1-10 weeks.

In some embodiments of any of the methods described herein, one of the induction doses is administered to the subject once a week for 8 weeks.

In some embodiments of any of the methods described herein, one of the induction doses is administered 4 times within a 28-day cycle.

In some embodiments of any of the methods described herein, one of the induction doses is administered 8 times within two 28-day cycles.

In some embodiments of any of the methods described herein, one of the induction doses is independently administered on day 1, day 8, day 15 and day 22 for each of two 28-day cycles.

In some embodiments of any of the methods described herein, each induction dose comprises about 100, 200, 400, 800, or 1600 mg of the antibody or antigen-binding fragment.

In some embodiments of any of the methods described herein, each induction dose comprises about 800 mg of the antibody or antigen-binding fragment.

In some embodiments of any of the methods described herein, each induction dose comprises about 1600 mg of the antibody or antigen-binding fragment.

In some embodiments of any of the methods described herein, the one or more maintenance doses are administered once every 1-4 weeks after completion of the induction phase.

In some embodiments of any of the methods described herein, one of the maintenance doses is administered once every two weeks.

In some embodiments of any of the methods described herein, one of the maintenance doses is administered on day 1 and day 15 of a 28-day cycle.

In some embodiments of any of the methods described herein, each maintenance dose comprises about 100, 200, 400, 800, or 1600 mg of the antibody or antigen-binding fragment.

In some embodiments of any of the methods described herein, each maintenance dose comprises about 800 mg of the antibody or antigen-binding fragment.

In some embodiments of any of the methods described herein, each maintenance dose comprises about 1600 mg of the antibody or antigen-binding fragment.

In some embodiments of any of the methods described herein, the antibody or antigen-binding fragment is dosed q1wk during the induction phase for a total of 8 induction phase doses and dosed q2wk during the maintenance phase.

In some embodiments of any of the methods described herein, each induction dose comprises about 100, 200, 400, or 1600 mg of the antibody or antigen-binding fragment; each maintenance dose comprises about 100, 200, 400, or 1600 mg of the antibody or antigen-binding fragment; one of the induction doses is administered on each of day 1, day 8, day 15 and day 22 for each of two 28-day cycles for a total of 8 induction doses during the induction phase; and one of the maintenance doses is administered on each of days 1 and day 15 of each of one or more subsequent cycles.

In some embodiments of any of the methods described herein, each induction dose and each maintenance dose comprises about 800 or 1600 mg of the antibody or antigen-binding fragment.

In some embodiments of any of the methods described herein, each induction dose and each maintenance dose comprises about 1600 mg of the antibody or antigen-binding fragment.

In some embodiments of any of the methods described herein, the method further comprises administering one or more doses of dexamethasone to the subject.

In some embodiments of any of the methods described herein, the one or more doses of dexamethasone are independently administered to the subject at a frequency of once a week.

In some embodiments of any of the methods described herein, one of the doses of the antibody or antigen-binding fragment are administered at a frequency of about once every 1-4 weeks and the doses of dexamethasone are administered at a frequency of about once every 1-4 weeks.

In some embodiments of any of the methods described herein, one of the doses of the antibody or antigen binding fragment are administered once every two weeks and one of the doses of dexamethasone is administered once every two weeks.

In some embodiments of any of the methods described herein, one of the doses of the antibody or antigen binding fragment is administered once every two weeks and one of the doses of dexamethasone is administered once every week.

In some embodiments of any of the methods described herein, one of the doses of the antibody or antigen binding fragment is administered on each of day 1 and day 15 of a 28-day cycle and one of the doses of dexamethasone is administered on each of day 1, day 8, day 15 and day 22 of the same 28-day cycle.

In some embodiments of any of the methods described herein, one of the doses of the antibody or antigen-binding fragment is administered once a week during an induction phase with subsequent doses following the induction phase being administered once every two weeks during a maintenance phase; and one of the doses of dexamethasone is administered once every week.

In some embodiments of any of the methods described herein, one of the doses of the antibody or antigen-binding fragment is administered once a week for 8 weeks during the induction phase and subsequent doses are administered once every two weeks during the maintenance phase; and one of the doses of dexamethasone is administered once every week.

In some embodiments of any of the methods described herein, one of the doses of the antibody or antigen-binding fragment is administered on each of day 1, day 8, day 15, and day 22 of each of two 28-day cycles and then on each of day 1 and day 15 of subsequent 28-day cycles; and one of the doses of dexamethasone is administered on each of day 1, day 8, day 15, and day 22 of each of the 28-day cycles.

In some embodiments of any of the methods described herein, when the antibody or antigen-binding fragment are administered on the same day then dexamethasone is administered about 1-3 hours before the antibody or antigen binding fragment is administered.

In some embodiments of any of the methods described herein, each dose of the antibody or antigen-binding fragment is administered as an about 800 mg dose.

In some embodiments of any of the methods described herein, each dose of the antibody or antigen-binding fragment is administered as an about 1600 mg dose.

In some embodiments of any of the methods described herein, each dose of dexamethasone is administered as an about 20 to about 60 mg dose.

In some embodiments of any of the methods described herein, each dose of dexamethasone is administered as an about 40 mg dose or an about 20 mg dose.

In some embodiments of any of the methods described herein, each dose of the antibody or antigen-binding fragment is administered as an about 1600 mg dose, and wherein each dose of dexamethasone is administered as an about 40 mg dose.

In some embodiments, the method further comprises administering one or more doses of an anti-CD38 antibody, or antigen-binding fragment thereof to the subject. In some embodiments, the anti-CD38 antibody is daratumumab.

In some embodiments, the one or more doses of the anti-CD38 antibody or antigen binding fragment thereof are independently administered to the subject at about 5 mg/kg (milligram per kilogram of body weight) to about 30 mg/kg. In some embodiments, the one or more doses of the anti-CD38 antibody or antigen-binding fragment thereof are independently administered to the subject at about 10 mg/kg to about 20 mg/kg. In some embodiments, the one or more doses of the anti-CD38 antibody or antigen-binding fragment thereof are independently administered to the subject at about 16 mg/kg.

In some embodiments, two or more doses of the anti-CD38 antibody or antigen-binding fragment are administered to the subject. In some embodiments, the two or more doses of the anti-CD38 antibody or antigen binding fragment thereof are administered to the subject at a frequency of about once a week to about once every four weeks. In some embodiments, the two or more doses of the anti-CD38 antibody or antigen-binding fragment thereof are administered to the subject at a frequency of about once a week. In some embodiments, the two or more doses of the anti-CD38 antibody or antigen-binding fragment thereof are administered to the subject at a frequency of about once every two weeks or once every three weeks. In some embodiments, the two or more doses of the anti-CD38 antibody or antigen-binding fragment thereof are administered to the subject at a frequency of about once every four weeks. In some embodiments, the anti-CD38 antibody or antigen-binding fragment thereof are administered to the subject on day 1, day 8, day 15, and day 22 for 28-day cycles.

In some embodiments, two or more doses of the anti-CD38 antibody or antigen-binding fragment thereof are administered to the subject at a frequency of about once every week during a first phase; two or more doses of the anti-CD38 antibody or antigen binding-fragment thereof are administered to the subject at a frequency of about once every two weeks to about once every three weeks during a second phase; and two or more doses of the anti-CD38 antibody or antigen binding fragment thereof are administered to the subject at a frequency of about once every four weeks during a third phase.

In some embodiments, the first phase is about 6 weeks to about 10 weeks. In some embodiments, the first phase is about 8 weeks or about 9 weeks. In some embodiments, the second phase is about 10 weeks to about 20 weeks.

In some embodiments, 8 doses of the anti-CD38 antibody or antigen-binding fragment thereof are administered to the subject at a frequency of about once every two weeks during the second phase. In some embodiments, 5 doses of the anti-CD38 antibody or antigen-binding fragment thereof are administered to the subject at a frequency of about once every three weeks during the second phase.

In some embodiments, multiple doses of the anti-CD38 antibody or antigen-binding fragment thereof are administered to the subject at a frequency of about once every four weeks during the third phase until disease progression.

In some embodiments, the method further comprises administering one or more doses of an immunomodulatory drug. In some embodiments, the immunomodulatory drug is an immunomodulatory imide drug (IMiD). In some embodiments, the immunomodulatory drug is lenalidomide or pomalidomide.

In some embodiments, the immunomodulatory drug is pomalidomide.

In some embodiments, the one or more doses of the immunomodulatory drug are independently administered to the subject at a frequency of about once per day to about once per week. In some embodiments, the one or more doses of the immunomodulatory drug are independently administered to the subject once per day. In some embodiments, the one or more doses of the immunomodulatory drug are independently administered to the subject once per day on Days 1-21 of repeated 28-day cycles.

In some embodiments, each dose of the immunomodulatory drug is about 1 mg to about 10 mg. In some embodiments, each dose of the immunomodulatory drug is about 2 mg to about 4 mg. In some embodiments, each dose of the immunomodulatory drug is about 4 mg.

In some embodiments, when a dose of the immunomodulatory drug and a dose of the antibody or antigen-binding fragment that specifically binds to BCMA are administered on the same day, the dose of the immunomodulatory drug is administered about 1 to about 3 hours before the dose of the antibody or antigen-binding fragment that specifically binds to BCMA.

In some embodiments, the antibody or antigen-binding fragment that specifically binds to BCMA is administered to the subject on day 1 and day 15 of a 28-day cycle, dexamethasone is administered to the subject on day 1, 8, 15, and 22 of the 28-day cycle, and pomalidomide is administered to the subject on days 1-21 of the 28-day cycle.

In some embodiments, one of the induction doses of the antibody or antigen-binding fragment that specifically binds to BCMA is administered on each of day 1, day 8, day 15 and day 22 for two 28-day cycles for a total of 8 induction doses during the induction phase; and one of the maintenance doses of the antibody or antigen-binding fragment that specifically binds to BCMA is administered on each of days 1 and day 15 of each of one or more subsequent 28-day cycles in the maintenance phase; In some embodiments, dexamethasone is administered to the subject on each of day 1, 8, 15, and 22 of each 28-day cycle in the induction phase and the maintenance phase. In some embodiments, pomalidomide is administered to the subject on days 1-21 of each 28-day cycle in the induction phase and the maintenance phase.

In some embodiments, when a dose of dexamethasone and a dose of the antibody or antigen-binding fragment are administered on the same day or a dose of pomalidomide and a dose of the antibody or antigen-binding fragment are administered on the same day, the dose of dexamethasone or the dose of pomalidomide is administered about 1 to about 3 hours before the dose of the antibody or antigen-binding fragment.

In some embodiments, the method further comprises administering one or more doses of a gamma-secretase inhibitor to the subject. In some embodiments, the gamma-secretase inhibitor is Semagacestat (LY450139), R04929097, MK-0752, Avagacestat (BMS-708163), BMS-986115, Nirogacestat (PF-03084014), Crenigacestat (LY3039478), BMS-906024, DAPT (GSI-IX), Dibenzazepine (YO-01027), LY411575, L-685458, NGP 555, MDL-28170, or Itanapraced (CHF 5074). Some of these gamma-secretase inhibitors are described in e.g. WO2019094626A1, U.S. Pat. Nos. 9,914,774, 6,756,511, 6,890,956, 6,984,626, 7,049,296, 7,101,895, 7,138,400, 7,144,910, 7,183,303, 8,377,886, WO 2002/40451A2, U.S. Pat. No. 7,468,365B2, US20160354382, US2020179511A1, US2019367628A1, US2020085839A1, U.S. Ser. No. 10/590,087, US2020171020A1, US662510A, U.S. Pat. No. 4,434,171A, US2019367628A1, US2019367628A1, U.S. Pat. No. 7,244,739, US2020087623A1, U.S. Ser. No. 10/307,388B2, and PubChem of the NCBI website; each of which is incorporated herein by the reference in its entirety.

In some embodiments of any of the methods described herein, each dose of the antibody or antigen-binding fragment is administered by systemic administration.

In some embodiments of any of the methods described herein, the systemic administration is by intravenous administration.

In some embodiments of any of the methods described herein, at least the initial dose of the antibody or antigen-binding fragment is administered to the subject using step-wise infusion.

In some embodiments of any of the methods described herein, the step-wise infusion is performed using an infusion rate of about 50 mg/hour to about 400 mg/hour.

In some embodiments of any of the methods described herein, the step-wise infusion, the infusion rate is increased every 30 minutes.

In some embodiments of any of the methods described herein, the step-wise infusion, the infusion rate is increased no more than two-fold every 30 minute.

In some embodiments of any of the methods described herein, the subject is a human subject.

In some embodiments of any of the methods described herein, the subject has previously been diagnosed as having multiple myeloma.

In some embodiments of any of the methods described herein, the subject has been diagnosed as having relapsed or refractory multiple myeloma.

In some embodiments of any of the methods described herein, the subject was previously administered one or more therapeutic agents or treatments for multiple myeloma.

In some embodiments of any of the methods described herein, the previously administered one or more therapeutic agents or treatments for multiple myeloma were unsuccessful.

In some embodiments of any of the methods described herein, the subject has previously been administered at least one of a proteasome inhibitor, an immunomodulatory agent, and an anti-CD38 antibody, or cannot tolerate the foregoing.

In some embodiments of any of the methods described herein, the subject has previously been administered therapeutic agents include all three of a proteasome inhibitor, an immunomodulatory agent, and an anti-CD38 antibody, or cannot tolerate the foregoing.

In some embodiments of any of the methods described herein, the subject has previously been administered at least 3 prior lines of anti-myeloma therapy and is refractory to at least one therapeutic agent in each of the following classes: a proteasome inhibitor, an immunomodulatory agent, and an anti-CD38 antibody.

In some embodiments of any of the methods described herein, the subject satisfies 1, 2 or all 3 of the following criteria prior to initiating treatment: serum monoclonal paraprotein (M-protein) level of ≥about 0.5 g/dL, urine M-protein level ≥about 200 mg/24 hr, and serum immunoglobulin free light chain ≥about 10 mg/dL and abnormal serum immunoglobulin kappa lambda free light chain ratio.

In some embodiments of any of the methods described herein, the method results in a steady-state concentration of the antibody, or antigen-binding fragment thereof, in the serum of the subject of about 1 μg/mL to about 200 μg/mL.

In some embodiments of any of the methods described herein, the method results in a steady-state concentration of free light chain (FLC) in the serum of the subject of less than about 50 mg/dL.

In some embodiments, the subject has received at least two prior lines of anti-myeloma therapy (e.g., including at least 2 consecutive cycles of lenalidomide and a proteosome inhibitor (e.g., given separately or in combination)), and has documented IMWG (International Myeloma Working Group) disease progression on or within 60 days of completion of the two prior lines of anti-myeloma therapy.

In some embodiments of any of the methods described herein, one or more therapeutic effects in the subject is improved after administration of the antibody-drug conjugate relative to a baseline.

In some embodiments of any of the methods described herein, the one or more therapeutic effects is selected from the group consisting of: objective response rate, complete response rate, duration of response, duration of complete response, time to response, progression free survival, and overall survival.

In some embodiments of any of the methods described herein, the objective response rate is at least about 20%, at least about 25%, at least about 30%, at least about 35%, at least about 40%, at least about 45%, at least about 50%, at least about 60%, at least about 70%, or at least about 80%.

In some embodiments of any of the methods described herein, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years.

In some embodiments of any of the methods described herein, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years.

In some embodiments of any of the methods described herein, the duration of response or the duration of complete response to the treatment is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years.

Also provided are kits comprising: (a) one or more doses of a pharmaceutical composition comprising (i) an antibody, or antigen binding fragment thereof, that specifically binds to a B cell maturation antigen (BCMA), and (ii) a pharmaceutically acceptable carrier, wherein the antibody or antigen binding fragment thereof, comprises: a heavy chain variable region comprising a CDR1 comprising SEQ ID NO: 1, a CDR2 comprising SEQ ID NO: 2, and a CDR3 comprising SEQ ID NO: 3, and a light chain variable domain comprising a CDR1 comprising SEQ ID NO: 5, a CDR2 comprising SEQ ID NO: 6, and a CDR3 comprising SEQ ID NO: 7, and (b) instructions for performing any of the methods described herein.

Also provided herein are methods of treating a subject having multiple myeloma, the method including administering to the subject one or more doses of a pharmaceutical composition comprising (i) an antibody, or antigen-binding fragment thereof, that specifically binds to a B cell maturation antigen (BCMA), and (ii) a pharmaceutically acceptable carrier.

In some embodiments of any of the methods described herein, the multiple myeloma is relapsed or refractory multiple myeloma (RRMM). In some embodiments, the antibody, or antigen-binding fragment thereof comprises: a heavy chain variable region comprising a CDR1 comprising SEQ ID NO: 1, a CDR2 comprising SEQ ID NO: 2, and a CDR3 comprising SEQ ID NO: 3, and a light chain variable domain comprising a CDR1 comprising SEQ ID NO: 5, a CDR2 comprising SEQ ID NO: 6, and a CDR3 comprising SEQ ID NO: 7.

In some embodiments, the antibody is an IgG1 antibody. In some embodiments, one or more doses of about 1600 mg of the antibody, or antigen-binding fragment thereof, is independently administered to the subject at a frequency of every two weeks. In some embodiments, one or more doses of about 800 mg of the antibody, or antigen-binding fragment thereof, is independently administered to the subject at a frequency of every week. In some embodiments, about 1-2 induction doses of about 1600 mg of the antibody, or antigen-binding fragment thereof, is independently administered to the subject at a frequency of every week, followed by one or more maintenance doses of about 1600 mg of the antibody, or antigen-binding fragment thereof, independently administered to the subject at a frequency of every two weeks. In some embodiments, about 1-2 induction doses of about 800 mg of the antibody, or antigen-binding fragment thereof, is independently administered to the subject at a frequency of every week, followed by one or more maintenance doses of about 1600 mg of the antibody, or antigen-binding fragment thereof, independently administered to the subject at a frequency of every two weeks. In some embodiments, a dose of about 40 mg of dexamethasone is administered to the subject about 1 to about 3 hours prior to the administration of each dose of the antibody, or antigen-binding fragment thereof.

In some embodiments, the subject was previously administered one or more therapeutic agents or treatments for multiple myeloma. The one or more previously administered therapeutic agents or treatments for multiple myeloma include, but are not limited to, a proteasome inhibitor (PI), an immunomodulatory drug (IMiD), and an anti-CD38 antibody. In some embodiments, the previously administered therapeutic agents or treatments for multiple myeloma were not effective in treating the multiple myeloma. In some embodiments, the subject has one or more of: a serum monoclonal paraprotein (M-protein) level of ≥about 0.5 g/dL, a urine M-protein level of ≥about 200 mg/24 hours, a serum immunoglobulin free light chain ≥about 10 mg/dL, and an abnormal serum immunoglobulin kappa to lambda free light chain ratio.

Also provided herein are kits that comprise or consist of (a) one or more doses of a pharmaceutical composition comprising (i) an antibody, or antigen-binding fragment thereof, that specifically binds to a B cell maturation antigen (BCMA), and (ii) a pharmaceutically acceptable carrier, wherein the antibody or antigen-binding fragment thereof, comprises: a heavy chain variable region comprising a CDR1 comprising SEQ ID NO: 1, a CDR2 comprising SEQ ID NO: 2, and a CDR3 comprising SEQ ID NO: 3, and a light chain variable domain comprising a CDR1 comprising SEQ ID NO: 5, a CDR2 comprising SEQ ID NO: 6, and a CDR3 comprising SEQ ID NO: 7; and optionally (b) instructions for performing a treatment method as described herein.

In some embodiments, the kit further comprises one or more doses of dexamethasone, one or more doses of an immunomodulatory imide drug, one or more doses of a gamma-secretase inhibitor, and/or one or more doses of an anti-CD38 antibody or antigen binding fragment thereof.

In some embodiments, the kit further comprises one or more doses of dexamethasone.

In some embodiments, the kit further comprises one or more doses of an immunomodulatory imide drug.

In some embodiments, the kit further comprises one or more doses of dexamethasone and one or more doses of an immunomodulatory imide drug.

In some embodiments, the kit further comprises one or more doses of a gamma-secretase inhibitor.

In some embodiments, the kit further comprises one or more doses of an anti-CD38 antibody.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a graph showing the mean serum concentration of the SEA-BCMA antibody over time in subjects administered different doses of SEA-BCMA.

FIG. 1B shows the results of the pharmacokinetic analysis of SEA-BCMA depicted in FIG. 1A.

FIG. 2A is a schematic illustration showing the detection of free SEA-BCMA using an in vitro cell binding capacity assay.

FIG. 2B shows the standard curves generated using the assay depicted in FIG. 2A.

FIG. 3A is a graph showing the percentage of membrane-bound BCMA in an in vitro membrane BCMA assay performed using serum from subjects administered 100 mg, 200 mg, or 400 mg doses of SEA-BCMA, with each line corresponding to a different subject.

FIG. 3B is a graph showing the percentage of membrane-bound BCMA in an in vitro membrane BCMA assay performed using serum from subjects administered 800 mg doses of SEA-BCMA, with each line corresponding to a different subject.

FIG. 3C is a graph showing the percentage of membrane-bound BCMA in an in vitro membrane BCMA assay performed using serum from subjects administered 1600 mg doses of SEA-BCMA.

FIG. 4 is a bar graph showing the binding of membrane BCMA on the surface of malignant plasma cells.

FIG. 5 shows previous treatments administered to one patient and results after treatment with SEA-BCMA.

FIG. 6A is a graph showing serum free light chain (sFLC) levels in a patient (Patient A) after administration of a 1600 mg dose of SEA-BCMA.

FIG. 6B shows higher levels of membrane-bound BCMA at baseline in Patient A, who saw a marked decrease in sFLC as shown in FIG. 6A, compared to several other patients enrolled in the study.

FIG. 7 shows previous treatments administered to one patient and results after treatment with SEA-BCMA.

FIG. 8A. NCI-H929 cells displayed increased BCMA expression upon DAPT treatment. Light gray: isotype control; medium gray: untreated cells; dark gray: DAPT treated cells.

FIG. 8B. Molp-8 cells displayed increased BCMA expression upon DAPT treatment. Light gray: isotype control; medium gray: untreated cells; dark gray: DAPT treated cells.

FIG. 8C. Fold over background of NFAT signaling due to FcγRIII engagement. DAPT (GSI) treated NCI-H929 cells compared to untreated cells (N=3).

FIG. 8D. Fold over background of NFAT signaling due to FcγRIII engagement. DAPT (GSI) treated Molp-8 cells compared to untreated cells (N=3).

FIG. 9A. Molp-8 cells displayed increased BCMA expression upon Nirogacestat treatment. Dark gray: isotype control; medium gray: untreated cells; light gray: Nirogacestat treated cells.

FIG. 9B. The maximum percentage of target cell lysis of Nirogacestat treated cells compared to untreated cells (N=3). hIgG1k is a non-binding antibody control.

FIG. 10. p65 activation of NCI-H929 cells bound with and without SEA-BCMA, treated with and without APRIL, in the presence or absence of Nirogacestat.

FIG. 11. Mean luminescence plotted for 5 animals after initiation of dosing on day 18 after luminescent Molp-8 cells were implanted. Luminescence was evaluated over a 100 day period.

FIG. 12A. SEA-BCMA combined with Pomalidomide induced greater MM1R target cell killing in vitro benchmarked against elotuzumab and WT-BCMA. (N=2)

FIG. 12B. SEA-BCMA combined with Pomalidomide induced greater MM1R target cell killing in vitro benchmarked against daratumumab. (N=2)

### DETAILED DESCRIPTION

Provided herein are methods of treating a subject having multiple myeloma (MM) that comprise administering to the subject one or more doses of a an antibody that binds to B cell maturation antigen (BCMA), or antigen-binding fragment thereof.

In some embodiments, the antibody is an IgG1 antibody. In some embodiments, the antibody is an afucosylated antibody. In some embodiments, the antibody or antigen-binding fragment thereof, comprises: a heavy chain variable region comprising a CDR1 comprising SEQ ID NO: 1, a CDR2 comprising SEQ ID NO: 2, and a CDR3 comprising SEQ ID NO: 3, and a light chain variable domain comprising a CDR1 comprising SEQ ID NO: 5, a CDR2 comprising SEQ ID NO: 6, and a CDR3 comprising SEQ ID NO: 7. In some embodiments, one or more doses of 1600 mg of the antibody, or antigen-binding fragment thereof, is independently administered to the subject at a frequency of every two weeks. In some embodiments, one or more doses of 800 mg of the antibody, or antigen-binding fragment thereof, is independently administered to the subject at a frequency of every week. In some embodiments, about 1-2 induction doses of 1600 mg of the antibody, or antigen-binding fragment thereof, is independently administered to the subject at a frequency of every week, followed by one or more maintenance doses of 1600 mg of the antibody, or antigen-binding fragment thereof, independently administered to the subject at a frequency of every two weeks. In some embodiments, about 1-2 induction doses of 800 mg of the antibody, or antigen-binding fragment thereof, is independently administered to the subject at a frequency of every week, followed by one or more maintenance doses of 1600 mg of the antibody, or antigen-binding fragment thereof, independently administered to the subject at a frequency of every two weeks.

In some embodiments, the multiple myeloma is relapsed or refractory multiple myeloma (RRMM). In some embodiments, the subject was previously administered one or more therapeutic agents or treatments for multiple myeloma. The one or more previously administered therapeutic agents or treatments for multiple myeloma include, but are not limited to, a proteasome inhibitor (PI), an immunomodulatory drug (IMiD), and an anti-CD38 antibody. In some embodiments, the one or more previously administered therapeutic agents or treatments were not effective in treating the multiple myeloma. In some embodiments, the subject has one or more (e.g., two, three, or four) of: a serum monoclonal paraprotein (M-protein) level of ≥0.5 g/dL, a urine M-protein level of ≥200 mg/24 hours, a serum immunoglobulin free light chain ≥10 mg/dL and/or an abnormal serum immunoglobulin kappa to lambda free light chain ratio.

In some embodiments, these methods result in, e.g., one or more of: a therapeutically desired steady-state concentration of an anti-BCMA antibody in the serum of a subject, a therapeutically desired reduction in the steady-state levels of free light chain in the serum of a subject, and a therapeutically desired saturation of BCMA in a subject.

As described in greater detail in the examples below, initial results from a clinical trial conducted with an afucosylated anti-BCMA antibody such as the SEA-BCMA antibody described herein surprisingly show that the SEA-BCMA antibody can be administered at high doses (e.g., 800 mg or 1600 mg per dose) while still maintaining a tolerable safety profile. These initial results further indicate that such an antibody can potentially be administered in flexible dosing regimens, including standard or intensive dosing regimens. The ability to dose at a high level also indicates that the antibody is potentially a good candidate for dosing in combination with other therapeutic agents, including, for example, dexamethasone.

### Multiple Myeloma

Multiple Myeloma (MM) is a neoplastic disorder of clonally proliferating plasma cells in the bone marrow, peripheral blood, or other extramedullary sites. Diagnosis of MM requiring systemic therapy is defined by International Myeloma Working Group (IMWG) 2014 criteria (Rajkumar, et al. (2014) Lancet Oncol, 15(12):e538-48). Malignant plasma cells exert a direct pathologic effect on the marrow microenvironment and adjacent skeletal bone, leading to anemia, osteolytic bone lesions, and hypercalcemia. In most cases, malignant plasma cells also produce an abnormal monoclonal immunoglobulin known as the M protein, but in a minority of patients, the myeloma cells produce only monoclonal free light chains (FLC). Abnormal levels of either M protein or FLC can contribute to the clinical spectrum of disease that includes renal failure and an increased susceptibility to infections.

Conventional therapy of multiple myeloma (MM), such as combination chemotherapy regimens is not curative and most of the patients ultimately progress. In addition, some patients will not respond to initial treatment.

Duration of initial disease response remains one of the strongest prognostic factors in MM, particularly post autologous stem cell transplantation (ASCT). Early relapse (<24 months) after upfront ASCT strongly predicts lower overall survival (OS), and despite all advancements in the last two decades, the natural history of the disease remains grossly unchanged with the proportion of early relapses stable at around 35-38% (see, Kumar et al., Leukemia 32:986-95, 2018). These relapses usually present aggressively, with similar dismal outcomes from refractory disease, defined as progression under treatment or within 60 days after treatment cessation. Early relapses also do not allow for proper patient recovery from initial treatments and can severely limit treatment choices.

Almost all, if not all, myeloma patients eventually relapse, but while early relapses are usually aggressive and dismal, late relapses (>24 months) generally have a more indolent course. In addition, patients would usually have had time to recover, with little residual toxicity from previous interventions allowing more aggressive approaches. A high unmet need remains in later lines of therapy. The unmet need is pronounced in patients who are refractory to previous administered treatments of PIs, IMiDs, and anti-CD38 antibodies (“triple-class” refractory subjects).

Provided herein are methods of treating a subject having a multiple myeloma (MM). In some embodiments, the multiple myeloma is selected from the group consisting of a precursor to myeloma, multiple myeloma cancers which produce light chains of kappa-type and/or light chains of lambda-type, aggressive multiple myeloma, refractory multiple myeloma, and drug-resistant multiple myeloma. In some embodiments, the multiple myeloma is a relapsed or refractory multiple myeloma (RRMM). In some embodiments, the subject has one or more (e.g., two, three, or four) of: a serum monoclonal paraprotein (M-protein) level of ≥0.5 g/dL, a urine M-protein level of ≥200 mg/24 hours, a serum immunoglobulin free light chain ≥10 mg/dL, and/or an abnormal serum immunoglobulin kappa to lambda free light chain ratio.

Methods for assessing the efficacy of treatment in a subject having multiple myeloma include the measurement of free light chain, M protein, the level of hypercalcemia, and the relative number of myeloma cells in the subject.

### BCMA

B-cell maturation antigen (BCMA or BCM), also known as tumor necrosis factor receptor superfamily member 17 (TNFRSF17), is a protein that in humans is encoded by the TNFRSF17 gene. BCMA is an established plasmablast- and plasma cell-specific protein that mediates cell proliferation and survival. BCMA is expressed at moderate to low levels on the majority of MM patient tumor cells (Novak et al., Blood 103(2):689-694, 2004; Seckinger et al., Cancer Cell 31(3):396-410, 2017). The ligands APRIL and BAFF bind to BCMA and mediate pro-survival cellular signals (Moreaux et al., Blood 103(8):3148-3157, 2004; Novak et al., Blood 103(2):689-694, 2004; O'Connor et al., J. Exp. Med. 199(1):91-8, 2004).

Unless otherwise indicated, BCMA means a human BCMA. Exemplary sequences for wildtype human BCMA protein and wildtype human BCMA cDNA are shown below.

Wildtype Mature Human BCMA Protein
(SEQ ID NO: 9)
MLQMAGQCSQNEYFDSLLHACIPCQLRCSSNTPP
LTCQRYCNASVTNSVKGTNAILWTCLGLSLIIS
LAVFVLMFLLRKINSEPLKDEFKNTGSGLLGMA
NIDLEKSRTGDEIILPRGLEYTVEECTCEDCIK
SKPKVDSDHCFPLPAMEEGATILVTTKTNDYCK
SLPAALSATEIEKSISAR
Wildtype Human BCMA cDNA
(SEQ ID NO: 10)
aagactcaaa cttagaaact tgaattagat
gtggtattca aatccttagc tgccgcgaag
acacagacag cccccgtaag aacccacgaa
gcaggcgaag ttcattgttc tcaacattct
agctgctctt gctgcatttg ctctggaatt
cttgtagaga tattacttgt ccttccaggc
tgttctttct gtagctccct tgttttcttt
ttgtgatcat gttgcagatg gctgggcagt
gctcccaaaa tgaatatttt gacagtttgt
tgcatgcttg cataccttgt caacttcgat
gttcttctaa tactcctcct ctaacatgtc
agcgttattg taatgcaagt gtgaccaatt
cagtgaaagg aacgaatgcg attctctgga
cctgtttggg actgagctta ataatttctt
tggcagtttt cgtgctaatg tttttgctaa
ggaagataaa ctctgaacca ttaaaggacg
agtttaaaaa cacaggatca ggtctcctgg
gcatggctaa cattgacctg gaaaagagca
ggactggtga tgaaattatt cttccgagag
gcctcgagta cacggtggaa gaatgcacct
gtgaagactg catcaagagc aaaccgaagg
tcgactctga ccattgcttt ccactcccag
ctatggagga aggcgcaacc attcttgtca
ccacgaaaac gaatgactat tgcaagagcc
tgccagctgc tttgagtgct acggagatag
agaaatcaat ttctgctagg taattaacca
tttcgactcg agcagtgcca ctttaaaaat
cttttgtcag aatagatgat gtgtcagatc
tctttaggat gactgtattt ttcagttgcc
gatacagctt tttgtcctct aactgtggaa
actctttatg ttagatatat ttctctaggt
tactgttggg agcttaatgg tagaaacttc
cttggtttca tgattaaact cttttttttc
ctga

Unless otherwise apparent from the context reference to BMCA means at least an extracellular domain of a BCMA protein. An exemplary extracellular domain of human BCMA protein comprises amino acids 1 to 54 of SEQ ID NO: 9). In some embodiments, the anti-BCMA antibody or antigen-binding fragment described herein can bind specifically to BCMA expressed on the surface of a cancer cell (e.g., myeloma cell).

### Antibodies and Antigen-Binding Fragments

The term “antibody” is used herein in its broadest sense and includes proteins (e.g., single-chain polypeptides or multi-chain polypeptides) that comprise one or more antigen-binding domains that specifically bind to an antigen or epitope. An intact antibody usually comprises four polypeptides—two heavy chains and two light chains that are joined to form a “Y” shaped molecule. The amino acid sequence in the tips of the “Y” varies greatly among different antibodies. This variable region, composed of, for example, 110-130 amino acids, give the antibody its specificity for binding antigen. The variable region includes the ends of the light and heavy chains. Treating the antibody with a protease can cleave this region, producing Fab or antigen-binding fragment that include the variable ends of an antibody. The regions in the variable region that directly contact a portion of the antigen's surface are complementarity determining regions (CDRs). The light chain variable region (VL) and heavy chain variable region (VH) each comprises three CDRs—CDR1, CDR2, and CDR3. The constant region determines the mechanism used to destroy antigen. Antibodies are divided into five major classes, IgM, IgG, IgA, IgD, and IgE, based on their constant region structure and immune function.

In some embodiments, an antibody specifically includes, e.g., intact antibodies (e.g., intact immunoglobulins, e.g., human IgG (e.g., human IgG1, human IgG2, human IgG3, human IgG4)) and antigen-binding antibody fragments. In some embodiments, the antibody is an humanized IgG1 antibody. One example of an antigen-binding domain is an antigen-binding domain formed by a VH-VL dimer. Additional examples of an antibody are described herein. Additional examples of an antibody are known in the art.

As used herein, the term “antigen-binding domain,” or “antigen-binding fragment” is one or more protein domain(s) (e.g., formed from amino acids from a single polypeptide or formed from amino acids from two or more polypeptides (e.g., the same or different polypeptides)) that is capable of specifically binding to one or more different antigen(s). In some examples, an antigen-binding domain can bind to an antigen or epitope with specificity and affinity similar to that of naturally-occurring antibodies. In some embodiments, an antigen-binding domain can include an alternative scaffold. Non-limiting examples of antigen-binding domains are described herein. Additional examples of antigen-binding domains are known in the art. In some examples, an antigen-binding domain can bind to a single antigen. In some embodiments, the antibody, or antigen-binding fragments used in the methods described herein specifically binds to a B cell maturation antigen (BCMA).

An antibody or antigen-binding fragment thereof described herein can be a single polypeptide, or can comprise two, three, four, five, six, seven, eight, nine, or ten (the same or different) polypeptides. In some embodiments where the antibody or antigen-binding fragment thereof is a single polypeptide, the antibody or antigen-binding fragment can comprise a single antigen-binding domain or two antigen-binding domains. In some embodiments where the antibody or antigen-binding fragment is a single polypeptide and comprises two antigen-binding domains, the first and second antigen-binding domains can be identical or different from each other (and can specifically bind to the same or different antigens or epitopes).

In some embodiments where the antibody or the antigen-binding fragment is a single polypeptide, the first antigen-binding domain and the second antigen-binding domain (if present) can each be independently selected from the group of: a VH domain, a VHH domain, a VNAR domain, and a scFv. In some embodiments where the antibody or the antigen-binding fragment is a single polypeptide, the antibody or antigen-binding fragment can be a BiTe, a (scFv)₂, a nanobody, a nanobody-HSA, a DART, a TandAb, a scDiabody, a scDiabody-CH3, scFv-CH-CL-scFv, a HSAbody, scDiabody-HAS, a tandem-scFv, an Adnectin, a DARPin, a fibronectin, and a DEP conjugate. Additional examples of antigen-binding domains that can be used when the antibody or antigen-binding fragment is a single polypeptide are known in the art.

A VHH domain is a single monomeric variable antibody domain that can be found in camelids. A VNAR domain is a single monomeric variable antibody domain that can be found in cartilaginous fish. Non-limiting aspects of VHH domains and VNAR domains are described in, e.g., Cromie et al., Curr. Top. Med. Chem. 15:2543-2557, 2016; De Genst et al., Dev. Comp. Immunol. 30:187-198, 2006; De Meyer et al., Trends Biotechnol. 32:263-270, 2014; Kijanka et al., Nanomedicine 10:161-174, 2015; Kovaleva et al., Expert. Opin. Biol. Ther. 14:1527-1539, 2014; Krah et al., Immunopharmacol. Immunotoxicol. 38:21-28, 2016; Mujic-Delic et al., Trends Pharmacol. Sci. 35:247-255, 2014; Muyldermans, J. Biotechnol. 74:277-302, 2001; Muyldermans et al., Trends Biochem. Sci. 26:230-235, 2001; Muyldermans, Ann. Rev. Biochem. 82:775-797, 2013; Rahbarizadeh et al., Immunol. Invest. 40:299-338, 2011; Van Audenhove et al., EBioMedicine 8:40-48, 2016; Van Bockstaele et al., Curr Opin. Investig. Drugs 10:1212-1224, 2009; Vincke et al., Methods Mol. Biol. 911:15-26, 2012; and Wesolowski et al., Med. Microbiol. Immunol. 198:157-174, 2009.

In some embodiments where the antibody or antigen-binding fragment is a single polypeptide and comprises two antigen-binding domains, the first antigen-binding domain and the second antigen-binding domain can both be VHH domains, or at least one antigen-binding domain can be a VHH domain. In some embodiments where the antibody or antigen-binding fragment is a single polypeptide and comprises two antigen-binding domains, the first antigen-binding domain and the second antigen-binding domain are both VNAR domains, or at least one antigen-binding domain is a VNAR domain. In some embodiments where the antibody or antigen-binding domain is a single polypeptide, the first antigen-binding domain is a scFv domain. In some embodiments where the antibody or antigen-binding fragment is a single polypeptide and comprises two antigen-binding domains, the first antigen-binding domain and the second antigen-binding domain can both be scFv domains, or at least one antigen-binding domain can be a scFv domain.

In some embodiments, the antibody or antigen-binding fragment can comprise two or more polypeptides (e.g., two, three, four, five, six, seven, eight, nine, or ten polypeptides). In some embodiments where the antibody or antigen-binding fragment comprises two or more polypeptides, two, three, four, five or six of the polypeptides of the two or more polypeptides can be identical.

In some embodiments where the antibody or antigen-binding fragment comprises two or more polypeptides (e.g., two, three, four, five, six, seven, eight, nine, or ten polypeptides), two or more of the polypeptides of the antibody or antigen-binding fragment can assemble (e.g., non-covalently assemble) to form one or more antigen-binding domains, e.g., an antigen-binding fragment of an antibody (e.g., any of the antigen-binding fragments of an antibody described herein), a VHH-scAb, a VHH-Fab, a Dual scFab, a F(ab′)₂, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a KX-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)₂-scFv2, a scFv-KIH, a Fab-scFv-Fc, a tetravalent HCAb, a scDiabody-Fc, a Diabody-Fc, a tandem scFv-Fc, a VHH-Fc, a tandem VHH-Fc, a VHH-Fc KiH, a Fab-VHH-Fe, an Intrabody, a dock and lock, an ImmTAC, an IgG-IgG conjugate, a Cov-X-Body, a scFv1-PEG-scFv2, an Adnectin, a DARPin, a fibronectin, and a DEP conjugate. See, e.g., Spiess et al., Mol. Immunol. 67:95-106, 2015, incorporated in its entirety herewith, for a description of these elements. Non-limiting examples of an antigen-binding fragment of an antibody include an Fv fragment, a Fab fragment, a F(ab′)₂ fragment, and a Fab′ fragment. Additional examples of an antigen-binding fragment of an antibody is an antigen-binding fragment of an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); an antigen-binding fragment of an IgA (e.g., an antigen-binding fragment of IgA1 or IgA2) (e.g., an antigen-binding fragment of a human or humanized IgA, e.g., a human or humanized IgA1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); or an antigen-binding fragment of an IgM (e.g., an antigen-binding fragment of a human or humanized IgM).

A “Fv” fragment comprises a non-covalently-linked dimer of one heavy chain variable domain and one light chain variable domain.

A “Fab” fragment comprises the constant domain of the light chain and the first constant domain (CHI) of the heavy chain, in addition to the heavy and light chain variable domains of the Fv fragment.

A “F(ab′)₂” fragment comprises two Fab fragments joined, near the hinge region, by disulfide bonds.

A “dual variable domain immunoglobulin” or “DVD-Ig” refers to multivalent and multispecific binding proteins as described, e.g., in DiGiammarino et al., Methods Mol. Biol. 899:145-156, 2012; Jakob et al., MABs 5:358-363, 2013; and U.S. Pat. Nos. 7,612,181; 8,258,268; 8,586,714; 8,716,450; 8,722,855; 8,735,546; and 8,822,645, each of which is incorporated by reference in its entirety.

DARTs are described in, e.g., Garber, Nature Reviews Drug Discovery 13:799-801, 2014.

Afucosylated, or non-fucosylated, monoclonal antibodies are monoclonal antibodies engineered so that the oligosaccharides in the Fc region of the antibody do not have any fucose sugar units. In some embodiments, afucosylation of antibodies increases effects such as antibody-dependent cellular cytotoxicity (ADCC). As described in greater detail below, in some embodiments, the antibodies used in the methods described herein are afucosylated antibodies.

In some embodiments, an antibody described herein can be an IgG1 (e.g., human or humanized IgG1), IgG2 (e.g., human or humanized IgG2), IgG3 (e.g., human or humanized IgG3), IgG4 (e.g., human or humanized IgG4), IgA1 (e.g., human or humanized IgA1), IgA2 (e.g., human or humanized IgA2), IgD (e.g., human or humanized IgD), IgE (e.g., human or humanized IgE), or IgM (e.g., human or humanized IgM).

A humanized antibody is a genetically engineered antibody in which CDRs from a non-human “donor” antibody are grafted into human “acceptor” antibody sequences (see, e.g., Queen, U.S. Pat. Nos. 5,530,101 and 5,585,089; Winter, U.S. Pat. No. 5,225,539; Carter, U.S. Pat. No. 6,407,213; Adair, U.S. Pat. No. 5,859,205; and Foote, U.S. Pat. No. 6,881,557). The acceptor antibody sequences can be, for example, a mature human antibody sequence, a composite of such sequences, a consensus sequence of human antibody sequences, or a germline region sequence. For humanization, an exemplary acceptor sequence for the heavy chain is the germline VH exon VH1-2 and for the J exon (JH), exon JH-3. For the light chain, an exemplary acceptor sequence is exon VL1-12 and J exon JK5.

Thus, a humanized antibody is an antibody having at least four CDRs entirely or substantially from a non-human donor antibody and variable region framework sequences and constant regions, if present, entirely or substantially from human antibody sequences. Similarly a humanized heavy chain has at least two and usually all three CDRs entirely or substantially from a donor antibody heavy chain, and a heavy chain variable region framework sequence and heavy chain constant region, if present, substantially from human heavy chain variable region framework and constant region sequences. Similarly a humanized light chain has at least two and usually all three CDRs entirely or substantially from a donor antibody light chain, and a light chain variable region framework sequence and light chain constant region, if present, substantially from human light chain variable region framework and constant region sequences. Other than nanobodies and dAbs, a humanized antibody comprises a humanized heavy chain and a humanized light chain. A CDR in a humanized or human antibody is substantially from or substantially identical to a corresponding CDR in a non-human antibody when at least 60%, 85%, 90%, 95% or 100% of corresponding residues (as defined by Kabat) are identical between the respective CDRs. The variable region framework sequences of an antibody chain or the constant region of an antibody chain are substantially from a human variable region framework sequence or human constant region respectively when at least 70%, 80%, 85%, 90%, 95% or 100% of corresponding residues defined by Kabat are identical.

Although humanized antibodies often incorporate all six CDRs (as defined by Kabat) from a mouse antibody, they can also be made with less than all CDRs (e.g., at least 4 or 5) CDRs from a mouse antibody (e.g., Pascalis et al., J. Immunol. 169:3076, 2002; Vajdos et al., J. Mol. Biol. 320:415-428, 2002; Iwahashi et al., Mol. Immunol. 36:1079-1091, 1999; Tamura et al., J. Immunol. 164:1432-1441, 2000).

Certain amino acids from the human variable region framework residues can be selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

For example, when an amino acid differs between a murine variable region framework residue and a selected human variable region framework residue, the human framework amino acid can be substituted by the equivalent framework amino acid from the mouse antibody when it is reasonably expected that the amino acid:

(1) noncovalently binds antigen directly,

(2) is adjacent to a CDR region,

(3) otherwise interacts with a CDR region (e.g. is within about 6 Å of a CDR region); or

(4) mediates interaction between the heavy and light chains.

In some embodiments of any of the antibodies or antigen-binding fragments described herein, the antibody or antigen-binding fragment can comprise a heavy chain variable region comprising a CDR1 comprising DYYIH (SEQ ID NO: 1), a CDR2 comprising YINPNSGYTNYAQKFQG (SEQ ID NO: 2), and a CDR3 comprising YMWERVTGFFDF (SEQ ID NO: 3), and a light chain variable region comprising a CDR1 comprising LASEDISDDLA (SEQ ID NO: 5), a CDR2 comprising TTSSLQS (SEQ ID NO: 6), and a CDR3 comprising QQTYKFPPT (SEQ ID NO: 7).

In some embodiments of any of the antibodies or antigen-binding fragments described herein, the antibody or antigen-binding fragment can comprise a heavy chain variable region comprising a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 4, and/or a light chain variable domain comprising a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 8.

In some embodiments of any of the antibodies or antigen-binding fragments described herein, the antibody or antigen-binding fragment can comprise a heavy chain variable region encoded by a nucleic acid comprising a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 11, and/or a light chain variable domain encoded by a nucleic acid comprising a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 12.

Exemplary Heavy Chain Variable Domain
(SEQ ID NO: 4)
QVQLVQSGAEVKKPGASVKLSCKASGYTFTDY
YIHWVRQAPGQGLEWIGYINPNSGYTNYAQKF
QGRATMTADKSINTAYVELSRLRSDDTAVYFC
TRYMWERVTGFFDFWGQGTMVTVSS
DNA Encoding Exemplary Heavy Chain
Variable Domain
(SEQ ID NO: 11)
caagtgcagc tggtgcagtc cggagcggaa
gtgaagaaac ctggggcgtc cgtgaagctc
agctgcaagg cctccggcta cactttcacc
gattactaca tccactgggt cagacaggca
ccgggacagg gactggagtg gattggttac
atcaacccca actccgggta caccaattac
gcccagaagt tccagggtcg ggctacgatg
accgccgaca agtcgatcaa cactgcctac
gtggaactgt caaggctgcg gtccgatgac
accgccgtgt acttctgtac ccgctatatg
tgggagcgcg tgactggatt tttcgacttc
tggggccaag gcaccatggt caccgtgtcg
agc
Exemplary Light Chain Variable Domain
(SEQ ID NO: 8)
DIQMTQSPSSVSASVGDRVTITCLASEDISD
DLAWYQQKPGKAPKVLVYTTSSLQSGVPSRF
SGSGSGTDFTLTISSLQPEDFATYFCQQTYK
FPPTFGGGTKVEIKR
DNA Encoding Exemplary Light
Chain Variable Domain
(SEQ ID NO: 12)
gacattcaga tgacccagtc cccctcgtcc
gtgtccgctt ccgtgggaga tcgcgtgacc
atcacttgtc ttgcgtccga ggatatctca
gacgacctgg cctggtacca gcagaagcct
ggaaaggccc cgaaggtcct ggtgtacact
accagcagcc tccagtcggg cgtgccttca
cggttctccg gttcggggtc tggcaccgac
ttcaccctga ctattagctc cctgcaaccc
gaggacttcg ccacctactt ttgccagcaa
acctacaagt tcccgccaac gttcggaggg
ggcaccaagg tcgaaatcaa acgt

In some embodiments of any of the antibodies or antigen-binding fragments described herein, the antibody or antigen-binding fragment can comprise a heavy chain comprising a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 13, and/or a light chain comprising a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 15.

In some embodiments of any of the antibodies or antigen-binding fragments described herein, the antibody or antigen-binding fragment can comprise a heavy chain encoded by a nucleic acid comprising a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 14, and/or a light chain encoded by a nucleic acid comprising a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 16.

Exemplary Heavy Chain
(SEQ ID NO: 13)
QVQLVQSGAEVKKPGASVKLSCKASGYTFTDYYIH
WVRQAPGQGLEWIGYINPNSGYTNYAQKFQGRATM
TADKSINTAYVELSRLRSDDTAVYFCTRYMWERVT
GFFDFWGQGTMVTVSSASTKGPSVFPLAPSSKSTS
GGTAALGCLVKDYFPEPVTVSWNSGALTSGVHTFP
AVLQSSGLYSLSSVVTVPSSSLGTQTYICNVNHKP
SNTKVDKKVEPKSCDKTHTCPPCPAPELLGGPSVF
LFPPKPKDTLMISRTPEVTCVVVDVSHEDPEVKFN
WYVDGVEVHNAKTKPREEQYNSTYRVVSVLTVLHQ
DWLNGKEYKCKVSNKALPAPIEKTISKAKGQPREP
QVYTLPPSRDELTKNQVSLTCLVKGFYPSDIAVEW
ESNGQPENNYKTTPPVLDSDGSFFLYSKLTVDKSR
WQQGNVFSCSVMHEALHNHYTQKSLSLSPGK
DNA Encoding Exemplary Heavy Chain
(SEQ ID NO: 14)
caagtgcagc tggtgcagtc cggagcggaa
gtgaagaaac ctggggcgtc cgtgaagctc
agctgcaagg cctccggcta cactttcacc
gattactaca tccactgggt cagacaggca
ccgggacagg gactggagtg gattggttac
atcaacccca actccgggta caccaattac
gcccagaagt tccagggtcg ggctacgatg
accgccgaca agtcgatcaa cactgcctac
gtggaactgt caaggctgcg gtccgatgac
accgccgtgt acttctgtac ccgctatatg
tgggagcgcg tgactggatt tttcgacttc
tggggccaag gcaccatggt caccgtgtcg
agcgctagca ccaagggccc atcggtcttc
cccctggcac cctcctccaa gagcacctct
gggggcacag cggccctggg ctgcctggtc
aaggactact tccccgaacc ggtgacggtg
tcgtggaact caggcgccct gaccagcggc
gtgcacacct tcccggccgt cctacagtcc
tcaggactct actccctcag cagcgtggtg
accgtgccct ccagcagctt gggcacccag
acctacatct gcaacgtgaa tcacaagccc
agcaacacca aggtggacaa gaaggttgag
cccaaatctt gtgacaaaac tcacacatgc
ccaccgtgcc cagcacctga actcctgggg
ggaccgtcag tcttcctctt ccccccaaaa
cccaaggaca ccctcatgat ctcccggacc
cctgaggtca catgcgtggt ggtggacgtg
agccacgaag accctgaggt caagttcaac
tggtacgtgg acggcgtgga ggtgcataat
gccaagacaa agccgcggga ggagcagtac
aacagcacgt accgtgtggt cagcgtcctc
accgtcctgc accaggactg gctgaatggc
aaggagtaca agtgcaaggt ctccaacaaa
gccctcccag cccccatcga gaaaaccatc
tccaaagcca aagggcagcc ccgagaacca
caggtgtaca ccctgccccc atcccgggac
gagctgacca agaaccaggt cagcctgacc
tgcctggtca aaggcttcta tcccagcgac
atcgccgtgg agtgggagag caatgggcag
ccggagaaca actacaagac cacgcctccc
gtgctggact ccgacggctc cttcttcctc
tacagcaagc tcaccgtgga caagagcagg
tggcagcagg ggaacgtctt ctcatgctcc
gtgatgcatg aggctctgca caaccactac
acgcagaaga gcctctccct gtctccgggt
aaa
Exemplary Light Chain
(SEQ ID NO: 15)
DIQMTQSPSSVSASVGDRVTITCLASEDISDDLA
WYQQKPGKAPKVLVYTTSSLQSGVPSRFSGSGSG
TDFTLTISSLQPEDFATYFCQQTYKFPPTFGGGT
KVEIKRTVAAPSVFIFPPSDEQLKSGTASVVCLL
NNFYPREAKVQWKVDNALQSGNSQESVTEQDSKD
STYSLSSTLTLSKADYEKHKVYACEVTHQGLSSP
VTKSFNRGEC
DNA Encoding Exemplary Light Chain
(SEQ ID NO: 16)
gacattcaga tgacccagtc cccctcgtcc
gtgtccgctt ccgtgggaga tcgcgtgacc
atcacttgtc ttgcgtccga ggatatctca
gacgacctgg cctggtacca gcagaagcct
ggaaaggccc cgaaggtcct ggtgtacact
accagcagcc tccagtcggg cgtgccttca
cggttctccg gttcggggtc tggcaccgac
ttcaccctga ctattagctc cctgcaaccc
gaggacttcg ccacctactt ttgccagcaa
acctacaagt tcccgccaac gttcggaggg
ggcaccaagg tcgaaatcaa acgtacggtg
gctgcaccat ctgtcttcat cttcccgcca
tctgatgagc agttgaaatc tggaactgcc
tctgttgtgt gcctgctgaa taacttctat
cccagagagg ccaaagtaca gtggaaggtg
gataacgccc tccaatcggg taactcccag
gagagtgtca cagagcagga cagcaaggac
agcacctaca gcctcagcag caccctgacg
ctgagcaaag cagactacga gaaacacaaa
gtctacgcct gcgaagtcac ccatcagggc
ctgagctcgc ccgtcacaaa gagcttcaac
aggggagagt gt

In some embodiments of any of the antibodies or antigen-binding fragments described herein, the antibody is one as described in US 2017/0233484 (see also WO 2017/143069). In one such embodiment, the antibody or antigen-binding fragment includes the hSG16.17 VH3 antibody, which comprises a heavy chain variable region comprising a CDR1, CDR2 and CDR3 corresponding to SEQ ID NOs: 60-62 respectively, as listed in US 2017/0233484 and WO 2017/143069, and a light chain variable domain comprising CDR1, CDR2 and CDR3 corresponding to SEQ ID NOs: 90-92, respectively, as listed in US 2017/0233484 and WO 2017/143069. The VH and VL domains of hSG16.17 VH3 correspond to SEQ ID NOs: 13 and 19, respectively, as listed in US 2017/0233484 and WO 2017/143069.

Heavy and light chain variable regions of humanized antibodies can be linked to at least a portion of a human constant region. The choice of constant region depends, in part, whether antibody-dependent cell-mediated cytotoxicity, antibody dependent cellular phagocytosis, and/or complement dependent cytotoxicity are desired. For example, human isotopes IgG1 and IgG3 have strong complement-dependent cytotoxicity, human isotype IgG2 weak complement-dependent cytotoxicity, and human IgG4 lacks complement-dependent cytotoxicity. Human IgG1 and IgG3 also induce stronger cell mediated effector functions than human IgG2 and IgG4. Light chain constant regions can be lambda or kappa. Antibodies can be expressed as tetramers containing two light and two heavy chains, as separate heavy chains, light chains, as Fab, Fab′, F(ab′)₂, and Fv, or as single chain antibodies in which heavy and light chain variable domains are linked through a spacer.

One or several amino acids at the amino or carboxy terminus of the light and/or heavy chain, such as the C-terminal lysine of the heavy chain, may be missing or derivatized in a portion or all of the molecules. Substitutions can be made in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. U.S.A. 103:4005, 2006), or to prolong half-life in humans (see, e.g., Hinton et al., J. Biol. Chem. 279:6213, 2004).

Exemplary substitutions include a substitution of a native amino acid to a cysteine residue at amino acid position 234, 235, 237, 239, 267, 298, 299, 326, 330, or 332, preferably an S239C mutation in a human IgG1 heavy chain (numbering is according to the EU index (Kabat, Sequences of Proteins of Immunological Interest (National Institutes of Health, Bethesda, Md., 1987 and 1991); see US 20100158909, which is herein incorporated reference). A heavy chain can include a S239C substitution, with and without a C-terminal lysine. The presence of an additional cysteine residue allows interchain disulfide bond formation. Such interchain disulfide bond formation can cause steric hindrance, thereby reducing the affinity of the Fc region-FcγR binding interaction. The cysteine residue(s) introduced in or in proximity to the Fc region of an IgG constant region can also serve as sites for conjugation to therapeutic agents (i.e., coupling cytotoxic drugs using thiol specific reagents such as maleimide derivatives of drugs. The presence of a therapeutic agent causes steric hindrance, thereby further reducing the affinity of the Fc region-FcγR binding interaction. Other substitutions at any of heavy chain amino acid positions 234, 235, 236 and/or 237 reduce affinity for Fcγ receptors, particularly FcγRI receptor (see, e.g., U.S. Pat. Nos. 6,624,821, 5,624,821.) A preferred combination of heavy chain amino acid substitutions is S239D, A330L and 1332E, which increases the affinity of the Fc domain for FcγRIIIA and consequently increases ADCC.

The in vivo half-life of an antibody can also impact its effector functions. The half-life of an antibody can be increased or decreased to modify its therapeutic activities. FcRn is a receptor that is structurally similar to MHC Class I antigen that non-covalently associates with 02-microglobulin. FcRn regulates the catabolism of IgGs and their transcytosis across tissues (Ghetie and Ward, Annu. Rev. Immunol. 18:739-766, 2000; Ghetie and Ward, Immunol. Res. 25:97-113, 2002). The IgG-FcRn interaction takes place at pH 6.0 (pH of intracellular vesicles) but not at pH 7.4 (pH of blood); this interaction enables IgGs to be recycled back to the circulation (Ghetie and Ward, Ann. Rev. Immunol. 18:739-766, 2000; Ghetie and Ward, Immunol. Res. 25:97-113, 2002). The region on human IgG1 involved in FcRn binding has been mapped (Shields et al., J. Biol. Chem. 276:6591-604, 2001). Alanine substitutions at heavy chain amino acid positions Pro238, Thr256, Thr307, Gln311, Asp312, Glu380, Glu382, or Asn434 of human IgG1 enhance FcRn binding (Shields et al., J. Biol. Chem. 276:6591-604, 2001). IgG1 molecules harboring these substitutions have longer serum half-lives. Consequently, these modified IgG1 molecules may be able to carry out their effector functions, and hence exert their therapeutic efficacies, over a longer period of time compared to unmodified IgG1. Other exemplary substitutions in a heavy chain for increasing binding to FcRn include introduction of a Gln at amino acid position 250 and/or a Leu at amino acid position 428. EU numbering is used for all positions in the constant region.

Oligosaccharides covalently attached to the conserved Asn297 are involved in the ability of the Fc region of an IgG to bind FcγR (Lund et al., J. Immunol. 157:4963-69, 1996; Wright and Morrison, Trends Biotechnol. 15:26-31, 1997). Engineering of this glycoform on IgG can significantly improve IgG-mediated ADCC. Addition of bisecting N-acetylglucosamine modifications (Umana et al., Nat. Biotechnol. 17:176-180, 1999; Davies et al., Biotech. Bioeng. 74:288-94, 2001) to this glycoform or removal of fucose (Shields et al., J. Biol. Chem. 277:26733-40, 2002; Shinkawa et al., J. Biol. Chem. 278:6591-604, 2003; Niwa et al., Cancer Res. 64:2127-33, 2004) from this glycoform are two examples of IgG Fc engineering that improves the binding between IgG Fc and FcγR, thereby enhancing Ig-mediated ADCC activity.

A systemic substitution of solvent-exposed amino acids of human IgG1 Fc region has generated IgG variants with altered FcγR binding affinities (Shields et al., J. Biol. Chem. 276:6591-604, 2001). When compared to parental IgG1, a subset of these variants involving substitutions at Thr256/Ser298, Ser298/Glu333, Ser298/Lys334, or Ser298/Glu333/Lys334 to Ala demonstrate increased in both binding affinity toward FcγR and ADCC activity (Shields et al., J. Biol. Chem. 276:6591-604, 2001; Okazaki et al., J. Mol. Biol. 336:1239-49, 2004).

Complement fixation activity of antibodies (both C1q binding and CDC activity) can be improved by substitutions at Lys326 and Glu333 (Idusogie et al., J. Immunol. 166:2571-2575, 2001). The same substitutions on a human IgG2 backbone can convert an antibody isotype that binds poorly to C1q and is severely deficient in complement activation activity to one that can both bind C1q and mediate CDC (Idusogie et al., J. Immunol. 166:2571-75, 2001). Several other methods have also been applied to improve complement fixation activity of antibodies. For example, the grafting of an 18-amino acid carboxyl-terminal tail piece of IgM to the carboxyl-termini of IgG greatly enhances their CDC activity. This is observed even with IgG4, which normally has no detectable CDC activity (Smith et al., J. Immunol. 154:2226-36, 1995). Also, substituting Ser444 located close to the carboxy-terminal of IgG1 heavy chain with Cys induced tail-to-tail dimerization of IgG1 with a 200-fold increase of CDC activity over monomeric IgG1 (Shopes et al., J. Immunol. 148:2918-22, 1992). In addition, a bispecific diabody construct with specificity for C1q also confers CDC activity (Kontermann et al., Nat. Biotech. 15:629-31, 1997).

Complement activity can be reduced by mutating at least one of the amino acid residues 318, 320, and 322 of the heavy chain to a residue having a different side chain, such as Ala. Other alkyl-substituted non-ionic residues, such as Gly, Ile, Leu, or Val, or such aromatic non-polar residues as Phe, Tyr, Trp and Pro in place of any one of the three residues also reduce or abolish C1q binding. Ser, Thr, Cys, and Met can be used at residues 320 and 322, but not 318, to reduce or abolish C1q binding activity. Replacement of the 318 (Glu) residue by a polar residue may modify but not abolish C1q binding activity. Replacing residue 297 (Asn) with Ala results in removal of lytic activity, but only slightly reduces (about three-fold weaker) affinity for C1q. This alteration destroys the glycosylation site and the presence of carbohydrate that is required for complement activation. Any other substitution at this site also destroys the glycosylation site. The following heavy chain substitutions and any combination thereof also reduce C1q binding: D270A, K322A, P329A, and P311S (see WO 06/036291).

Reference to a human constant region includes a constant region with any natural allotype or any permutation of residues occupying polymorphic positions in natural allotypes. Also, up to 1, 2, 5, or 10 mutations may be present relative to a natural human constant region, such as those indicated above to reduce Fcγ receptor binding or increase binding to FcRN.

### Non-Fucosylated Antibodies or Antigen-Binding Fragments

In some embodiments, any of the antibodies or antigen-binding fragments as described herein have reduced fucosylation or are non-fucosylated and can be utilized in the methods that are provided. For example, in some embodiments, the antibody or antigen-binding fragment has reduced core fucosylation. “Core fucosylation” refers to addition of fucose (“fucosylation”) to N-acetylglucosamine (“GlcNAc”) at the reducing terminal of an N-linked glycan.

A “complex N-glycoside-linked sugar chain” is typically bound to asparagine 297 (according to the number of Kabat). As used herein, the complex N-glycoside-linked sugar chain has a biantennary composite sugar chain, mainly having the following structure:

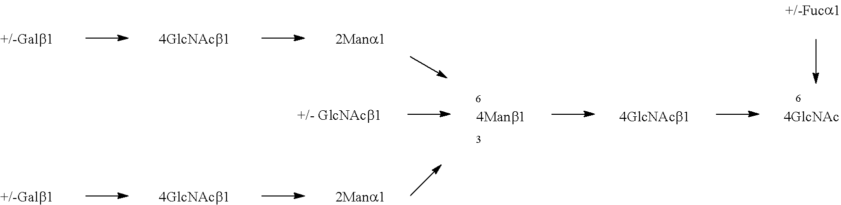

where ± indicates the sugar molecule can be present or absent, and the numbers indicate the position of linkages between the sugar molecules. In the above structure, the sugar chain terminal which binds to asparagine is called a reducing terminal (at right), and the opposite side is called a non-reducing terminal. Fucose is usually bound to N-acetylglucosamine (“GlcNAc”) of the reducing terminal, typically by an α1,6 bond (the 6-position of GlcNAc is linked to the 1-position of fucose). “Gal” refers to galactose, and “Man” refers to mannose.

A “complex N-glycoside-linked sugar chain” includes 1) a complex type, in which the non-reducing terminal side of the core structure has one or more branches of galactose-N-acetylglucosamine (also referred to as “gal-GlcNAc”) and the non-reducing terminal side of Gal-GlcNAc optionally has a sialic acid, bisecting N-acetylglucosamine or the like; or 2) a hybrid type, in which the non-reducing terminal side of the core structure has both branches of a high mannose N-glycoside-linked sugar chain and complex N-glycoside-linked sugar chain. In some embodiments, the “complex N-glycoside-linked sugar chain” includes a complex type in which the non-reducing terminal side of the core structure has zero, one or more branches of galactose-N-acetylglucosamine (also referred to as “gal-GlcNAc”) and the non-reducing terminal side of Gal-GlcNAc optionally further has a structure such as a sialic acid, bisecting N-acetylglucosamine or the like.

In certain embodiments, typically only a minor amount of fucose is incorporated into the complex N-glycoside-linked sugar chain(s) of the antibodies or antigen-binding fragments disclosed herein. For example, in various embodiments, less than about 60%, less than about 50%, less than about 40%, less than about 30%, less than about 20%, less than about 15%, less than about 10%, less than about 5%, or less than about 3% of the molecules of an antibody have core fucosylation by fucose. In some embodiments, about 2% of the molecules of the antibody has core fucosylation by fucose.

In some embodiments, only a minor amount of a fucose analog (or a metabolite or product of the fucose analog) is incorporated into the complex N-glycoside-linked sugar chain(s). For example, in various embodiments, less than about 60%, less than about 50%, less than about 40%, less than about 30%, less than about 20%, less than about 15%, less than about 10%, less than about 5%, or less than about 3% of the antibodies or antigen-binding fragment have core fucosylation by a fucose analog or a metabolite or product of the fucose analog. In some embodiments, about 2% of the antibody or antigen-binding fragment have core fucosylation by a fucose analog or a metabolite or product of the fucose analog.

In some of any of the embodiments disclosed herein, the antibody is an afucosylated antibody, meaning that the antibody at position N297 (EU numbering) does not contain fucose or that a population of such antibodies collectively have no fucose at this position or only have a very low level of fucosylation. For example, in certain embodiments, the antibodies are >90%, or are >95% afucosylated. In some embodiments, the antibodies are at least 95-98% afucosylated, or at least 98-99% afucosylated.

Methods of making non-fucosylated antibodies by incubating antibody-producing cells with a fucose analogue are described, e.g., in WO2009/135181. Briefly, cells that have been engineered to express an antibody or antigen-binding fragment are incubated in the presence of a fucose analogue or an intracellular metabolite or product of the fucose analog. An intracellular metabolite can be, for example, a GDP-modified analog or a fully or partially de-esterified analog. A product can be, for example, a fully or partially de-esterified analog. In some embodiments, a fucose analogue can inhibit an enzyme(s) in the fucose salvage pathway. For example, a fucose analog (or an intracellular metabolite or product of the fucose analog) can inhibit the activity of fucokinase, or GDP-fucose-pyrophosphorylase. In some embodiments, a fucose analog (or an intracellular metabolite or product of the fucose analog) inhibits fucosyltransferase (preferably a 1,6-fucosyltransferase, e.g., the FUT8 protein). In some embodiments, a fucose analog (or an intracellular metabolite or product of the fucose analog) can inhibit the activity of an enzyme in the de novo synthetic pathway for fucose. For example, a fucose analog (or an intracellular metabolite or product of the fucose analog) can inhibit the activity of GDP-mannose 4,6-dehydratase or/or GDP-fucose synthetase. In some embodiments, the fucose analog (or an intracellular metabolite or product of the fucose analog) can inhibit a fucose transporter (e.g., GDP-fucose transporter).

In certain embodiments, the fucose analogue is 2-flurofucose. Methods of using fucose analogues in growth medium and other fucose analogues are disclosed, e.g., in WO/2009/135181.

Other methods for engineering cell lines to reduce core fucosylation included gene knock-outs, gene knock-ins and RNA interference (RNAi). In gene knock-outs, the gene encoding FUT8 (alpha 1,6-fucosyltransferase enzyme) is inactivated. FUT8 catalyzes the transfer of a fucosyl residue from GDP-fucose to position 6 of Asn-linked (N-linked) GlcNac of an N-glycan. FUT8 is reported to be the only enzyme responsible for adding fucose to the N-linked biantennary carbohydrate at Asn297. Gene knock-ins add genes encoding enzymes such as GNTIII or a golgi alpha mannosidase II. An increase in the levels of such enzymes in cells diverts monoclonal antibodies from the fucosylation pathway (leading to decreased core fucosylation), and having increased amount of bisecting N-acetylglucosamines. RNAi typically also targets FUT8 gene expression, leading to decreased mRNA transcript levels or knocking out gene expression entirely. Any of these methods can be used to generate a cell line that would be able to produce a non-fucosylated antibody.

Many methods are available to determine the amount of fucosylation on an antibody. Methods include, e.g., LC-MS via PLRP-S chromatography and electrospray ionization quadrupole TOF MS.

### Production of Antibodies and Antigen-Binding Fragments

Antibodies and antigen-binding fragments are typically produced by recombinant expression. Recombinant polynucleotide constructs typically include an expression control sequence operably linked to the coding sequences of antibody chains, including naturally-associated or heterologous promoter regions. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the produced antibodies or antigen-binding fragments.

Mammalian cells are a preferred host for expressing nucleotide segments encoding antibodies and antigen-binding fragments. See Winnacker, From Genes to Clones, (VCH Publishers, N Y, 1987). A number of suitable host cell lines capable of secreting intact heterologous proteins have been developed in the art, and include CHO cell lines (e.g., DG44), various COS cell lines, HeLa cells, HEK293 cells, L cells, and non-antibody-producing myelomas including Sp2/0 and NS0. Preferably, the cells are nonhuman. Expression vectors for these cells can include expression control sequences, such as an origin of replication, a promoter, an enhancer (Queen et al., Immunol. Rev. 89:49, 1986), and necessary processing information sites, such as ribosome binding sites, RNA splice sites, polyadenylation sites, and transcriptional terminator sequences. Preferred expression control sequences are promoters derived from endogenous genes, cytomegalovirus, SV40, adenovirus, bovine papillomavirus, and the like. See Co et al., J. Immunol. 148:1149, 1992.

Once expressed, antibodies and antigen-binding fragments can be purified according to standard procedures of the art, including HPLC purification, column chromatography, gel electrophoresis and the like (see generally, Scopes, Protein Purification (Springer-Verlag, NY, 1982)).

### Pharmaceutical Compositions

The pharmaceutical compositions used in any of the methods described herein include: (i) an antibody, or antigen-binding fragment thereof, that specifically binds to a B cell maturation antigen (BCMA) (e.g., any of the exemplary antibodies or antigen-binding fragments described herein), and (ii) a pharmaceutically acceptable carrier.

Methods of generating pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral (e.g., intravenous), intradermal, or subcutaneous application can include the following components: a sterile diluent, such as water for injection, saline solution, fixed oils, polyethylene glycols, glycerine, propylene glycol or other synthetic solvents; antibacterial agents, such as benzyl alcohol or methyl parabens; antioxidants, such as ascorbic acid or sodium bisulfite; chelating agents, such as ethylenediaminetetraacetic acid; buffers, such as acetates, citrates, or phosphates; and agents for the adjustment of tonicity, such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.), or phosphate buffered saline (PBS). In some embodiments, the pharmaceutically acceptable carrier is a sodium chloride solution. In all cases, the composition should be sterile. The compositions should be stable under the conditions of manufacture and storage, and must be preserved against the contaminating action of microorganisms, such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating, such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some embodiments, the composition can include isotonic agents, for example, sugars, polyalcohols, such as mannitol, sorbitol, and sodium chloride in the composition. Prolonged absorption of the injectable compositions can be achieved by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the methods of preparation can include the use of vacuum drying and freeze-drying, which yield a powder of the active ingredient, plus any additional desired ingredient from a previously sterile-filtered solution thereof.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### Methods of Treatment

Provided herein are methods of treating a subject having multiple myeloma (MM) that include administering to the subject one or more doses of an antibody, or antigen binding fragment thereof, that specifically binds to a B cell maturation antigen (BCMA) (e.g., any of the exemplary antibodies or antigen-binding fragments described herein).

As used herein, a “subject” typically refers to a human subject, such as a human patient that has multiple myeloma (MM). In some embodiments, the subject has been identified or diagnosed as having a precursor to myeloma, a multiple myeloma cancer which produces light chains of kappa-type and/or light chains of lambda-type, aggressive multiple myeloma, refractory multiple myeloma, or drug-resistant multiple myeloma. In some embodiments, the subject has been identified or diagnosed as having relapsed or refractory multiple myeloma (RRMM). Diagnosis of MM requiring systemic therapy is defined by International Myeloma Working Group (IMWG) 2014 criteria (Rajkumar, et al. (2014) Lancet Oncol, 15(12):e538-48).

In some embodiments, the subject is evaluated to determine if the subject has a small nucleotide polymorphismof FcγRII and/or FcγRIII. In some embodiments, the small nucleotide polymorphisms of FcγRII and FcγRIII may be determined by, for example, testing of the polymorphisms of FCGRIIIA—158V/F, and/or FCGRIIA—131H/R. Accordingly, in some embodiments, the subject has a small nucleotide polymorphism of FcγRII and/or FcγRIII.

In some embodiments, the subject was previously administered one or more therapeutic agents or treatments for multiple myeloma. The one or more previously administered therapeutic agents or treatments for multiple myeloma include, but are not limited to, a proteasome inhibitor (PI), an immunomodulatory drug (IMiD), and an anti-CD38 antibody. In some embodiments, the one or more (e.g., one, two, or three) previously administered therapeutic agents or treatments (e.g., one or more of PIs, IMiDs, and anti-CD38 antibodies) were not effective in treating the multiple myeloma in the subject.

In some embodiments, the subject has one or more of: a serum monoclonal paraprotein (M-protein) level of ≥0.5 g/dL, a urine M-protein level of ≥200 mg/24 hours, a serum immunoglobulin free light chain level of ≥10 mg/dL, and/or an abnormal serum immunoglobulin kappa to lambda free light chain ratio.

In some embodiments, the cancer cells in the subject having MM show detectable levels of BCMA measured at either the protein (e.g., by immunoassay using one of the exemplified antibodies) or mRNA level. In some embodiments, the cancer cells in the subject having MM show elevated levels of BCMA relative to noncancerous tissue of the same type, e.g., from the same or a similar patient. An exemplary level of BCMA on cancer cells can be 5000-150000 BCMA molecules per cell. Optionally, a level of BCMA in a cancer cell from a subject can be measured before administering treatment. In some embodiments, the methods described herein can further include a step of selecting a subject having a multiple myeloma. In some embodiments, specific criteria are applied to the selection of subjects (e.g., any of the inclusion criteria described herein). Such criteria include characteristics of the subjects such as age, gender, the type and stage of a disease, previous treatment history, and other medical conditions.

In some embodiments, the methods described herein can further include terminating the treatment due to the condition of the subject (e.g., using any of the termination criteria described herein).

Also provided herein are methods of excluding a subject from treatment with an anti-BCMA antibody or antigen-binding fragment using one or more of any of the exemplary exclusion criteria described herein.

A. General Dosing

In some embodiments, the one or more doses are independently administered to the subject at about 400 mg of the antibody or antigen-binding fragment to about 2,000 mg of the antibody or antigen-binding fragment (e.g., about 400 mg to about 1,950 mg, about 400 mg to about 1,900 mg, about 400 mg to about 1,850 mg, about 400 mg to about 1,800 mg, about 400 mg to about 1,750 mg, about 400 mg to about 1,700 mg, about 400 mg to about 1,650 mg, about 400 mg to about 1,600 mg, about 400 mg to about 1,550 mg, about 400 mg to about 1,500 mg, about 400 mg to about 1,450 mg, about 400 mg to about 1,400 mg, about 400 mg to about 1,350 mg, about 400 mg to about 1,300 mg, about 400 mg to about 1,250 mg, about 400 mg to about 1,200 mg, about 400 mg to about 1,150 mg, about 400 mg to about 1,100 mg, about 400 mg to about 1,050 mg, about 400 mg to about 1,000 mg, about 400 mg to about 950 mg, about 400 mg to about 900 mg, about 400 mg to about 900 mg, about 400 mg to about 850 mg, about 400 mg to about 800 mg, about 400 mg to about 750 mg, about 400 mg to about 700 mg, about 400 mg to about 650 mg, about 400 mg to about 600 mg, about 400 mg to about 550 mg, about 400 mg to about 500 mg, about 400 mg to about 450 mg, about 500 mg to about 2,000 mg, about 500 mg to about 1,950 mg, about 500 mg to about 1,900 mg, about 500 mg to about 1,850 mg, about 500 mg to about 1,800 mg, about 500 mg to about 1,750 mg, about 500 mg to about 1,700 mg, about 500 mg to about 1,650 mg, about 500 mg to about 1,600 mg, about 500 mg to about 1,550 mg, about 500 mg to about 1,500 mg, about 500 mg to about 1,450 mg, about 500 mg to about 1,400 mg, about 500 mg to about 1,350 mg, about 500 mg to about 1,300 mg, about 500 mg to about 1,250 mg, about 500 mg to about 1,200 mg, about 500 mg to about 1,150 mg, about 500 mg to about 1,100 mg, about 500 mg to about 1,050 mg, about 500 mg to about 1,000 mg, about 500 mg to about 950 mg, about 500 mg to about 900 mg, about 500 mg to about 900 mg, about 500 mg to about 850 mg, about 500 mg to about 800 mg, about 500 mg to about 750 mg, about 500 mg to about 700 mg, about 500 mg to about 650 mg, about 500 mg to about 600 mg, about 500 mg to about 550 mg, about 600 mg to about 2,000 mg, about 600 mg to about 1,950 mg, about 600 mg to about 1,900 mg, about 600 mg to about 1,850 mg, about 600 mg to about 1,800 mg, about 600 mg to about 1,750 mg, about 600 mg to about 1,700 mg, about 600 mg to about 1,650 mg, about 600 mg to about 1,600 mg, about 600 mg to about 1,550 mg, about 600 mg to about 1,500 mg, about 600 mg to about 1,450 mg, about 600 mg to about 1,400 mg, about 600 mg to about 1,350 mg, about 600 mg to about 1,300 mg, about 600 mg to about 1,250 mg, about 600 mg to about 1,200 mg, about 600 mg to about 1,150 mg, about 600 mg to about 1,100 mg, about 600 mg to about 1,050 mg, about 600 mg to about 1,000 mg, about 600 mg to about 950 mg, about 600 mg to about 900 mg, about 600 mg to about 900 mg, about 600 mg to about 850 mg, about 600 mg to about 800 mg, about 600 mg to about 750 mg, about 600 mg to about 700 mg, about 600 mg to about 650 mg, about 700 mg to about 2,000 mg, about 700 mg to about 1,950 mg, about 700 mg to about 1,900 mg, about 700 mg to about 1,850 mg, about 700 mg to about 1,800 mg, about 700 mg to about 1,750 mg, about 700 mg to about 1,700 mg, about 700 mg to about 1,650 mg, about 700 mg to about 1,600 mg, about 700 mg to about 1,550 mg, about 700 mg to about 1,500 mg, about 700 mg to about 1,450 mg, about 700 mg to about 1,400 mg, about 700 mg to about 1,350 mg, about 700 mg to about 1,300 mg, about 700 mg to about 1,250 mg, about 700 mg to about 1,200 mg, about 700 mg to about 1,150 mg, about 700 mg to about 1,100 mg, about 700 mg to about 1,050 mg, about 700 mg to about 1,000 mg, about 700 mg to about 950 mg, about 700 mg to about 900 mg, about 700 mg to about 900 mg, about 700 mg to about 850 mg, about 700 mg to about 800 mg, about 700 mg to about 750 mg, about 800 mg to about 2,000 mg, about 800 mg to about 1,950 mg, about 800 mg to about 1,900 mg, about 800 mg to about 1,850 mg, about 800 mg to about 1,800 mg, about 800 mg to about 1,750 mg, about 800 mg to about 1,700 mg, about 800 mg to about 1,650 mg, about 800 mg to about 1,600 mg, about 800 mg to about 1,550 mg, about 800 mg to about 1,500 mg, about 800 mg to about 1,450 mg, about 800 mg to about 1,400 mg, about 800 mg to about 1,350 mg, about 800 mg to about 1,300 mg, about 800 mg to about 1,250 mg, about 800 mg to about 1,200 mg, about 800 mg to about 1,150 mg, about 800 mg to about 1,100 mg, about 800 mg to about 1,050 mg, about 800 mg to about 1,000 mg, about 800 mg to about 950 mg, about 800 mg to about 900 mg, about 800 mg to about 900 mg, about 800 mg to about 850 mg, about 900 mg to about 2,000 mg, about 900 mg to about 1,950 mg, about 900 mg to about 1,900 mg, about 900 mg to about 1,850 mg, about 900 mg to about 1,800 mg, about 900 mg to about 1,750 mg, about 900 mg to about 1,700 mg, about 900 mg to about 1,650 mg, about 900 mg to about 1,600 mg, about 900 mg to about 1,550 mg, about 900 mg to about 1,500 mg, about 900 mg to about 1,450 mg, about 900 mg to about 1,400 mg, about 900 mg to about 1,350 mg, about 900 mg to about 1,300 mg, about 900 mg to about 1,250 mg, about 900 mg to about 1,200 mg, about 900 mg to about 1,150 mg, about 900 mg to about 1,100 mg, about 900 mg to about 1,050 mg, about 900 mg to about 1,000 mg, about 900 mg to about 950 mg, about 1,000 mg to about 2,000 mg, about 1,000 mg to about 1,950 mg, about 1,000 mg to about 1,900 mg, about 1,000 mg to about 1,850 mg, about 1,000 mg to about 1,800 mg, about 1,000 mg to about 1,750 mg, about 1,000 mg to about 1,700 mg, about 1,000 mg to about 1,650 mg, about 1,000 mg to about 1,600 mg, about 1,000 mg to about 1,550 mg, about 1,000 mg to about 1,500 mg, about 1,000 mg to about 1,450 mg, about 1,000 mg to about 1,400 mg, about 1,000 mg to about 1,350 mg, about 1,000 mg to about 1,300 mg, about 1,000 mg to about 1,250 mg, about 1,000 mg to about 1,200 mg, about 1,000 mg to about 1,150 mg, about 1,000 mg to about 1,100 mg, about 1,000 mg to about 1,050 mg, about 1,100 mg to about 2,000 mg, about 1,100 mg to about 1,950 mg, about 1,100 mg to about 1,900 mg, about 1,100 mg to about 1,850 mg, about 1,100 mg to about 1,800 mg, about 1,100 mg to about 1,750 mg, about 1,100 mg to about 1,700 mg, about 1,100 mg to about 1,650 mg, about 1,100 mg to about 1,600 mg, about 1,100 mg to about 1,550 mg, about 1,100 mg to about 1,500 mg, about 1,100 mg to about 1,450 mg, about 1,100 mg to about 1,400 mg, about 1,100 mg to about 1,350 mg, about 1,100 mg to about 1,300 mg, about 1,100 mg to about 1,250 mg, about 1,100 mg to about 1,200 mg, about 1,100 mg to about 1,150 mg, about 1,200 mg to about 2,000 mg, about 1,200 mg to about 1,950 mg, about 1,200 mg to about 1,900 mg, about 1,200 mg to about 1,850 mg, about 1,200 mg to about 1,800 mg, about 1,200 mg to about 1,750 mg, about 1,200 mg to about 1,700 mg, about 1,200 mg to about 1,650 mg, about 1,200 mg to about 1,600 mg, about 1,200 mg to about 1,550 mg, about 1,200 mg to about 1,500 mg, about 1,200 mg to about 1,450 mg, about 1,200 mg to about 1,400 mg, about 1,200 mg to about 1,350 mg, about 1,200 mg to about 1,300 mg, about 1,200 mg to about 1,250 mg, about 1,300 mg to about 2,000 mg, about 1,300 mg to about 1,950 mg, about 1,300 mg to about 1,900 mg, about 1,300 mg to about 1,850 mg, about 1,300 mg to about 1,800 mg, about 1,300 mg to about 1,750 mg, about 1,300 mg to about 1,700 mg, about 1,300 mg to about 1,650 mg, about 1,300 mg to about 1,600 mg, about 1,300 mg to about 1,550 mg, about 1,300 mg to about 1,500 mg, about 1,300 mg to about 1,450 mg, about 1,300 mg to about 1,400 mg, about 1,300 mg to about 1,350 mg, about 1,400 mg to about 2,000 mg, about 1,400 mg to about 1,950 mg, about 1,400 mg to about 1,900 mg, about 1,400 mg to about 1,850 mg, about 1,400 mg to about 1,800 mg, about 1,400 mg to about 1,750 mg, about 1,400 mg to about 1,700 mg, about 1,400 mg to about 1,650 mg, about 1,400 mg to about 1,600 mg, about 1,400 mg to about 1,550 mg, about 1,400 mg to about 1,500 mg, about 1,400 mg to about 1,450 mg, about 1,500 mg to about 2,000 mg, about 1,500 mg to about 1,950 mg, about 1,500 mg to about 1,900 mg, about 1,500 mg to about 1,850 mg, about 1,500 mg to about 1,800 mg, about 1,500 mg to about 1,750 mg, about 1,500 mg to about 1,700 mg, about 1,500 mg to about 1,650 mg, about 1,500 mg to about 1,600 mg, about 1,500 mg to about 1,550 mg, about 1,600 mg to about 2,000 mg, about 1,600 mg to about 1,950 mg, about 1,600 mg to about 1,900 mg, about 1,600 mg to about 1,850 mg, about 1,600 mg to about 1,800 mg, about 1,600 mg to about 1,750 mg, about 1,600 mg to about 1,700 mg, about 1,600 mg to about 1,650 mg, about 1,700 mg to about 2,000 mg, about 1,700 mg to about 1,950 mg, about 1,700 mg to about 1,900 mg, about 1,700 mg to about 1,850 mg, about 1,700 mg to about 1,800 mg, about 1,700 mg to about 1,750 mg, about 1,800 mg to about 2,000 mg, about 1,800 mg to about 1,950 mg, about 1,800 mg to about 1,900 mg, about 1,800 mg to about 1,850 mg, about 1,900 mg to about 2,000 mg, or about 1,900 mg to about 1,950 mg).

In some embodiments, one or more doses of about 0.01 mg/kg to about 100 mg/kg (e.g., about 0.03 mg/kg to about 50 mg/kg; about 0.1 mg/kg to about 20 mg/kg; about 1 mg/kg to about 10 mg/kg; about 1 mg/kg to about 5 mg/kg; about 0.1 mg/kg to about 5 mg/kg; or about 1 mg/kg to about 2 mg/kg) of the antibody or antigen-binding fragment thereof as described herein is administered to the subject. In some embodiments, the dosage for the antibody or antigen-binding fragment thereof as described herein can be less than 100 mg/kg, less than 50 mg/kg, less than 20 mg/kg, less than 10 mg/kg, less than 9 mg/kg, less than 8 mg/kg, less than 7 mg/kg, less than 6 mg/kg, less than 5 mg/kg, less than 4 mg/kg, less than 3 mg/kg, less than 2 mg/kg, less than 1 mg/kg, less than 0.5 mg/kg, or less than 0.1 mg/kg. In some embodiments, the dosage for the antibody or antigen-binding fragments thereof as described herein can be greater than 50 mg/kg, greater than 20 mg/kg, greater than 10 mg/kg, greater than 9 mg/kg, greater than 8 mg/kg, greater than 7 mg/kg, greater than 6 mg/kg, greater than 5 mg/kg, greater than 4 mg/kg, greater than 3 mg/kg, greater than 2 mg/kg, greater than 1 mg/kg, greater than 0.5 mg/kg, or greater than 0.1 mg/kg.

In some embodiments of any of the methods described herein, the antibody or antigen binding fragment thereof, comprises: a heavy chain variable region comprising a CDR1 comprising SEQ ID NO: 1, a CDR2 comprising SEQ ID NO: 2, and a CDR3 comprising SEQ ID NO: 3, and a light chain variable domain comprising a CDR1 comprising SEQ ID NO: 5, a CDR2 comprising SEQ ID NO: 6, and a CDR3 comprising SEQ ID NO: 7. In some embodiments of any of the methods described herein, the antibody or antigen binding fragment thereof, comprises: a heavy chain variable region comprising SEQ ID NO: 4, and a light chain variable region comprising SEQ ID NO: 8.

In some embodiments, the subject is administered two or more (e.g., 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, or 50 or more) doses of the pharmaceutical composition (e.g., any of the pharmaceutical compositions comprising any of the antibodies or antigen-binding fragments described herein).

In some embodiments, the subject is administered 1 to about 50 doses (e.g., 1 to about 45 doses, 1 to about 40 doses, 1 to about 35 doses, 1 to about 30 doses, 1 to about 25 doses, 1 to about 20 doses, 1 to about 18 doses, 1 to about 16 doses, 1 to about 14 doses, 1 to about 12 doses, 1 to about 10 doses, 1 to about 8 doses, 1 to about 6 doses, 1 to about 4 doses, 1 to about 2 doses, about 2 to about 50 doses, about 2 to about 45 doses, about 2 to about 40 doses, about 2 to about 35 doses, about 2 to about 30 doses, about 2 to about 25 doses, about 2 to about 20 doses, about 2 to about 18 doses, about 2 to about 16 doses, about 2 to about 14 doses, about 2 to about 12 doses, about 2 to about 10 doses, about 2 to about 8 doses, about 2 to about 6 doses, about 2 to about 4 doses, about 4 to about 50 doses, about 4 to about 45 doses, about 4 to about 40 doses, about 4 to about 35 doses, about 4 to about 30 doses, about 4 to about 25 doses, about 4 to about 20 doses, about 4 to about 18 doses, about 4 to about 16 doses, about 4 to about 14 doses, about 4 to about 12 doses, about 4 to about 10 doses, about 4 to about 8 doses, about 4 to about 6 doses, about 6 to about 50 doses, about 6 to about 45 doses, about 6 to about 40 doses, about 6 to about 35 doses, about 6 to about 30 doses, about 6 to about 25 doses, about 6 to about 20 doses, about 6 to about 18 doses, about 6 to about 16 doses, about 6 to about 14 doses, about 6 to about 12 doses, about 6 to about 10 doses, about 6 to about 8 doses, about 8 to about 50 doses, about 8 to about 45 doses, about 8 to about 40 doses, about 8 to about 35 doses, about 8 to about 30 doses, about 8 to about 25 doses, about 8 to about 20 doses, about 8 to about 18 doses, about 8 to about 16 doses, about 8 to about 14 doses, about 8 to about 12 doses, about 8 to about 10 doses, about 10 to about 50 doses, about 10 to about 45 doses, about 10 to about 40 doses, about 10 to about 35 doses, about 10 to about 30 doses, about 10 to about 25 doses, about 10 to about 20 doses, about 10 to about 18 doses, about 10 to about 16 doses, about 10 to about 14 doses, about 10 to about 12 doses, about 12 to about 50 doses, about 12 to about 45 doses, about 12 to about 40 doses, about 12 to about 35 doses, about 12 to about 30 doses, about 12 to about 25 doses, about 12 to about 20 doses, about 12 to about 18 doses, about 12 to about 16 doses, about 12 to about 14 doses, about 14 to about 50 doses, about 14 to about 45 doses, about 14 to about 40 doses, about 14 to about 35 doses, about 14 to about 30 doses, about 14 to about 25 doses, about 14 to about 20 doses, about 14 to about 18 doses, about 14 to about 16 doses, about 16 to about 50 doses, about 16 to about 45 doses, about 16 to about 40 doses, about 16 to about 35 doses, about 16 to about 30 doses, about 16 to about 25 doses, about 16 to about 20 doses, about 16 to about 18 doses, about 18 to about 50 doses, about 18 to about 45 doses, about 18 to about 40 doses, about 18 to about 35 doses, about 18 to about 30 doses, about 18 to about 25 doses, about 19 to about 20 doses, about 20 to about 50 doses, about 20 to about 45 doses, about 20 to about 40 doses, about 20 to about 35 doses, about 20 to about 30 doses, about 20 to about 25 doses, about 25 to about 50 doses, about 25 to about 45 doses, about 25 to about 40 doses, about 25 to about 35 doses, about 25 to about 30 doses, about 30 to about 50 doses, about 30 to about 45 doses, about 30 to about 40 doses, about 30 to about 35 doses, about 35 to about 50 doses, about 35 to about 45 doses, about 35 to about 40 doses, about 40 to about 50 doses, about 40 to about 45 doses, or about 45 to about 50 doses) of the pharmaceutical composition (e.g., any of the pharmaceutical compositions comprising any of the antibodies or antigen-binding fragments described herein).

In some embodiments, where two or more doses of the pharmaceutical composition (e.g., any of the pharmaceutical compositions comprising any of the antibodies or antigen-binding fragments described herein) are administered to the subject (e.g., any of the subjects described herein), any two consecutive doses can be administered at a frequency of about one week to about two months (e.g., about one week to about 7 weeks, about one week to about 6 weeks, about one week to about 5 weeks, about one week to about 4 weeks, about one week to about 3 weeks, about one week to about 2 weeks, about 2 weeks to about two months, about 2 weeks to about 7 weeks, about 2 weeks to about 6 weeks, about 2 weeks to about 5 weeks, about 2 weeks to about 4 weeks, about 2 weeks to about 3 weeks, about 3 weeks to about two months, about 3 weeks to about 7 weeks, about 3 weeks to about 6 weeks, about 3 weeks to about 5 weeks, about 3 weeks to about 4 weeks, about 4 weeks to about 2 months, about 4 weeks to about 7 weeks, about 4 weeks to about 6 weeks, about 4 weeks to about 5 weeks, about 5 weeks to about 2 months, about 5 weeks to about 7 weeks, about 5 weeks to about 6 weeks, about 6 weeks to about 2 months, about 6 weeks to about 7 weeks, or about 7 weeks to about 2 months). In some embodiments, the frequency between any two doses remains the same over a treatment period (period of time between the first administered dose and the last administered dose). In some embodiments, the frequency between any two doses can be varied over a treatment period.

In some embodiments, 100 mg of the antibody or antigen-binding fragment is administered to the subject once every two weeks. In some embodiments, 200 mg of the antibody or antigen-binding fragment is administered to the subject once every two weeks. In some embodiments, 400 mg of the antibody or antigen-binding fragment is administered to the subject once every two weeks. In some embodiments, 800 mg of the antibody or antigen-binding fragment is administered to the subject once every two weeks. In some embodiments, 1600 mg of the antibody or antigen-binding fragment is administered to the subject once every two weeks. In some embodiments, the antibody or antigen-binding fragment can be administered to the subject on Day 1 and Day 15 of repeated 28-day cycles.

B. Induction and Maintenance Dosing

In some embodiments, the methods described herein comprise administering to the subject one or more induction doses of an antibody or an antigen-binding fragment described herein). In some embodiments, the methods described herein further comprise administering to the subject one more maintenance doses of an antibody or an antigen-binding fragment described herein).

In some embodiments, the one or more induction doses are independently administered to the subject at about 100, 200, 400, 800, or 1600 mg of the antibody or antigen-binding fragment. In some embodiments, the one or more induction doses is 800 mg of the antibody or antigen-binding fragment. In further embodiments, the one or more induction doses is 1600 mg of the antibody or antigen-binding fragment.

In certain embodiments, the one or more induction doses are independently administered to the subject at about 400 mg of the antibody or antigen-binding fragment to about 2,000 mg of the antibody or antigen-binding fragment (e.g., about 400 mg to about 1,950 mg, about 400 mg to about 1,900 mg, about 400 mg to about 1,850 mg, about 400 mg to about 1,800 mg, about 400 mg to about 1,750 mg, about 400 mg to about 1,700 mg, about 400 mg to about 1,650 mg, about 400 mg to about 1,600 mg, about 400 mg to about 1,550 mg, about 400 mg to about 1,500 mg, about 400 mg to about 1,450 mg, about 400 mg to about 1,400 mg, about 400 mg to about 1,350 mg, about 400 mg to about 1,300 mg, about 400 mg to about 1,250 mg, about 400 mg to about 1,200 mg, about 400 mg to about 1,150 mg, about 400 mg to about 1,100 mg, about 400 mg to about 1,050 mg, about 400 mg to about 1,000 mg, about 400 mg to about 950 mg, about 400 mg to about 900 mg, about 400 mg to about 900 mg, about 400 mg to about 850 mg, about 400 mg to about 800 mg, about 400 mg to about 750 mg, about 400 mg to about 700 mg, about 400 mg to about 650 mg, about 400 mg to about 600 mg, about 400 mg to about 550 mg, about 400 mg to about 500 mg, about 400 mg to about 450 mg, about 500 mg to about 2,000 mg, about 500 mg to about 1,950 mg, about 500 mg to about 1,900 mg, about 500 mg to about 1,850 mg, about 500 mg to about 1,800 mg, about 500 mg to about 1,750 mg, about 500 mg to about 1,700 mg, about 500 mg to about 1,650 mg, about 500 mg to about 1,600 mg, about 500 mg to about 1,550 mg, about 500 mg to about 1,500 mg, about 500 mg to about 1,450 mg, about 500 mg to about 1,400 mg, about 500 mg to about 1,350 mg, about 500 mg to about 1,300 mg, about 500 mg to about 1,250 mg, about 500 mg to about 1,200 mg, about 500 mg to about 1,150 mg, about 500 mg to about 1,100 mg, about 500 mg to about 1,050 mg, about 500 mg to about 1,000 mg, about 500 mg to about 950 mg, about 500 mg to about 900 mg, about 500 mg to about 900 mg, about 500 mg to about 850 mg, about 500 mg to about 800 mg, about 500 mg to about 750 mg, about 500 mg to about 700 mg, about 500 mg to about 650 mg, about 500 mg to about 600 mg, about 500 mg to about 550 mg, about 600 mg to about 2,000 mg, about 600 mg to about 1,950 mg, about 600 mg to about 1,900 mg, about 600 mg to about 1,850 mg, about 600 mg to about 1,800 mg, about 600 mg to about 1,750 mg, about 600 mg to about 1,700 mg, about 600 mg to about 1,650 mg, about 600 mg to about 1,600 mg, about 600 mg to about 1,550 mg, about 600 mg to about 1,500 mg, about 600 mg to about 1,450 mg, about 600 mg to about 1,400 mg, about 600 mg to about 1,350 mg, about 600 mg to about 1,300 mg, about 600 mg to about 1,250 mg, about 600 mg to about 1,200 mg, about 600 mg to about 1,150 mg, about 600 mg to about 1,100 mg, about 600 mg to about 1,050 mg, about 600 mg to about 1,000 mg, about 600 mg to about 950 mg, about 600 mg to about 900 mg, about 600 mg to about 900 mg, about 600 mg to about 850 mg, about 600 mg to about 800 mg, about 600 mg to about 750 mg, about 600 mg to about 700 mg, about 600 mg to about 650 mg, about 700 mg to about 2,000 mg, about 700 mg to about 1,950 mg, about 700 mg to about 1,900 mg, about 700 mg to about 1,850 mg, about 700 mg to about 1,800 mg, about 700 mg to about 1,750 mg, about 700 mg to about 1,700 mg, about 700 mg to about 1,650 mg, about 700 mg to about 1,600 mg, about 700 mg to about 1,550 mg, about 700 mg to about 1,500 mg, about 700 mg to about 1,450 mg, about 700 mg to about 1,400 mg, about 700 mg to about 1,350 mg, about 700 mg to about 1,300 mg, about 700 mg to about 1,250 mg, about 700 mg to about 1,200 mg, about 700 mg to about 1,150 mg, about 700 mg to about 1,100 mg, about 700 mg to about 1,050 mg, about 700 mg to about 1,000 mg, about 700 mg to about 950 mg, about 700 mg to about 900 mg, about 700 mg to about 900 mg, about 700 mg to about 850 mg, about 700 mg to about 800 mg, about 700 mg to about 750 mg, about 800 mg to about 2,000 mg, about 800 mg to about 1,950 mg, about 800 mg to about 1,900 mg, about 800 mg to about 1,850 mg, about 800 mg to about 1,800 mg, about 800 mg to about 1,750 mg, about 800 mg to about 1,700 mg, about 800 mg to about 1,650 mg, about 800 mg to about 1,600 mg, about 800 mg to about 1,550 mg, about 800 mg to about 1,500 mg, about 800 mg to about 1,450 mg, about 800 mg to about 1,400 mg, about 800 mg to about 1,350 mg, about 800 mg to about 1,300 mg, about 800 mg to about 1,250 mg, about 800 mg to about 1,200 mg, about 800 mg to about 1,150 mg, about 800 mg to about 1,100 mg, about 800 mg to about 1,050 mg, about 800 mg to about 1,000 mg, about 800 mg to about 950 mg, about 800 mg to about 900 mg, about 800 mg to about 900 mg, about 800 mg to about 850 mg, about 900 mg to about 2,000 mg, about 900 mg to about 1,950 mg, about 900 mg to about 1,900 mg, about 900 mg to about 1,850 mg, about 900 mg to about 1,800 mg, about 900 mg to about 1,750 mg, about 900 mg to about 1,700 mg, about 900 mg to about 1,650 mg, about 900 mg to about 1,600 mg, about 900 mg to about 1,550 mg, about 900 mg to about 1,500 mg, about 900 mg to about 1,450 mg, about 900 mg to about 1,400 mg, about 900 mg to about 1,350 mg, about 900 mg to about 1,300 mg, about 900 mg to about 1,250 mg, about 900 mg to about 1,200 mg, about 900 mg to about 1,150 mg, about 900 mg to about 1,100 mg, about 900 mg to about 1,050 mg, about 900 mg to about 1,000 mg, about 900 mg to about 950 mg, about 1,000 mg to about 2,000 mg, about 1,000 mg to about 1,950 mg, about 1,000 mg to about 1,900 mg, about 1,000 mg to about 1,850 mg, about 1,000 mg to about 1,800 mg, about 1,000 mg to about 1,750 mg, about 1,000 mg to about 1,700 mg, about 1,000 mg to about 1,650 mg, about 1,000 mg to about 1,600 mg, about 1,000 mg to about 1,550 mg, about 1,000 mg to about 1,500 mg, about 1,000 mg to about 1,450 mg, about 1,000 mg to about 1,400 mg, about 1,000 mg to about 1,350 mg, about 1,000 mg to about 1,300 mg, about 1,000 mg to about 1,250 mg, about 1,000 mg to about 1,200 mg, about 1,000 mg to about 1,150 mg, about 1,000 mg to about 1,100 mg, about 1,000 mg to about 1,050 mg, about 1,100 mg to about 2,000 mg, about 1,100 mg to about 1,950 mg, about 1,100 mg to about 1,900 mg, about 1,100 mg to about 1,850 mg, about 1,100 mg to about 1,800 mg, about 1,100 mg to about 1,750 mg, about 1,100 mg to about 1,700 mg, about 1,100 mg to about 1,650 mg, about 1,100 mg to about 1,600 mg, about 1,100 mg to about 1,550 mg, about 1,100 mg to about 1,500 mg, about 1,100 mg to about 1,450 mg, about 1,100 mg to about 1,400 mg, about 1,100 mg to about 1,350 mg, about 1,100 mg to about 1,300 mg, about 1,100 mg to about 1,250 mg, about 1,100 mg to about 1,200 mg, about 1,100 mg to about 1,150 mg, about 1,200 mg to about 2,000 mg, about 1,200 mg to about 1,950 mg, about 1,200 mg to about 1,900 mg, about 1,200 mg to about 1,850 mg, about 1,200 mg to about 1,800 mg, about 1,200 mg to about 1,750 mg, about 1,200 mg to about 1,700 mg, about 1,200 mg to about 1,650 mg, about 1,200 mg to about 1,600 mg, about 1,200 mg to about 1,550 mg, about 1,200 mg to about 1,500 mg, about 1,200 mg to about 1,450 mg, about 1,200 mg to about 1,400 mg, about 1,200 mg to about 1,350 mg, about 1,200 mg to about 1,300 mg, about 1,200 mg to about 1,250 mg, about 1,300 mg to about 2,000 mg, about 1,300 mg to about 1,950 mg, about 1,300 mg to about 1,900 mg, about 1,300 mg to about 1,850 mg, about 1,300 mg to about 1,800 mg, about 1,300 mg to about 1,750 mg, about 1,300 mg to about 1,700 mg, about 1,300 mg to about 1,650 mg, about 1,300 mg to about 1,600 mg, about 1,300 mg to about 1,550 mg, about 1,300 mg to about 1,500 mg, about 1,300 mg to about 1,450 mg, about 1,300 mg to about 1,400 mg, about 1,300 mg to about 1,350 mg, about 1,400 mg to about 2,000 mg, about 1,400 mg to about 1,950 mg, about 1,400 mg to about 1,900 mg, about 1,400 mg to about 1,850 mg, about 1,400 mg to about 1,800 mg, about 1,400 mg to about 1,750 mg, about 1,400 mg to about 1,700 mg, about 1,400 mg to about 1,650 mg, about 1,400 mg to about 1,600 mg, about 1,400 mg to about 1,550 mg, about 1,400 mg to about 1,500 mg, about 1,400 mg to about 1,450 mg, about 1,500 mg to about 2,000 mg, about 1,500 mg to about 1,950 mg, about 1,500 mg to about 1,900 mg, about 1,500 mg to about 1,850 mg, about 1,500 mg to about 1,800 mg, about 1,500 mg to about 1,750 mg, about 1,500 mg to about 1,700 mg, about 1,500 mg to about 1,650 mg, about 1,500 mg to about 1,600 mg, about 1,500 mg to about 1,550 mg, about 1,600 mg to about 2,000 mg, about 1,600 mg to about 1,950 mg, about 1,600 mg to about 1,900 mg, about 1,600 mg to about 1,850 mg, about 1,600 mg to about 1,800 mg, about 1,600 mg to about 1,750 mg, about 1,600 mg to about 1,700 mg, about 1,600 mg to about 1,650 mg, about 1,700 mg to about 2,000 mg, about 1,700 mg to about 1,950 mg, about 1,700 mg to about 1,900 mg, about 1,700 mg to about 1,850 mg, about 1,700 mg to about 1,800 mg, about 1,700 mg to about 1,750 mg, about 1,800 mg to about 2,000 mg, about 1,800 mg to about 1,950 mg, about 1,800 mg to about 1,900 mg, about 1,800 mg to about 1,850 mg, about 1,900 mg to about 2,000 mg, or about 1,900 mg to about 1,950 mg). In some embodiments, one or more 1600 mg induction doses of the pharmaceutical composition are independently administered to the subject. In some embodiments, one or more 800 mg induction doses of the pharmaceutical composition are independently administered to the subject.

In some embodiments, the subject is administered 1 to about 50 induction doses (e.g., 1 to about 45 doses, 1 to about 40 doses, 1 to about 35 doses, 1 to about 30 doses, 1 to about 25 doses, 1 to about 20 doses, 1 to about 18 doses, 1 to about 16 doses, 1 to about 14 doses, 1 to about 12 doses, 1 to about 10 doses, 1 to about 8 doses, 1 to about 6 doses, 1 to about 4 doses, 1 to about 2 doses, about 2 to about 50 doses, about 2 to about 45 doses, about 2 to about 40 doses, about 2 to about 35 doses, about 2 to about 30 doses, about 2 to about 25 doses, about 2 to about 20 doses, about 2 to about 18 doses, about 2 to about 16 doses, about 2 to about 14 doses, about 2 to about 12 doses, about 2 to about 10 doses, about 2 to about 8 doses, about 2 to about 6 doses, about 2 to about 4 doses, about 4 to about 50 doses, about 4 to about 45 doses, about 4 to about 40 doses, about 4 to about 35 doses, about 4 to about 30 doses, about 4 to about 25 doses, about 4 to about 20 doses, about 4 to about 18 doses, about 4 to about 16 doses, about 4 to about 14 doses, about 4 to about 12 doses, about 4 to about 10 doses, about 4 to about 8 doses, about 4 to about 6 doses, about 6 to about 50 doses, about 6 to about 45 doses, about 6 to about 40 doses, about 6 to about 35 doses, about 6 to about 30 doses, about 6 to about 25 doses, about 6 to about 20 doses, about 6 to about 18 doses, about 6 to about 16 doses, about 6 to about 14 doses, about 6 to about 12 doses, about 6 to about 10 doses, about 6 to about 8 doses, about 8 to about 50 doses, about 8 to about 45 doses, about 8 to about 40 doses, about 8 to about 35 doses, about 8 to about 30 doses, about 8 to about 25 doses, about 8 to about 20 doses, about 8 to about 18 doses, about 8 to about 16 doses, about 8 to about 14 doses, about 8 to about 12 doses, about 8 to about 10 doses, about 10 to about 50 doses, about 10 to about 45 doses, about 10 to about 40 doses, about 10 to about 35 doses, about 10 to about 30 doses, about 10 to about 25 doses, about 10 to about 20 doses, about 10 to about 18 doses, about 10 to about 16 doses, about 10 to about 14 doses, about 10 to about 12 doses, about 12 to about 50 doses, about 12 to about 45 doses, about 12 to about 40 doses, about 12 to about 35 doses, about 12 to about 30 doses, about 12 to about doses, about 12 to about 20 doses, about 12 to about 18 doses, about 12 to about 16 doses, about 12 to about 14 doses, about 14 to about 50 doses, about 14 to about 45 doses, about 14 to about 40 doses, about 14 to about 35 doses, about 14 to about 30 doses, about 14 to about 25 doses, about 14 to about 20 doses, about 14 to about 18 doses, about 14 to about 16 doses, about 16 to about 50 doses, about 16 to about 45 doses, about 16 to about 40 doses, about 16 to about 35 doses, about 16 to about 30 doses, about 16 to about 25 doses, about 16 to about 20 doses, about 16 to about 18 doses, about 18 to about 50 doses, about 18 to about 45 doses, about 18 to about 40 doses, about 18 to about 35 doses, about 18 to about 30 doses, about 18 to about 25 doses, about 19 to about 20 doses, about 20 to about 50 doses, about 20 to about 45 doses, about 20 to about 40 doses, about 20 to about 35 doses, about 20 to about 30 doses, about 20 to about 25 doses, about 25 to about 50 doses, about 25 to about 45 doses, about 25 to about 40 doses, about 25 to about 35 doses, about 25 to about 30 doses, about 30 to about 50 doses, about 30 to about 45 doses, about 30 to about 40 doses, about 30 to about 35 doses, about 35 to about 50 doses, about 35 to about 45 doses, about 35 to about 40 doses, about 40 to about 50 doses, about 40 to about 45 doses, or about 45 to about 50 doses) of the pharmaceutical composition (e.g., any of the pharmaceutical compositions comprising any of the antibodies or antigen-binding fragments described herein). In some embodiments, the subject is administered about 1 to about 3 induction doses of the pharmaceutical composition (e.g., any of the pharmaceutical compositions comprising any of the antibodies or antigen-binding fragments described herein).

In some embodiments, where two or more maintenance doses of the pharmaceutical composition (e.g., any of the pharmaceutical compositions comprising any of the antibodies or antigen-binding fragments described herein) are administered to the subject (e.g., any of the subjects described herein), any two consecutive induction doses can be administered at a frequency of about one week to about two months (e.g., about one week to about 7 weeks, about one week to about 6 weeks, about one week to about 5 weeks, about one week to about 4 weeks, about one week to about 3 weeks, about one week to about 2 weeks, about 2 weeks to about two months, about 2 weeks to about 7 weeks, about 2 weeks to about 6 weeks, about 2 weeks to about 5 weeks, about 2 weeks to about 4 weeks, about 2 weeks to about 3 weeks, about 3 weeks to about two months, about 3 weeks to about 7 weeks, about 3 weeks to about 6 weeks, about 3 weeks to about 5 weeks, about 3 weeks to about 4 weeks, about 4 weeks to about 2 months, about 4 weeks to about 7 weeks, about 4 weeks to about 6 weeks, about 4 weeks to about 5 weeks, about 5 weeks to about 2 months, about 5 weeks to about 7 weeks, about 5 weeks to about 6 weeks, about 6 weeks to about 2 months, about 6 weeks to about 7 weeks, or about 7 weeks to about 2 months). In some embodiments, the frequency between any two doses remains the same over a treatment period (period of time between the first administered dose and the last administered dose). In some embodiments, the induction doses are administered once a week. In some embodiments, the frequency between any two doses can be varied over a treatment period.

In some embodiments, the one or more maintenance doses are independently administered to the subject at about 100, 200, 400, 800, or 1600 mg of the antibody or antigen-binding protein. In some embodiments, the one or more maintenance dose is 800 mg of the antibody or antigen-binding fragment. In further embodiments, the one or more maintenance doses is 1600 mg of the antibody or antigen-binding fragment.

In certain embodiments, the one or more maintenance doses are independently administered to the subject at about 400 mg of the antibody or antigen-binding fragment to about 2,000 mg of the antibody or antigen-binding fragment (e.g., about 400 mg to about 1,950 mg, about 400 mg to about 1,900 mg, about 400 mg to about 1,850 mg, about 400 mg to about 1,800 mg, about 400 mg to about 1,750 mg, about 400 mg to about 1,700 mg, about 400 mg to about 1,650 mg, about 400 mg to about 1,600 mg, about 400 mg to about 1,550 mg, about 400 mg to about 1,500 mg, about 400 mg to about 1,450 mg, about 400 mg to about 1,400 mg, about 400 mg to about 1,350 mg, about 400 mg to about 1,300 mg, about 400 mg to about 1,250 mg, about 400 mg to about 1,200 mg, about 400 mg to about 1,150 mg, about 400 mg to about 1,100 mg, about 400 mg to about 1,050 mg, about 400 mg to about 1,000 mg, about 400 mg to about 950 mg, about 400 mg to about 900 mg, about 400 mg to about 900 mg, about 400 mg to about 850 mg, about 400 mg to about 800 mg, about 400 mg to about 750 mg, about 400 mg to about 700 mg, about 400 mg to about 650 mg, about 400 mg to about 600 mg, about 400 mg to about 550 mg, about 400 mg to about 500 mg, about 400 mg to about 450 mg, about 500 mg to about 2,000 mg, about 500 mg to about 1,950 mg, about 500 mg to about 1,900 mg, about 500 mg to about 1,850 mg, about 500 mg to about 1,800 mg, about 500 mg to about 1,750 mg, about 500 mg to about 1,700 mg, about 500 mg to about 1,650 mg, about 500 mg to about 1,600 mg, about 500 mg to about 1,550 mg, about 500 mg to about 1,500 mg, about 500 mg to about 1,450 mg, about 500 mg to about 1,400 mg, about 500 mg to about 1,350 mg, about 500 mg to about 1,300 mg, about 500 mg to about 1,250 mg, about 500 mg to about 1,200 mg, about 500 mg to about 1,150 mg, about 500 mg to about 1,100 mg, about 500 mg to about 1,050 mg, about 500 mg to about 1,000 mg, about 500 mg to about 950 mg, about 500 mg to about 900 mg, about 500 mg to about 900 mg, about 500 mg to about 850 mg, about 500 mg to about 800 mg, about 500 mg to about 750 mg, about 500 mg to about 700 mg, about 500 mg to about 650 mg, about 500 mg to about 600 mg, about 500 mg to about 550 mg, about 600 mg to about 2,000 mg, about 600 mg to about 1,950 mg, about 600 mg to about 1,900 mg, about 600 mg to about 1,850 mg, about 600 mg to about 1,800 mg, about 600 mg to about 1,750 mg, about 600 mg to about 1,700 mg, about 600 mg to about 1,650 mg, about 600 mg to about 1,600 mg, about 600 mg to about 1,550 mg, about 600 mg to about 1,500 mg, about 600 mg to about 1,450 mg, about 600 mg to about 1,400 mg, about 600 mg to about 1,350 mg, about 600 mg to about 1,300 mg, about 600 mg to about 1,250 mg, about 600 mg to about 1,200 mg, about 600 mg to about 1,150 mg, about 600 mg to about 1,100 mg, about 600 mg to about 1,050 mg, about 600 mg to about 1,000 mg, about 600 mg to about 950 mg, about 600 mg to about 900 mg, about 600 mg to about 900 mg, about 600 mg to about 850 mg, about 600 mg to about 800 mg, about 600 mg to about 750 mg, about 600 mg to about 700 mg, about 600 mg to about 650 mg, about 700 mg to about 2,000 mg, about 700 mg to about 1,950 mg, about 700 mg to about 1,900 mg, about 700 mg to about 1,850 mg, about 700 mg to about 1,800 mg, about 700 mg to about 1,750 mg, about 700 mg to about 1,700 mg, about 700 mg to about 1,650 mg, about 700 mg to about 1,600 mg, about 700 mg to about 1,550 mg, about 700 mg to about 1,500 mg, about 700 mg to about 1,450 mg, about 700 mg to about 1,400 mg, about 700 mg to about 1,350 mg, about 700 mg to about 1,300 mg, about 700 mg to about 1,250 mg, about 700 mg to about 1,200 mg, about 700 mg to about 1,150 mg, about 700 mg to about 1,100 mg, about 700 mg to about 1,050 mg, about 700 mg to about 1,000 mg, about 700 mg to about 950 mg, about 700 mg to about 900 mg, about 700 mg to about 900 mg, about 700 mg to about 850 mg, about 700 mg to about 800 mg, about 700 mg to about 750 mg, about 800 mg to about 2,000 mg, about 800 mg to about 1,950 mg, about 800 mg to about 1,900 mg, about 800 mg to about 1,850 mg, about 800 mg to about 1,800 mg, about 800 mg to about 1,750 mg, about 800 mg to about 1,700 mg, about 800 mg to about 1,650 mg, about 800 mg to about 1,600 mg, about 800 mg to about 1,550 mg, about 800 mg to about 1,500 mg, about 800 mg to about 1,450 mg, about 800 mg to about 1,400 mg, about 800 mg to about 1,350 mg, about 800 mg to about 1,300 mg, about 800 mg to about 1,250 mg, about 800 mg to about 1,200 mg, about 800 mg to about 1,150 mg, about 800 mg to about 1,100 mg, about 800 mg to about 1,050 mg, about 800 mg to about 1,000 mg, about 800 mg to about 950 mg, about 800 mg to about 900 mg, about 800 mg to about 900 mg, about 800 mg to about 850 mg, about 900 mg to about 2,000 mg, about 900 mg to about 1,950 mg, about 900 mg to about 1,900 mg, about 900 mg to about 1,850 mg, about 900 mg to about 1,800 mg, about 900 mg to about 1,750 mg, about 900 mg to about 1,700 mg, about 900 mg to about 1,650 mg, about 900 mg to about 1,600 mg, about 900 mg to about 1,550 mg, about 900 mg to about 1,500 mg, about 900 mg to about 1,450 mg, about 900 mg to about 1,400 mg, about 900 mg to about 1,350 mg, about 900 mg to about 1,300 mg, about 900 mg to about 1,250 mg, about 900 mg to about 1,200 mg, about 900 mg to about 1,150 mg, about 900 mg to about 1,100 mg, about 900 mg to about 1,050 mg, about 900 mg to about 1,000 mg, about 900 mg to about 950 mg, about 1,000 mg to about 2,000 mg, about 1,000 mg to about 1,950 mg, about 1,000 mg to about 1,900 mg, about 1,000 mg to about 1,850 mg, about 1,000 mg to about 1,800 mg, about 1,000 mg to about 1,750 mg, about 1,000 mg to about 1,700 mg, about 1,000 mg to about 1,650 mg, about 1,000 mg to about 1,600 mg, about 1,000 mg to about 1,550 mg, about 1,000 mg to about 1,500 mg, about 1,000 mg to about 1,450 mg, about 1,000 mg to about 1,400 mg, about 1,000 mg to about 1,350 mg, about 1,000 mg to about 1,300 mg, about 1,000 mg to about 1,250 mg, about 1,000 mg to about 1,200 mg, about 1,000 mg to about 1,150 mg, about 1,000 mg to about 1,100 mg, about 1,000 mg to about 1,050 mg, about 1,100 mg to about 2,000 mg, about 1,100 mg to about 1,950 mg, about 1,100 mg to about 1,900 mg, about 1,100 mg to about 1,850 mg, about 1,100 mg to about 1,800 mg, about 1,100 mg to about 1,750 mg, about 1,100 mg to about 1,700 mg, about 1,100 mg to about 1,650 mg, about 1,100 mg to about 1,600 mg, about 1,100 mg to about 1,550 mg, about 1,100 mg to about 1,500 mg, about 1,100 mg to about 1,450 mg, about 1,100 mg to about 1,400 mg, about 1,100 mg to about 1,350 mg, about 1,100 mg to about 1,300 mg, about 1,100 mg to about 1,250 mg, about 1,100 mg to about 1,200 mg, about 1,100 mg to about 1,150 mg, about 1,200 mg to about 2,000 mg, about 1,200 mg to about 1,950 mg, about 1,200 mg to about 1,900 mg, about 1,200 mg to about 1,850 mg, about 1,200 mg to about 1,800 mg, about 1,200 mg to about 1,750 mg, about 1,200 mg to about 1,700 mg, about 1,200 mg to about 1,650 mg, about 1,200 mg to about 1,600 mg, about 1,200 mg to about 1,550 mg, about 1,200 mg to about 1,500 mg, about 1,200 mg to about 1,450 mg, about 1,200 mg to about 1,400 mg, about 1,200 mg to about 1,350 mg, about 1,200 mg to about 1,300 mg, about 1,200 mg to about 1,250 mg, about 1,300 mg to about 2,000 mg, about 1,300 mg to about 1,950 mg, about 1,300 mg to about 1,900 mg, about 1,300 mg to about 1,850 mg, about 1,300 mg to about 1,800 mg, about 1,300 mg to about 1,750 mg, about 1,300 mg to about 1,700 mg, about 1,300 mg to about 1,650 mg, about 1,300 mg to about 1,600 mg, about 1,300 mg to about 1,550 mg, about 1,300 mg to about 1,500 mg, about 1,300 mg to about 1,450 mg, about 1,300 mg to about 1,400 mg, about 1,300 mg to about 1,350 mg, about 1,400 mg to about 2,000 mg, about 1,400 mg to about 1,950 mg, about 1,400 mg to about 1,900 mg, about 1,400 mg to about 1,850 mg, about 1,400 mg to about 1,800 mg, about 1,400 mg to about 1,750 mg, about 1,400 mg to about 1,700 mg, about 1,400 mg to about 1,650 mg, about 1,400 mg to about 1,600 mg, about 1,400 mg to about 1,550 mg, about 1,400 mg to about 1,500 mg, about 1,400 mg to about 1,450 mg, about 1,500 mg to about 2,000 mg, about 1,500 mg to about 1,950 mg, about 1,500 mg to about 1,900 mg, about 1,500 mg to about 1,850 mg, about 1,500 mg to about 1,800 mg, about 1,500 mg to about 1,750 mg, about 1,500 mg to about 1,700 mg, about 1,500 mg to about 1,650 mg, about 1,500 mg to about 1,600 mg, about 1,500 mg to about 1,550 mg, about 1,600 mg to about 2,000 mg, about 1,600 mg to about 1,950 mg, about 1,600 mg to about 1,900 mg, about 1,600 mg to about 1,850 mg, about 1,600 mg to about 1,800 mg, about 1,600 mg to about 1,750 mg, about 1,600 mg to about 1,700 mg, about 1,600 mg to about 1,650 mg, about 1,700 mg to about 2,000 mg, about 1,700 mg to about 1,950 mg, about 1,700 mg to about 1,900 mg, about 1,700 mg to about 1,850 mg, about 1,700 mg to about 1,800 mg, about 1,700 mg to about 1,750 mg, about 1,800 mg to about 2,000 mg, about 1,800 mg to about 1,950 mg, about 1,800 mg to about 1,900 mg, about 1,800 mg to about 1,850 mg, about 1,900 mg to about 2,000 mg, or about 1,900 mg to about 1,950 mg). In some embodiments, one or more 1600 mg maintenance doses of the pharmaceutical composition are independently administered to the subject.

In some embodiments, the subject is administered 1 to about 50 maintenance doses (e.g., 1 to about 45 doses, 1 to about 40 doses, 1 to about 35 doses, 1 to about 30 doses, 1 to about 25 doses, 1 to about 20 doses, 1 to about 18 doses, 1 to about 16 doses, 1 to about 14 doses, 1 to about 12 doses, 1 to about 10 doses, 1 to about 8 doses, 1 to about 6 doses, 1 to about 4 doses, 1 to about 2 doses, about 2 to about 50 doses, about 2 to about 45 doses, about 2 to about 40 doses, about 2 to about 35 doses, about 2 to about 30 doses, about 2 to about 25 doses, about 2 to about 20 doses, about 2 to about 18 doses, about 2 to about 16 doses, about 2 to about 14 doses, about 2 to about 12 doses, about 2 to about 10 doses, about 2 to about 8 doses, about 2 to about 6 doses, about 2 to about 4 doses, about 4 to about 50 doses, about 4 to about 45 doses, about 4 to about 40 doses, about 4 to about 35 doses, about 4 to about 30 doses, about 4 to about 25 doses, about 4 to about 20 doses, about 4 to about 18 doses, about 4 to about 16 doses, about 4 to about 14 doses, about 4 to about 12 doses, about 4 to about 10 doses, about 4 to about 8 doses, about 4 to about 6 doses, about 6 to about 50 doses, about 6 to about 45 doses, about 6 to about 40 doses, about 6 to about 35 doses, about 6 to about 30 doses, about 6 to about 25 doses, about 6 to about 20 doses, about 6 to about 18 doses, about 6 to about 16 doses, about 6 to about 14 doses, about 6 to about 12 doses, about 6 to about 10 doses, about 6 to about 8 doses, about 8 to about 50 doses, about 8 to about 45 doses, about 8 to about 40 doses, about 8 to about 35 doses, about 8 to about 30 doses, about 8 to about 25 doses, about 8 to about 20 doses, about 8 to about 18 doses, about 8 to about 16 doses, about 8 to about 14 doses, about 8 to about 12 doses, about 8 to about 10 doses, about 10 to about 50 doses, about 10 to about 45 doses, about 10 to about 40 doses, about 10 to about 35 doses, about 10 to about 30 doses, about 10 to about 25 doses, about 10 to about 20 doses, about 10 to about 18 doses, about 10 to about 16 doses, about 10 to about 14 doses, about 10 to about 12 doses, about 12 to about 50 doses, about 12 to about 45 doses, about 12 to about 40 doses, about 12 to about 35 doses, about 12 to about 30 doses, about 12 to about 25 doses, about 12 to about 20 doses, about 12 to about 18 doses, about 12 to about 16 doses, about 12 to about 14 doses, about 14 to about 50 doses, about 14 to about 45 doses, about 14 to about 40 doses, about 14 to about 35 doses, about 14 to about 30 doses, about 14 to about 25 doses, about 14 to about 20 doses, about 14 to about 18 doses, about 14 to about 16 doses, about 16 to about 50 doses, about 16 to about 45 doses, about 16 to about 40 doses, about 16 to about 35 doses, about 16 to about 30 doses, about 16 to about 25 doses, about 16 to about 20 doses, about 16 to about 18 doses, about 18 to about 50 doses, about 18 to about 45 doses, about 18 to about 40 doses, about 18 to about 35 doses, about 18 to about 30 doses, about 18 to about 25 doses, about 19 to about 20 doses, about 20 to about 50 doses, about 20 to about 45 doses, about 20 to about 40 doses, about 20 to about 35 doses, about 20 to about 30 doses, about 20 to about 25 doses, about 25 to about 50 doses, about 25 to about 45 doses, about 25 to about 40 doses, about 25 to about 35 doses, about 25 to about 30 doses, about 30 to about 50 doses, about 30 to about 45 doses, about 30 to about 40 doses, about 30 to about 35 doses, about 35 to about 50 doses, about 35 to about 45 doses, about 35 to about 40 doses, about 40 to about 50 doses, about 40 to about 45 doses, or about 45 to about 50 doses) of the pharmaceutical composition (e.g., any of the pharmaceutical compositions comprising any of the antibodies or antigen-binding fragments described herein).

In some embodiments, where two or more maintenance doses of the pharmaceutical composition (e.g., any of the pharmaceutical compositions comprising any of the antibodies or antigen-binding fragments described herein) are administered to the subject (e.g., any of the subjects described herein), any two consecutive maintenance doses can be administered at a frequency of about one week to about two months (e.g., about one week to about 7 weeks, about one week to about 6 weeks, about one week to about 5 weeks, about one week to about 4 weeks, about one week to about 3 weeks, about one week to about 2 weeks, about 2 weeks to about two months, about 2 weeks to about 7 weeks, about 2 weeks to about 6 weeks, about 2 weeks to about 5 weeks, about 2 weeks to about 4 weeks, about 2 weeks to about 3 weeks, about 3 weeks to about two months, about 3 weeks to about 7 weeks, about 3 weeks to about 6 weeks, about 3 weeks to about 5 weeks, about 3 weeks to about 4 weeks, about 4 weeks to about 2 months, about 4 weeks to about 7 weeks, about 4 weeks to about 6 weeks, about 4 weeks to about 5 weeks, about 5 weeks to about 2 months, about 5 weeks to about 7 weeks, about 5 weeks to about 6 weeks, about 6 weeks to about 2 months, about 6 weeks to about 7 weeks, or about 7 weeks to about 2 months). In some embodiments, the frequency between any two doses remains the same over a treatment period (period of time between the first administered dose and the last administered dose). In some embodiments, the maintenance doses are administered every two weeks. In some embodiments, the frequency between any two doses can be varied over a treatment period.

In some embodiments, the anti-BCMA antibody or antigen-binding fragment thereof as described herein are administered to the subject once every two weeks. For example, at least or about 800 mg of the anti-BCMA antibody or antigen-binding fragment thereof can be administered to the subject once every two weeks. In some cases, at least or about 1600 mg of the anti-BCMA antibody or antigen-binding fragment thereof can be administered to the subject once every two weeks.

In some embodiments, the anti-BCMA antibody or antigen-binding fragment thereof as described herein are administered to the subject once every week for the first 8 weeks, and then once every two weeks. For example, at least or about 800 mg of the anti-BCMA antibody or antigen-binding fragment thereof can be administered to the subject once every week for the first 8 weeks, and then once every two weeks. In some cases, at least or about 1600 mg of the anti-BCMA antibody or antigen-binding fragment thereof can be administered to the subject once every week for the first 8 weeks, and then once every two weeks.

In some embodiments, the anti-BCMA antibody or antigen-binding fragment thereof as described herein are administered to the subject once every week for two 28-day cycles, and then once every two weeks in the following 28-day cycles. For example, at least or about 800 mg of the anti-BCMA antibody or antigen-binding fragment thereof can be administered to the subject once every week for two 28-day cycles, and then once every two weeks in the following 28-day cycles. In some cases, at least or about 1600 mg of the anti-BCMA antibody or antigen-binding fragment thereof can be administered to the subject once every week for two 28-day cycles, and then once every two weeks in the following 28-day cycles.

C. Treatment Period

In some embodiments, the treatment period can be about 1 week to about 5 years (e.g., about 1 week to about 4.5 years, about 1 week to about 4 years, about 1 week to about 3.5 years, about 1 week to about 3 years, about 1 week to about 2.5 years, about 1 week to about 2 years, about 1 week to about 1.5 years, about 1 week to about 1 year, about 1 week to about 10 months, about 1 week to about 8 months, about 1 week to about 6 months, about 1 week to about 4 months, about 1 week to about 2 months, about 1 week to about 1 month, about 1 week to about 2 weeks, about 2 weeks to about 5 years, about 2 weeks to about 4.5 years, about 2 weeks to about 4 years, about 2 weeks to about 3.5 years, about 2 weeks to about 3 years, about 2 weeks to about 2.5 years, about 2 weeks to about 2 years, about 2 weeks to about 1.5 years, about 2 weeks to about 1 year, about 2 weeks to about 10 months, about 2 weeks to about 8 months, about 2 weeks to about 6 months, about 2 weeks to about 4 months, about 2 weeks to about 2 months, about 2 weeks to about 1 month, about 1 month to about 5 years, about 1 month to about 4.5 years, about 1 month to about 4 years, about 1 month to about 3.5 years, about 1 month to about 3 years, about 1 month to about 2.5 years, about 1 month to about 2 years, about 1 month to about 1.5 years, about 1 month to about 1 year, about 1 month to about 10 months, about 1 month to about 8 months, about 1 month to about 6 months, about 1 month to about 4 months, about 1 month to about 2 months, about 2 months to about 5 years, about 2 months to about 4.5 years, about 2 months to about 4 years, about 2 months to about 3.5 years, about 2 months to about 3 years, about 2 months to about 2.5 years, about 2 months to about 2 years, about 2 months to about 1.5 years, about 2 months to about 1 year, about 2 months to about 10 months, about 2 months to about 8 months, about 2 months to about 6 months, about 2 months to about 4 months, about 4 months to about 5 years, about 4 months to about 4.5 years, about 4 months to about 4 years, about 4 months to about 3.5 years, about 4 months to about 3 years, about 4 months to about 2.5 years, about 4 months to about 2 years, about 4 months to about 1.5 years, about 4 months to about 1 year, about 4 months to about 10 months, about 4 months to about 8 months, about 4 months to about 6 months, about 6 months to about 5 years, about 6 months to about 4.5 years, about 6 months to about 4 years, about 6 months to about 3.5 years, about 6 months to about 3 years, about 6 months to about 2.5 years, about 6 months to about 2 years, about 6 months to about 1.5 years, about 6 months to about 1 year, about 6 months to about 10 months, about 6 months to about 8 months, about 8 months to about 5 years, about 8 months to about 4.5 years, about 8 months to about 4 years, about 8 months to about 3.5 years, about 8 months to about 3 years, about 8 months to about 2.5 years, about 8 months to about 2 years, about 8 months to about 1.5 years, about 8 months to about 1 year, about 8 months to about 10 months, about 10 months to about 5 years, about 10 months to about 4.5 years, about 10 months to about 4 years, about 10 months to about 3.5 years, about 10 months to about 3 years, about 10 months to about 2.5 years, about 10 months to about 2 years, about 10 months to about 1.5 years, about 10 months to about 1 year, about 1 year to about 5 years, about 1 year to about 4.5 years, about 1 year to about 4 years, about 1 year to about 3.5 years, about 1 year to about 3 years, about 1 year to about 2.5 years, about 1 year to about 2 years, about 1 year to about 1.5 years, about 1.5 years to about 5 years, about 1.5 years to about 4.5 years, about 1.5 years to about 4 years, about 1.5 years to about 3.5 years, about 1.5 years to about 3 years, about 1.5 years to about 2.5 years, about 1.5 years to about 2 years, about 2 years to about 5 years, about 2 years to about 4.5 years, about 2 years to about 4 years, about 2 years to about 3.5 years, about 2 years to about 3 years, about 2 years to about 2.5 years, about 2.5 years to about 5 years, about 2.5 years to about 4.5 years, about 2.5 years to about 4 years, about 2/5 years to about 3.5 years, about 2.5 years to about 3 years, about 3 years to about 5 years, about 3 years to about 4.5 years, about 3 years to about 4 years, about 3 years to about 3.5 years, about 3.5 years to about 5 years, about 3.5 years to about 4.5 years, about 3.5 years to about 4 years, about 4 years to about 5 years, about 4 years to about 4.5 years, or about 4.5 years to about 5 years).

An effective treatment of multiple myeloma in a subject means one or more of a reduction in the severity of the disease, a decrease in the rate of development, and/or a reduction in one or more of the number, frequency, severity, and/or duration of one or more symptoms of multiple myeloma in a subject. In some instances, therapeutic efficacy can be observed in a subject relative to historical controls or past experience in the same subject. In other instances, therapeutic efficacy can be demonstrated in a preclinical or clinical trial in a population of treated subjects relative to a control population of untreated or placebo-treated subjects.

In some embodiments, a pharmaceutical composition (e.g., any of the exemplary pharmaceutical compositions described herein comprising any of the antibodies or antigen-binding fragments described herein) is administered at a frequency of once every two weeks. In some embodiments, a pharmaceutical composition (e.g., any of the exemplary pharmaceutical compositions described herein comprising any of the antibodies or antigen-binding fragments described herein) is administered at a 1600 mg fixed dose once a week. In some embodiments, a pharmaceutical composition (e.g., any of the exemplary pharmaceutical compositions described herein comprising any of the antibodies or antigen-binding fragments described herein) is administered at a 1600 mg fixed dose once every two weeks. In some embodiments, a pharmaceutical composition (e.g., any of the exemplary pharmaceutical compositions described herein comprising any of the antibodies or antigen-binding fragments described herein) is administered at an 800 mg fixed dose once a week. In some embodiments, a pharmaceutical composition (e.g., any of the exemplary pharmaceutical compositions described herein comprising any of the antibodies or antigen-binding fragments described herein) is administered at an 800 mg fixed dose once every two weeks.

In some embodiments, provided herein are methods of treating a subject having multiple myeloma, the method including administering to the subject one or more doses of a pharmaceutical composition comprising: (i) an antibody, or antigen-binding fragment thereof, that specifically binds to a B cell maturation antigen (BCMA), and (ii) a pharmaceutically acceptable carrier. In some embodiments, the multiple myeloma is relapsed or refractory multiple myeloma (RRMM). In some embodiments, the antibody, or antigen-binding fragment thereof comprises: a heavy chain variable region comprising a CDR1 comprising SEQ ID NO: 1, a CDR2 comprising SEQ ID NO: 2, and a CDR3 comprising SEQ ID NO: 3, and a light chain variable domain comprising a CDR1 comprising SEQ ID NO: 5, a CDR2 comprising SEQ ID NO: 6, and a CDR3 comprising SEQ ID NO: 7. In some embodiments, the antibody is an IgG1 antibody.

In some embodiments, one or more doses of 1600 mg of the antibody, or antigen-binding fragment thereof, is independently administered to the subject at a frequency of every two weeks. In some embodiments, one or more doses of 800 mg of the antibody, or antigen-binding fragment thereof, is independently administered to the subject at a frequency of every week. In some embodiments, about 1-2 induction doses of 1600 mg of the antibody, or antigen-binding fragment thereof, is independently administered to the subject at a frequency of every week, followed by one or more maintenance doses of 1600 mg of the antibody, or antigen-binding fragment thereof, independently administered to the subject at a frequency of every two weeks. In some embodiments, about 1-2 induction doses of 800 mg of the antibody, or antigen-binding fragment thereof, is independently administered to the subject at a frequency of every week, followed by one or more maintenance doses of 1600 mg of the antibody, or antigen-binding fragment thereof, independently administered to the subject at a frequency of every two weeks.

In some embodiments, the subject was previously administered one or more therapeutic agents or treatments for multiple myeloma. The one or more previously administered therapeutic agents or treatments for multiple myeloma include, but are not limited to, a proteasome inhibitor (PI), an immunomodulatory drug (IMiD), and an anti-CD38 antibody.

Specifically, proteasome inhibitors are agents whose mechanism of action is to inhibit a proteasome. Exemplary proteasome inhibitors include, but are not limited to are bortezomib, carfilzomib, and ixazomib. Immunomodulatory drugs (IMiDs) are thalidomide analogues, which possess pleiotropic anti-myeloma properties including immune-modulation, anti-angiogenic, anti-inflammatory and anti-proliferative effects. Immunomodulatory imide drugs (IMiDs) are immunomodulatory agents containing and “imide” group. Exemplary IMiDs include, but are not limited to, lenalidomide, pomalidomide, thalidomide, and Iberdomide (CC-220, Celgene). Exemplary anti-CD38 antibodies include, but are not limited to, daratumumab and isatuximab.

In some embodiments, the previously administered one or more therapeutic agents or treatments were not effective in treating the multiple myeloma. In some embodiments, the subject has one or more measurable diseases including a serum monoclonal paraprotein (M-protein) level of ≥0.5 g/dL, a urine M-protein level of ≥200 mg/24 hours, a serum immunoglobulin free light chain ≥10 mg/dL, and/or an abnormal serum immunoglobulin kappa to lambda free light chain ratio.

D. Routes of Administration

Administration of a pharmaceutical composition (e.g., any of the exemplary pharmaceutical compositions described herein comprising any of the antibodies or antigen-binding fragments described herein) can be parenteral. In some embodiments, administration of a pharmaceutical composition (e.g., any of the exemplary pharmaceutical compositions described herein comprising any of the antibodies or antigen-binding fragments described herein) can be intravenous, subcutaneous, intra-arterial, intracranial, intrathecal, intraperitoneal, or intramuscular. Administration can also be localized directly into a tumor. Administration into the systemic circulation by intravenous or subcutaneous administration. In some embodiments, the administration of a pharmaceutical composition (e.g., any of the exemplary pharmaceutical compositions described herein comprising any of the antibodies or antigen-binding fragments described herein) is systemic. In some embodiments, the systemic administration of a pharmaceutical composition (e.g., any of the exemplary pharmaceutical compositions described herein comprising any of the antibodies or antigen-binding fragments described herein) is intravenous administration.

Intravenous administration can be performed, for example, by step-wise infusion or a single bolus injection. In some embodiments, the step-wise infusion is performed using an infusion rate of about 20 mg/hour to about 500 mg/hour (e.g., about 20 mg/hour to about 450 mg/hour, about 20 mg/hour to about 400 mg/hour, about 20 mg/hour to about 350 mg/hour, about 20 mg/hour to about 300 mg/hour, about 20 mg/hour to about 250 mg/hour, about 20 mg/hour to about 200 mg/hour, about 20 mg/hour to about 180 mg/hour, about 20 mg/hour to about 160 mg/hour, about 20 mg/hour to about 140 mg/hour, about 20 mg/hour to about 120 mg/hour, about 20 mg/hour to about 100 mg/hour, about 20 mg/hour to about 80 mg/hour, about 20 mg/hour to about 60 mg/hour, about 20 mg/hour to about 50 mg/hour, about 20 mg/hour to about 40 mg/hour, about 40 mg/hour to about 500 mg/hour, about 40 mg/hour to about 450 mg/hour, about 40 mg/hour to about 400 mg/hour, about 40 mg/hour to about 350 mg/hour, about 40 mg/hour to about 300 mg/hour, about 40 mg/hour to about 250 mg/hour, about 40 mg/hour to about 200 mg/hour, about 40 mg/hour to about 180 mg/hour, about 40 mg/hour to about 160 mg/hour, about 40 mg/hour to about 140 mg/hour, about 40 mg/hour to about 120 mg/hour, about 40 mg/hour to about 100 mg/hour, about 40 mg/hour to about 80 mg/hour, about 40 mg/hour to about 60 mg/hour, about 40 mg/hour to about 50 mg/hour, about 50 mg/hour to about 500 mg/hour, about 50 mg/hour to about 450 mg/hour, about 50 mg/hour to about 400 mg/hour, about 50 mg/hour to about 350 mg/hour, about 50 mg/hour to about 300 mg/hour, about 50 mg/hour to about 250 mg/hour, about 50 mg/hour to about 200 mg/hour, about 50 mg/hour to about 180 mg/hour, about 50 mg/hour to about 160 mg/hour, about 50 mg/hour to about 140 mg/hour, about 50 mg/hour to about 120 mg/hour, about 50 mg/hour to about 100 mg/hour, about 50 mg/hour to about 80 mg/hour, about 50 mg/hour to about 60 mg/hour, about 60 mg/hour to about 500 mg/hour, about 60 mg/hour to about 450 mg/hour, about 60 mg/hour to about 400 mg/hour, about 60 mg/hour to about 350 mg/hour, about 60 mg/hour to about 300 mg/hour, about 60 mg/hour to about 250 mg/hour, about 60 mg/hour to about 200 mg/hour, about 60 mg/hour to about 180 mg/hour, about 60 mg/hour to about 160 mg/hour, about 60 mg/hour to about 140 mg/hour, about 60 mg/hour to about 120 mg/hour, about 60 mg/hour to about 100 mg/hour, about 60 mg/hour to about 80 mg/hour, about 80 mg/hour to about 500 mg/hour, about 80 mg/hour to about 450 mg/hour, about 80 mg/hour to about 400 mg/hour, about 80 mg/hour to about 350 mg/hour, about 80 mg/hour to about 300 mg/hour, about 80 mg/hour to about 250 mg/hour, about 80 mg/hour to about 200 mg/hour, about 80 mg/hour to about 180 mg/hour, about 80 mg/hour to about 160 mg/hour, about 80 mg/hour to about 140 mg/hour, about 80 mg/hour to about 120 mg/hour, about 80 mg/hour to about 100 mg/hour, about 100 mg/hour to about 500 mg/hour, about 100 mg/hour to about 450 mg/hour, about 100 mg/hour to about 400 mg/hour, about 100 mg/hour to about 350 mg/hour, about 100 mg/hour to about 300 mg/hour, about 100 mg/hour to about 250 mg/hour, about 100 mg/hour to about 200 mg/hour, about 100 mg/hour to about 180 mg/hour, about 100 mg/hour to about 160 mg/hour, about 100 mg/hour to about 140 mg/hour, about 100 mg/hour to about 120 mg/hour, about 120 mg/hour to about 500 mg/hour, about 120 mg/hour to about 450 mg/hour, about 120 mg/hour to about 400 mg/hour, about 120 mg/hour to about 350 mg/hour, about 120 mg/hour to about 300 mg/hour, about 120 mg/hour to about 250 mg/hour, about 120 mg/hour to about 200 mg/hour, about 120 mg/hour to about 180 mg/hour, about 120 mg/hour to about 160 mg/hour, about 120 mg/hour to about 140 mg/hour, about 140 mg/hour to about 500 mg/hour, about 140 mg/hour to about 450 mg/hour, about 140 mg/hour to about 400 mg/hour, about 140 mg/hour to about 350 mg/hour, about 140 mg/hour to about 300 mg/hour, about 140 mg/hour to about 250 mg/hour, about 140 mg/hour to about 200 mg/hour, about 140 mg/hour to about 180 mg/hour, about 140 mg/hour to about 160 mg/hour, about 160 mg/hour to about 500 mg/hour, about 160 mg/hour to about 450 mg/hour, about 160 mg/hour to about 400 mg/hour, about 160 mg/hour to about 350 mg/hour, about 160 mg/hour to about 300 mg/hour, about 160 mg/hour to about 250 mg/hour, about 160 mg/hour to about 200 mg/hour, about 160 mg/hour to about 180 mg/hour, about 180 mg/hour to about 500 mg/hour, about 180 mg/hour to about 450 mg/hour, about 180 mg/hour to about 400 mg/hour, about 180 mg/hour to about 350 mg/hour, about 180 mg/hour to about 300 mg/hour, about 180 mg/hour to about 250 mg/hour, about 180 mg/hour to about 200 mg/hour, about 200 mg/hour to about 500 mg/hour, about 200 mg/hour to about 450 mg/hour, about 200 mg/hour to about 400 mg/hour, about 200 mg/hour to about 350 mg/hour, about 200 mg/hour to about 300 mg/hour, about 200 mg/hour to about 250 mg/hour, about 250 mg/hour to about 500 mg/hour, about 250 mg/hour to about 450 mg/hour, about 250 mg/hour to about 400 mg/hour, about 250 mg/hour to about 350 mg/hour, about 250 mg/hour to about 300 mg/hour, about 300 mg/hour to about 500 mg/hour, about 300 mg/hour to about 450 mg/hour, about 300 mg/hour to about 400 mg/hour, about 300 mg/hour to about 350 mg/hour, about 350 mg/hour to about 500 mg/hour, about 350 mg/hour to about 450 mg/hour, about 350 mg/hour to about 400 mg/hour, about 400 mg/hour to about 500 mg/hour, about 400 mg/hour to about 450 mg/hour, or about 450 mg/hour to about 500 mg/hour).

In some embodiments, the step-wise infusion rate is increased about every 10 minutes. In some embodiments, the step-wise infusion rate is increased about every 20 minutes. In some embodiments, the step-wise infusion rate is increased about every 30 minutes. In some embodiments, the step-wise infusion rate is increased about every 40 minutes. In some embodiments, the step-wise infusion rate is increased about every 50 minutes. In some embodiments, the step-wise infusion rate is increased about every 60 minutes. In some embodiments, during the step-wise infusion, the infusion rate is increased no more than about two-fold, about every 30 minute.

E. Pharmacokinetic Effects

In some embodiments, the administration of the pharmaceutical composition described herein, using any of the methods described herein, results in a steady-state concentration of the antibody or antigen-binding fragment thereof, in the serum of the subject that is able to bind to at least 50%, at least 60%, at least 70%, at least 80%, at least 90%, at least 91%, at least 92%, at least 93%, at least 94%, at least 95%, at least 96%, at least 97%, at least 98%, or at least 99% of the BCMA expressed on the surface of tumor cells in the subject.

In certain embodiments, the antibody or antigen-binding fragment is administered under dose and infusion rates such that the half-life of the antibody or antigen-binding fragment is at least 4, 5, 6, 7, 8, 9, 10, 11, 12, 13, 14 or 15 days. In other embodiments, the half-life is at least one week, at least two weeks, at least three weeks, or at least four weeks.

Some embodiments of these methods result in a steady-state concentration of the antibody, or antigen-binding fragment thereof, in the serum of the subject of about 1 μg/mL to about 200 μg/mL (e.g., about 1 μg/mL to about 180 μg/mL, about 1 μg/mL to about 160 μg/mL, about 1 μg/mL to about 140 μg/mL, about 1 μg/mL to about 120 μg/mL, about 1 μg/mL to about 100 μg/mL, about 1 μg/mL to about 90 μg/mL, about 1 μg/mL to about 80 μg/mL, about 1 μg/mL to about 70 μg/mL, about 1 μg/mL to about 60 μg/mL, about 1 μg/mL to about 50 μg/mL, about 1 μg/mL to about 40 μg/mL, about 1 μg/mL to about 30 μg/mL, about 1 μg/mL to about 20 μg/mL, about 1 μg/mL to about 10 μg/mL, about 10 μg/mL to about 200 μg/mL, about 10 μg/mL to about 180 μg/mL, about 10 μg/mL to about 160 μg/mL, about 10 μg/mL to about 140 μg/mL, about 10 μg/mL to about 120 μg/mL, about 10 μg/mL to about 100 μg/mL, about 10 μg/mL to about 90 μg/mL, about 10 μg/mL to about 80 μg/mL, about 10 μg/mL to about 70 μg/mL, about 10 μg/mL to about 60 μg/mL, about 10 μg/mL to about 50 μg/mL, about 10 μg/mL to about 40 μg/mL, about 10 μg/mL to about 30 μg/mL, about 10 μg/mL to about 20 μg/mL, about 20 μg/mL to about 200 μg/mL, about 20 μg/mL to about 180 μg/mL, about 20 μg/mL to about 160 μg/mL, about 20 μg/mL to about 140 μg/mL, about 20 μg/mL to about 120 μg/mL, about 20 μg/mL to about 100 μg/mL, about 20 μg/mL to about 90 μg/mL, about 20 μg/mL to about 80 μg/mL, about 20 μg/mL to about 70 μg/mL, about 20 μg/mL to about 60 μg/mL, about 20 μg/mL to about 50 μg/mL, about 20 μg/mL to about 40 μg/mL, about 20 μg/mL to about 30 μg/mL, about 30 μg/mL to about 200 μg/mL, about 30 μg/mL to about 180 μg/mL, about 30 μg/mL to about 160 μg/mL, about 30 μg/mL to about 140 μg/mL, about 30 μg/mL to about 120 μg/mL, about 30 μg/mL to about 100 μg/mL, about 30 μg/mL to about 90 μg/mL, about 30 μg/mL to about 80 μg/mL, about 30 μg/mL to about 70 μg/mL, about 30 μg/mL to about 60 μg/mL, about 30 μg/mL to about 50 μg/mL, about 30 μg/mL to about 40 μg/mL, about 40 μg/mL to about 200 μg/mL, about 40 μg/mL to about 180 μg/mL, about 40 μg/mL to about 160 μg/mL, about 40 μg/mL to about 140 μg/mL, about 40 μg/mL to about 120 μg/mL, about 40 μg/mL to about 100 μg/mL, about 40 μg/mL to about 90 μg/mL, about 40 μg/mL to about 80 μg/mL, about 40 μg/mL to about 70 μg/mL, about 40 μg/mL to about 60 μg/mL, about 40 μg/mL to about 50 μg/mL, about 50 μg/mL to about 200 μg/mL, about 50 μg/mL to about 180 μg/mL, about 50 μg/mL to about 160 μg/mL, about 50 μg/mL to about 140 μg/mL, about 50 μg/mL to about 120 μg/mL, about 50 μg/mL to about 100 μg/mL, about 50 μg/mL to about 90 μg/mL, about 50 μg/mL to about 80 μg/mL, about 50 μg/mL to about 70 μg/mL, about 50 μg/mL to about 60 μg/mL, about 60 μg/mL to about 200 μg/mL, about 60 μg/mL to about 180 μg/mL, about 60 μg/mL to about 160 μg/mL, about 60 μg/mL to about 140 μg/mL, about 60 μg/mL to about 120 μg/mL, about 60 μg/mL to about 100 μg/mL, about 60 μg/mL to about 90 μg/mL, about 60 μg/mL to about 80 μg/mL, about 60 μg/mL to about 70 μg/mL, about 70 μg/mL to about 200 μg/mL, about 70 μg/mL to about 180 μg/mL, about 70 μg/mL to about 160 μg/mL, about 70 μg/mL to about 140 μg/mL, about 70 μg/mL to about 120 μg/mL, about 70 μg/mL to about 100 μg/mL, about 70 μg/mL to about 90 μg/mL, about 70 μg/mL to about 80 μg/mL, about 80 μg/mL to about 200 μg/mL, about 80 μg/mL to about 180 μg/mL, about 80 μg/mL to about 160 μg/mL, about 80 μg/mL to about 140 μg/mL, about 80 μg/mL to about 120 μg/mL, about 80 μg/mL to about 100 μg/mL, about 80 μg/mL to about 90 μg/mL, about 90 μg/mL to about 200 μg/mL, about 90 μg/mL to about 180 μg/mL, about 90 μg/mL to about 160 μg/mL, about 90 μg/mL to about 140 μg/mL, about 90 μg/mL to about 120 μg/mL, about 90 μg/mL to about 100 μg/mL, about 100 μg/mL to about 200 μg/mL, about 100 μg/mL to about 180 μg/mL, about 100 μg/mL to about 160 μg/mL, about 100 μg/mL to about 140 μg/mL, about 100 μg/mL to about 120 μg/mL, about 120 μg/mL to about 200 μg/mL, about 120 μg/mL to about 180 μg/mL, about 120 μg/mL to about 160 μg/mL, about 120 μg/mL to about 140 μg/mL, about 140 μg/mL to about 200 μg/mL, about 140 μg/mL to about 180 μg/mL, about 140 μg/mL to about 160 μg/mL, about 160 μg/mL to about 200 μg/mL, about 160 μg/mL to about 180 μg/mL, or about 180 μg/mL to about 200 μg/mL) (e.g., for about 6 hours to about one year (e.g., about 6 hours to about 11.5 months, about 6 hours to about 11.0 months, about 6 hours to about 10.5 months, about 6 hours to about 10.0 months, about 6 hours to about 9.5 months, about 6 hours to about 9.0 months, about 6 hours to about 8.5 months, about 6 hours to about 8.0 months, about 6 hours to about 7.5 months, about 6 hours to about 7.0 months, about 6 hours to about 6.5 months, about 6 hours to about 6.0 months, about 6 hours to about 5.5 months, about 6 hours to about 5.0 months, about 6 hours to about 4.5 months, about 6 hours to about 4.0 months, about 6 hours to about 3.5 months, about 6 hours to about 3.0 months, about 6 hours to about 2.5 months, about 6 hours to about 2.0 months, about 6 hours to about 1.5 months, about 6 hours to about 5 weeks, about 6 hours to about 4 weeks, about 6 hours to about 3 weeks, about 6 hours to about 2 weeks, about 6 hours to about 1 week, about 6 hours to about 5 days, about 6 hours to about 3 days, about 6 hours to about 1 day, about 6 hours to about 18 hours, about 6 hours to about 12 hours, about 12 hours to about 1 year, about 12 hours to about 11.5 months, about 12 hours to about 11.0 months, about 12 hours to about 10.5 months, about 12 hours to about 10.0 months, about 12 hours to about 9.5 months, about 12 hours to about 9.0 months, about 12 hours to about 8.5 months, about 12 hours to about 8.0 months, about 12 hours to about 7.5 months, about 12 hours to about 7.0 months, about 12 hours to about 6.5 months, about 12 hours to about 6.0 months, about 12 hours to about 5.5 months, about 12 hours to about 5.0 months, about 12 hours to about 4.5 months, about 12 hours to about 4.0 months, about 12 hours to about 3.5 months, about 12 hours to about 3.0 months, about 12 hours to about 2.5 months, about 12 hours to about 2.0 months, about 12 hours to about 1.5 months, about 12 hours to about 5 weeks, about 12 hours to about 4 weeks, about 12 hours to about 3 weeks, about 12 hours to about 2 weeks, about 12 hours to about 1 week, about 12 hours to about 5 days, about 12 hours to about 3 days, about 12 hours to about 1 day, about 12 hours to about 18 hours, about 18 hours to about 1 year, about 18 hours to about 11.5 months, about 18 hours to about 11.0 months, about 18 hours to about 10.5 months, about 18 hours to about 10.0 months, about 18 hours to about 9.5 months, about 18 hours to about 9.0 months, about 18 hours to about 8.5 months, about 18 hours to about 8.0 months, about 18 hours to about 7.5 months, about 18 hours to about 7.0 months, about 18 hours to about 6.5 months, about 18 hours to about 6.0 months, about 18 hours to about 5.5 months, about 18 hours to about 5.0 months, about 18 hours to about 4.5 months, about 18 hours to about 4.0 months, about 18 hours to about 3.5 months, about 18 hours to about 3.0 months, about 18 hours to about 2.5 months, about 18 hours to about 2.0 months, about 18 hours to about 1.5 months, about 18 hours to about 5 weeks, about 18 hours to about 4 weeks, about 18 hours to about 3 weeks, about 18 hours to about 2 weeks, about 18 hours to about 1 week, about 18 hours to about 5 days, about 18 hours to about 3 days, about 18 hours to about 1 day, about 1 day to about 1 year, about 1 day to about 11.5 months, about 1 day to about 11.0 months, about 1 day to about 10.5 months, about 1 day to about 10.0 months, about 1 day to about 9.5 months, about 1 day to about 9.0 months, about 1 day to about 8.5 months, about 1 day to about 8.0 months, about 1 day to about 7.5 months, about 1 day to about 7.0 months, about 1 day to about 6.5 months, about 1 day to about 6.0 months, about 1 day to about 5.5 months, about 1 day to about 5.0 months, about 1 day to about 4.5 months, about 1 day to about 4.0 months, about 1 day to about 3.5 months, about 1 day to about 3.0 months, about 1 day to about 2.5 months, about 1 day to about 2.0 months, about 1 day to about 1.5 months, about 1 day to about 5 weeks, about 1 day to about 4 weeks, about 1 day to about 3 weeks, about 1 day to about 2 weeks, about 1 day to about 1 week, about 1 day to about 5 days, about 1 day to about 3 days, about 3 days to about 1 year, about 3 days to about 11.5 months, about 3 days to about 11.0 months, about 3 days to about 10.5 months, about 3 days to about 10.0 months, about 3 days to about 9.5 months, about 3 days to about 9.0 months, about 3 days to about 8.5 months, about 3 days to about 8.0 months, about 3 days to about 7.5 months, about 3 days to about 7.0 months, about 3 days to about 6.5 months, about 3 days to about 6.0 months, about 3 days to about 5.5 months, about 3 days to about 5.0 months, about 3 days to about 4.5 months, about 3 days to about 4.0 months, about 3 days to about 3.5 months, about 3 days to about 3.0 months, about 3 days to about 2.5 months, about 3 days to about 2.0 months, about 3 days to about 1.5 months, about 3 days to about 5 weeks, about 3 days to about 4 weeks, about 3 days to about 3 weeks, about 3 days to about 2 weeks, about 3 days to about 1 week, about 3 days to about 5 days, about 5 days to about 1 year, about 5 days to about 11.5 months, about 5 days to about 11.0 months, about 5 days to about 10.5 months, about 5 days to about 10.0 months, about 5 days to about 9.5 months, about 5 days to about 9.0 months, about 5 days to about 8.5 months, about 5 days to about 8.0 months, about 5 days to about 7.5 months, about 5 days to about 7.0 months, about 5 days to about 6.5 months, about 5 days to about 6.0 months, about 5 days to about 5.5 months, about 5 days to about 5.0 months, about 5 days to about 4.5 months, about 5 days to about 4.0 months, about 5 days to about 3.5 months, about 5 days to about 3.0 months, about 5 days to about 2.5 months, about 5 days to about 2.0 months, about 5 days to about 1.5 months, about 5 days to about 5 weeks, about 5 days to about 4 weeks, about 5 days to about 3 weeks, about 5 days to about 2 weeks, about 5 days to about 1 week, about 1 week to about 1 year, about 1 week to about 11.5 months, about 1 week to about 11.0 months, about 1 week to about 10.5 months, about 1 week to about 10.0 months, about 1 week to about 9.5 months, about 1 week to about 9.0 months, about 1 week to about 8.5 months, about 1 week to about 8.0 months, about 1 week to about 7.5 months, about 1 week to about 7.0 months, about 1 week to about 6.5 months, about 1 week to about 6.0 months, about 1 week to about 5.5 months, about 1 week to about 5.0 months, about 1 week to about 4.5 months, about 1 week to about 4.0 months, about 1 week to about 3.5 months, about 1 week to about 3.0 months, about 1 week to about 2.5 months, about 1 week to about 2.0 months, about 1 week to about 1.5 months, about 1 week to about 5 weeks, about 1 week to about 4 weeks, about 1 week to about 3 weeks, about 1 week to about 2 weeks, about 2 weeks to about 1 year, about 2 weeks to about 11.5 months, about 2 weeks to about 11.0 months, about 2 weeks to about 10.5 months, about 2 weeks to about 10.0 months, about 2 weeks to about 9.5 months, about 2 weeks to about 9.0 months, about 2 weeks to about 8.5 months, about 2 weeks to about 8.0 months, about 2 weeks to about 7.5 months, about 2 weeks to about 7.0 months, about 2 weeks to about 6.5 months, about 2 weeks to about 6.0 months, about 2 weeks to about 5.5 months, about 2 weeks to about 5.0 months, about 2 weeks to about 4.5 months, about 2 weeks to about 4.0 months, about 2 weeks to about 3.5 months, about 2 weeks to about 3.0 months, about 2 weeks to about 2.5 months, about 2 weeks to about 2.0 months, about 2 weeks to about 1.5 months, about 2 weeks to about 5 weeks, about 2 weeks to about 4 weeks, about 2 weeks to about 3 weeks, about 3 weeks to about 1 year, about 3 weeks to about 11.5 months, about 3 weeks to about 11.0 months, about 3 weeks to about 10.5 months, about 3 weeks to about 10.0 months, about 3 weeks to about 9.5 months, about 3 weeks to about 9.0 months, about 3 weeks to about 8.5 months, about 3 weeks to about 8.0 months, about 3 weeks to about 7.5 months, about 3 weeks to about 7.0 months, about 3 weeks to about 6.5 months, about 3 weeks to about 6.0 months, about 3 weeks to about 5.5 months, about 3 weeks to about 5.0 months, about 3 weeks to about 4.5 months, about 3 weeks to about 4.0 months, about 3 weeks to about 3.5 months, about 3 weeks to about 3.0 months, about 3 weeks to about 2.5 months, about 3 weeks to about 2.0 months, about 3 weeks to about 1.5 months, about 3 weeks to about 5 weeks, about 3 weeks to about 4 weeks, about 4 weeks to about 1 year, about 4 weeks to about 11.5 months, about 4 weeks to about 11.0 months, about 4 weeks to about 10.5 months, about 4 weeks to about 10.0 months, about 4 weeks to about 9.5 months, about 4 weeks to about 9.0 months, about 4 weeks to about 8.5 months, about 4 weeks to about 8.0 months, about 4 weeks to about 7.5 months, about 4 weeks to about 7.0 months, about 4 weeks to about 6.5 months, about 4 weeks to about 6.0 months, about 4 weeks to about 5.5 months, about 4 weeks to about 5.0 months, about 4 weeks to about 4.5 months, about 4 weeks to about 4.0 months, about 4 weeks to about 3.5 months, about 4 weeks to about 3.0 months, about 4 weeks to about 2.5 months, about 4 weeks to about 2.0 months, about 4 weeks to about 1.5 months, about 4 weeks to about 5 weeks, about 5 weeks to about 1 year, about 5 weeks to about 11.5 months, about 5 weeks to about 11.0 months, about 5 weeks to about 10.5 months, about 5 weeks to about 10.0 months, about 5 weeks to about 9.5 months, about 5 weeks to about 9.0 months, about 5 weeks to about 8.5 months, about 5 weeks to about 8.0 months, about 5 weeks to about 7.5 months, about 5 weeks to about 7.0 months, about 5 weeks to about 6.5 months, about 5 weeks to about 6.0 months, about 5 weeks to about 5.5 months, about 5 weeks to about 5.0 months, about 5 weeks to about 4.5 months, about 5 weeks to about 4.0 months, about 5 weeks to about 3.5 months, about 5 weeks to about 3.0 months, about 5 weeks to about 2.5 months, about 5 weeks to about 2.0 months, about 5 weeks to about 1.5 months, about 1.5 months to about 1 year, about 1.5 months to about 11.5 months, about 1.5 months to about 11.0 months, about 1.5 months to about 10.5 months, about 1.5 months to about 10.0 months, about 1.5 months to about 9.5 months, about 1.5 months to about 9.0 months, about 1.5 months to about 8.5 months, about 1.5 months to about 8.0 months, about 1.5 months to about 7.5 months, about 1.5 months to about 7.0 months, about 1.5 months to about 6.5 months, about 1.5 months to about 6.0 months, about 1.5 months to about 5.5 months, about 1.5 months to about 5.0 months, about 1.5 months to about 4.5 months, about 1.5 months to about 4.0 months, about 1.5 months to about 3.5 months, about 1.5 months to about 3.0 months, about 1.5 months to about 2.5 months, about 1.5 months to about 2.0 months, about 2.0 months to about 1 year, about 2.0 months to about 11.5 months, about 2.0 months to about 11.0 months, about 2.0 months to about 10.5 months, about 2.0 months to about 10.0 months, about 2.0 months to about 9.5 months, about 2.0 months to about 9.0 months, about 2.0 months to about 8.5 months, about 2.0 months to about 8.0 months, about 2.0 months to about 7.5 months, about 2.0 months to about 7.0 months, about 2.0 months to about 6.5 months, about 2.0 months to about 6.0 months, about 2.0 months to about 5.5 months, about 2.0 months to about 5.0 months, about 2.0 months to about 4.5 months, about 2.0 months to about 4.0 months, about 2.0 months to about 3.5 months, about 2.0 months to about 3.0 months, about 2.0 months to about 2.5 months, about 2.5 months to about 1 year, about 2.5 months to about 11.5 months, about 2.5 months to about 11.0 months, about 2.5 months to about 10.5 months, about 2.5 months to about 10.0 months, about 2.5 months to about 9.5 months, about 2.5 months to about 9.0 months, about 2.5 months to about 8.5 months, about 2.5 months to about 8.0 months, about 2.5 months to about 7.5 months, about 2.5 months to about 7.0 months, about 2.5 months to about 6.5 months, about 2.5 months to about 6.0 months, about 2.5 months to about 5.5 months, about 2.5 months to about 5.0 months, about 2.5 months to about 4.5 months, about 2.5 months to about 4.0 months, about 2.5 months to about 3.5 months, about 2.5 months to about 3.0 months, about 3.0 months to about 1 year, about 3.0 months to about 11.5 months, about 3.0 months to about 11.0 months, about 3.0 months to about 10.5 months, about 3.0 months to about 10.0 months, about 3.0 months to about 9.5 months, about 3.0 months to about 9.0 months, about 3.0 months to about 8.5 months, about 3.0 months to about 8.0 months, about 3.0 months to about 7.5 months, about 3.0 months to about 7.0 months, about 3.0 months to about 6.5 months, about 3.0 months to about 6.0 months, about 3.0 months to about 5.5 months, about 3.0 months to about 5.0 months, about 3.0 months to about 4.5 months, about 3.0 months to about 4.0 months, about 3.0 months to about 3.5 months, about 3.5 months to about 1 year, about 3.5 months to about 11.5 months, about 3.5 months to about 11.0 months, about 3.5 months to about 10.5 months, about 3.5 months to about 10.0 months, about 3.5 months to about 9.5 months, about 3.5 months to about 9.0 months, about 3.5 months to about 8.5 months, about 3.5 months to about 8.0 months, about 3.5 months to about 7.5 months, about 3.5 months to about 7.0 months, about 3.5 months to about 6.5 months, about 3.5 months to about 6.0 months, about 3.5 months to about 5.5 months, about 3.5 months to about 5.0 months, about 3.5 months to about 4.5 months, about 3.5 months to about 4.0 months, about 4.0 months to about 1 year, about 4.0 months to about 11.5 months, about 4.0 months to about 11.0 months, about 4.0 months to about 10.5 months, about 4.0 months to about 10.0 months, about 4.0 months to about 9.5 months, about 4.0 months to about 9.0 months, about 4.0 months to about 8.5 months, about 4.0 months to about 8.0 months, about 4.0 months to about 7.5 months, about 4.0 months to about 7.0 months, about 4.0 months to about 6.5 months, about 4.0 months to about 6.0 months, about 4.0 months to about 5.5 months, about 4.0 months to about 5.0 months, about 4.0 months to about 4.5 months, about 4.5 months to about 1 year, about 4.5 months to about 11.5 months, about 4.5 months to about 11.0 months, about 4.5 months to about 10.5 months, about 4.5 months to about 10.0 months, about 4.5 months to about 9.5 months, about 4.5 months to about 9.0 months, about 4.5 months to about 8.5 months, about 4.5 months to about 8.0 months, about 4.5 months to about 7.5 months, about 4.5 months to about 7.0 months, about 4.5 months to about 6.5 months, about 4.5 months to about 6.0 months, about 4.5 months to about 5.5 months, about 4.5 months to about 5.0 months, about 5.0 months to about 1 year, about 5.0 months to about 11.5 months, about 5.0 months to about 11.0 months, about 5.0 months to about 10.5 months, about 5.0 months to about 10.0 months, about 5.0 months to about 9.5 months, about 5.0 months to about 9.0 months, about 5.0 months to about 8.5 months, about 5.0 months to about 8.0 months, about 5.0 months to about 7.5 months, about 5.0 months to about 7.0 months, about 5.0 months to about 6.5 months, about 5.0 months to about 6.0 months, about 5.0 months to about 5.5 months, about 5.5 months to about 1 year, about 5.5 months to about 11.5 months, about 5.5 months to about 11.0 months, about 5.5 months to about 10.5 months, about 5.5 months to about 10.0 months, about 5.5 months to about 9.5 months, about 5.5 months to about 9.0 months, about 5.5 months to about 8.5 months, about 5.5 months to about 8.0 months, about 5.5 months to about 7.5 months, about 5.5 months to about 7.0 months, about 5.5 months to about 6.5 months, about 5.5 months to about 6.0 months, about 6.0 months to about 1 year, about 6.0 months to about 11.5 months, about 6.0 months to about 11.0 months, about 6.0 months to about 10.5 months, about 6.0 months to about 10.0 months, about 6.0 months to about 9.5 months, about 6.0 months to about 9.0 months, about 6.0 months to about 8.5 months, about 6.0 months to about 8.0 months, about 6.0 months to about 7.5 months, about 6.0 months to about 7.0 months, about 6.0 months to about 6.5 months, about 6.5 months to about 1 year, about 6.5 months to about 11.5 months, about 6.5 months to about 11.0 months, about 6.5 months to about 10.5 months, about 6.5 months to about 10.0 months, about 6.5 months to about 9.5 months, about 6.5 months to about 9.0 months, about 6.5 months to about 8.5 months, about 6.5 months to about 8.0 months, about 6.5 months to about 7.5 months, about 6.5 months to about 7.0 months, about 7.0 months to about 1 year, about 7.0 months to about 11.5 months, about 7.0 months to about 11.0 months, about 7.0 months to about 10.5 months, about 7.0 months to about 10.0 months, about 7.0 months to about 9.5 months, about 7.0 months to about 9.0 months, about 7.0 months to about 8.5 months, about 7.0 months to about 8.0 months, about 7.0 months to about 7.5 months, about 7.5 months to about 1 year, about 7.5 months to about 11.5 months, about 7.5 months to about 11.0 months, about 7.5 months to about 10.5 months, about 7.5 months to about 10.0 months, about 7.5 months to about 9.5 months, about 7.5 months to about 9.0 months, about 7.5 months to about 8.5 months, about 7.5 months to about 8.0 months, about 8.0 months to about 1 year, about 8.0 months to about 11.5 months, about 8.0 months to about 11.0 months, about 8.0 months to about 10.5 months, about 8.0 months to about 10.0 months, about 8.0 months to about 9.5 months, about 8.0 months to about 9.0 months, about 8.0 months to about 8.5 months, about 8.5 months to about 1 year, about 8.5 months to about 11.5 months, about 8.5 months to about 11.0 months, about 8.5 months to about 10.5 months, about 8.5 months to about 10.0 months, about 8.5 months to about 9.5 months, about 8.5 months to about 9.0 months, about 9.0 months to about 1 year, about 9.0 months to about 11.5 months, about 9.0 months to about 11.0 months, about 9.0 months to about 10.5 months, about 9.0 months to about 10.0 months, about 9.0 months to about 9.5 months, about 9.5 months to about 1 year, about 9.5 months to about 11.5 months, about 9.5 months to about 11.0 months, about 9.5 months to about 10.5 months, about 9.5 months to about 10.0 months, about 10.0 months to about 1 year, about 10.0 months to about 11.5 months, about 10.0 months to about 11.0 months, about 10.0 months to about 10.5 months, about 10.5 months to about 1 year, about 10.5 months to about 11.5 months, about 10.5 months to about 11.0 months, about 11.0 months to about 1 year, about 11.0 months to about 11.5 months, or about 11.5 months to about 1 year) after the administration of a first dose of the antibody or the antigen-binding fragment to the subject).

Some embodiments of these methods result in a steady-state concentration of free light chain (FLC), in the serum of the subject of less than about 50 mg/dL, less than about 45 mg/dL, less than about 40 mg/dL, less than about 35 mg/dL, less than about 30 mg/dL, less than about 25 mg/dL, less than about 20 mg/dL, less than about 18 mg/dL, less than about 16 mg/dL, less than about 14 mg/dL, less than about 12 mg/dL, less than about 10 mg/dL, less than about 8 mg/dL, less than about 6 mg/dL, less than about 4 mg/dL, less than about 2 mg/dL, or less than about 1 mg/dL (e.g., for about 6 hours to about one year, or any of the subranges of this range, after the administration of a first dose of the antibody or the antigen-binding fragment to the subject).

Some embodiments of these methods result in a steady-state concentration of free light chain (FLC), in the serum of the subject of about 0.1 mg/dL to about 50 mg/dL (e.g., about 0.1 mg/dL to about 48 mg/dL, about 0.1 mg/dL to about 45 mg/dL, about 0.1 mg/dL to about 40 mg/dL, about 0.1 mg/dL to about 35 mg/dL, about 0.1 mg/dL to about 30 mg/dL, about 0.1 mg/dL to about 25 mg/dL, about 0.1 mg/dL to about 20 mg/dL, about 0.1 mg/dL to about 18 mg/dL, about 0.1 mg/dL to about 16 mg/dL, about 0.1 mg/dL to about 14 mg/dL, about 0.1 mg/dL to about 12 mg/dL, about 0.1 mg/dL to about 10 mg/dL, about 0.1 mg/dL to about 8 mg/dL, about 0.1 mg/dL to about 6 mg/dL, about 0.1 mg/dL to about 4 mg/dL, about 0.1 mg/dL to about 2 mg/dL, about 0.1 mg/dL to about 1.0 mg/dL, about 0.1 mg/dL to about 0.5 mg/dL, about 0.1 mg/dL to about 0.2 mg/dL, about 0.2 mg/dL to about 50 mg/dL, about 0.2 mg/dL to about 48 mg/dL, about 0.2 mg/dL to about 45 mg/dL, about 0.2 mg/dL to about 40 mg/dL, about 0.2 mg/dL to about 35 mg/dL, about 0.2 mg/dL to about 30 mg/dL, about 0.2 mg/dL to about 25 mg/dL, about 0.2 mg/dL to about 20 mg/dL, about 0.2 mg/dL to about 18 mg/dL, about 0.2 mg/dL to about 16 mg/dL, about 0.2 mg/dL to about 14 mg/dL, about 0.2 mg/dL to about 12 mg/dL, about 0.2 mg/dL to about 10 mg/dL, about 0.2 mg/dL to about 8 mg/dL, about 0.2 mg/dL to about 6 mg/dL, about 0.2 mg/dL to about 4 mg/dL, about 0.2 mg/dL to about 2 mg/dL, about 0.2 mg/dL to about 1.0 mg/dL, about 0.2 mg/dL to about 0.5 mg/dL, about 0.5 mg/dL to about 50 mg/dL, about 0.5 mg/dL to about 48 mg/dL, about 0.5 mg/dL to about 45 mg/dL, about 0.5 mg/dL to about 40 mg/dL, about 0.5 mg/dL to about 35 mg/dL, about 0.5 mg/dL to about 30 mg/dL, about 0.5 mg/dL to about 25 mg/dL, about 0.5 mg/dL to about 20 mg/dL, about 0.5 mg/dL to about 18 mg/dL, about 0.5 mg/dL to about 16 mg/dL, about 0.5 mg/dL to about 14 mg/dL, about 0.5 mg/dL to about 12 mg/dL, about 0.5 mg/dL to about 10 mg/dL, about 0.5 mg/dL to about 8 mg/dL, about 0.5 mg/dL to about 6 mg/dL, about 0.5 mg/dL to about 4 mg/dL, about 0.5 mg/dL to about 2 mg/dL, about 0.5 mg/dL to about 1.0 mg/dL, about 1.0 mg/dL to about 50 mg/dL, about 1.0 mg/dL to about 48 mg/dL, about 1.0 mg/dL to about 45 mg/dL, about 1.0 mg/dL to about 40 mg/dL, about 1.0 mg/dL to about 35 mg/dL, about 1.0 mg/dL to about 30 mg/dL, about 1.0 mg/dL to about 25 mg/dL, about 1.0 mg/dL to about 20 mg/dL, about 1.0 mg/dL to about 18 mg/dL, about 1.0 mg/dL to about 16 mg/dL, about 1.0 mg/dL to about 14 mg/dL, about 1.0 mg/dL to about 12 mg/dL, about 1.0 mg/dL to about 10 mg/dL, about 1.0 mg/dL to about 8 mg/dL, about 1.0 mg/dL to about 6 mg/dL, about 1.0 mg/dL to about 4 mg/dL, about 1.0 mg/dL to about 2 mg/dL, about 2 mg/dL to about 50 mg/dL, about 2 mg/dL to about 48 mg/dL, about 2 mg/dL to about 45 mg/dL, about 2 mg/dL to about 40 mg/dL, about 2 mg/dL to about 35 mg/dL, about 2 mg/dL to about 30 mg/dL, about 2 mg/dL to about 25 mg/dL, about 2 mg/dL to about 20 mg/dL, about 2 mg/dL to about 18 mg/dL, about 2 mg/dL to about 16 mg/dL, about 2 mg/dL to about 14 mg/dL, about 2 mg/dL to about 12 mg/dL, about 2 mg/dL to about 10 mg/dL, about 2 mg/dL to about 8 mg/dL, about 2 mg/dL to about 6 mg/dL, about 2 mg/dL to about 4 mg/dL, about 4 mg/dL to about 50 mg/dL, about 4 mg/dL to about 48 mg/dL, about 4 mg/dL to about 45 mg/dL, about 4 mg/dL to about 40 mg/dL, about 4 mg/dL to about 35 mg/dL, about 4 mg/dL to about 30 mg/dL, about 4 mg/dL to about 25 mg/dL, about 4 mg/dL to about 20 mg/dL, about 4 mg/dL to about 18 mg/dL, about 4 mg/dL to about 16 mg/dL, about 4 mg/dL to about 14 mg/dL, about 4 mg/dL to about 12 mg/dL, about 4 mg/dL to about 10 mg/dL, about 4 mg/dL to about 8 mg/dL, about 4 mg/dL to about 6 mg/dL, about 6 mg/dL to about 50 mg/dL, about 6 mg/dL to about 48 mg/dL, about 6 mg/dL to about 45 mg/dL, about 6 mg/dL to about 40 mg/dL, about 6 mg/dL to about 35 mg/dL, about 6 mg/dL to about 30 mg/dL, about 6 mg/dL to about 25 mg/dL, about 6 mg/dL to about 20 mg/dL, about 6 mg/dL to about 18 mg/dL, about 6 mg/dL to about 16 mg/dL, about 6 mg/dL to about 14 mg/dL, about 6 mg/dL to about 12 mg/dL, about 6 mg/dL to about 10 mg/dL, about 6 mg/dL to about 8 mg/dL, about 8 mg/dL to about 50 mg/dL, about 8 mg/dL to about 48 mg/dL, about 8 mg/dL to about 45 mg/dL, about 8 mg/dL to about 40 mg/dL, about 8 mg/dL to about 35 mg/dL, about 8 mg/dL to about 30 mg/dL, about 8 mg/dL to about 25 mg/dL, about 8 mg/dL to about 20 mg/dL, about 8 mg/dL to about 18 mg/dL, about 8 mg/dL to about 16 mg/dL, about 8 mg/dL to about 14 mg/dL, about 8 mg/dL to about 12 mg/dL, about 8 mg/dL to about 10 mg/dL, about 10 mg/dL to about 50 mg/dL, about 10 mg/dL to about 48 mg/dL, about 10 mg/dL to about 45 mg/dL, about 10 mg/dL to about 40 mg/dL, about 10 mg/dL to about 35 mg/dL, about 10 mg/dL to about 30 mg/dL, about 10 mg/dL to about 25 mg/dL, about 10 mg/dL to about 20 mg/dL, about 10 mg/dL to about 18 mg/dL, about 10 mg/dL to about 16 mg/dL, about 10 mg/dL to about 14 mg/dL, about 10 mg/dL to about 12 mg/dL, about 12 mg/dL to about 50 mg/dL, about 12 mg/dL to about 48 mg/dL, about 12 mg/dL to about 45 mg/dL, about 12 mg/dL to about 40 mg/dL, about 12 mg/dL to about 35 mg/dL, about 12 mg/dL to about 30 mg/dL, about 12 mg/dL to about 25 mg/dL, about 12 mg/dL to about 20 mg/dL, about 12 mg/dL to about 18 mg/dL, about 12 mg/dL to about 16 mg/dL, about 12 mg/dL to about 14 mg/dL, about 14 mg/dL to about 50 mg/dL, about 14 mg/dL to about 48 mg/dL, about 14 mg/dL to about 45 mg/dL, about 14 mg/dL to about 40 mg/dL, about 14 mg/dL to about 35 mg/dL, about 14 mg/dL to about 30 mg/dL, about 14 mg/dL to about 25 mg/dL, about 14 mg/dL to about 20 mg/dL, about 14 mg/dL to about 18 mg/dL, about 14 mg/dL to about 16 mg/dL, about 16 mg/dL to about 50 mg/dL, about 16 mg/dL to about 48 mg/dL, about 16 mg/dL to about 45 mg/dL, about 16 mg/dL to about 40 mg/dL, about 16 mg/dL to about 35 mg/dL, about 16 mg/dL to about 30 mg/dL, about 16 mg/dL to about 25 mg/dL, about 16 mg/dL to about 20 mg/dL, about 16 mg/dL to about 18 mg/dL, about 18 mg/dL to about 50 mg/dL, about 18 mg/dL to about 48 mg/dL, about 18 mg/dL to about 45 mg/dL, about 18 mg/dL to about 40 mg/dL, about 18 mg/dL to about 35 mg/dL, about 18 mg/dL to about 30 mg/dL, about 18 mg/dL to about 25 mg/dL, about 18 mg/dL to about 20 mg/dL, about 20 mg/dL to about 50 mg/dL, about 20 mg/dL to about 48 mg/dL, about 20 mg/dL to about 45 mg/dL, about 20 mg/dL to about 40 mg/dL, about 20 mg/dL to about 35 mg/dL, about 20 mg/dL to about 30 mg/dL, about 20 mg/dL to about 25 mg/dL, about 25 mg/dL to about 50 mg/dL, about 25 mg/dL to about 48 mg/dL, about 25 mg/dL to about 45 mg/dL, about 25 mg/dL to about 40 mg/dL, about 25 mg/dL to about 35 mg/dL, about 25 mg/dL to about 30 mg/dL, about 30 mg/dL to about 50 mg/dL, about 30 mg/dL to about 48 mg/dL, about 30 mg/dL to about 45 mg/dL, about 30 mg/dL to about 40 mg/dL, about 30 mg/dL to about 35 mg/dL, about 35 mg/dL to about 50 mg/dL, about 35 mg/dL to about 48 mg/dL, about 35 mg/dL to about 45 mg/dL, about 35 mg/dL to about 40 mg/dL, about 40 mg/dL to about 50 mg/dL, about 40 mg/dL to about 48 mg/dL, about 40 mg/dL to about 45 mg/dL, about 45 mg/dL to about 50 mg/dL, about 45 mg/dL to about 48 mg/dL, or about 48 mg/dL to about 50 mg/dL) (e.g., for about 6 hours to about one year, or any of the subranges of this range, after the administration of a first dose of the antibody or the antigen-binding fragment to the subject).

F. Combination Therapy

The methods of treatment described herein can be combined with other treatments or the use of other therapeutic agents, such as chemotherapy, radiation, stem cell treatment, surgery other treatments effective against multiple myeloma (MM). Useful classes of other agents that can be administered with any of the pharmaceutical compositions described herein (e.g., including any of the antibodies or antigen-binding fragments described herein) include, for example, antibodies to other receptors expressed on cancerous cells, anti-tubulin agents (e.g., auristatins), DNA minor groove binders (e.g., PBDs), DNA replication inhibitors, alkylating agents (e.g., platinum complexes, such as cis-platin, mono(platinum), bis(platinum), and tri-nuclear platinum complexes, and carboplatin), anthracyclins, antibiotics, antifolates, antimetabolites, chemotherapy sensitizers, duocarmycins, etoposides, fluorinated pyrimidines, ionophores, lexitropsins, nitrosoureas, platinols, pre-forming compounds, purine antimetabolites, puromycins, radiation sensitizers, steroids, taxanes, topoisomerase inhibitors, vinca alkaloids, and the like.

In some embodiments, the anti-BCMA antibody or antigen-binding fragment thereof (e.g., SEA-BCMA) are combined with the use of one or more therapeutic agents selected from dexamethasone, an IMiD agent (e.g., pomalidomide), an anti-CD38 antibody or antigen-binding fragment thereof (e.g., daratumumab), and/or a gamma-secretase inhibitor.

In some embodiments, one or more doses of the anti-BCMA antibody or antigen-binding fragment thereof as described herein (e.g., SEA-BCMA) and one or more doses of dexamethasone are administered to the subject.

In some embodiments, one or more doses of the anti-BCMA antibody or antigen-binding fragment thereof as described herein (e.g., SEA-BCMA), one or more doses of dexamethasone, and one or more doses of an IMiD agent (e.g., pomalidomide) are administered to the subject.

In some embodiments, one or more doses of the anti-BCMA antibody or antigen-binding fragment thereof as described herein (e.g., SEA-BCMA), one or more doses of dexamethasone, one or more doses of an IMiD agent (e.g., pomalidomide), and one or more doses of an anti-CD38 antibody or antigen-binding fragment thereof (e.g., daratumumab) are administered to the subject.

In some embodiments, one or more doses of the anti-BCMA antibody or antigen-binding fragment thereof as described herein (e.g., SEA-BCMA), one or more doses of dexamethasone, one or more doses of an IMiD agent (e.g., pomalidomide), one or more doses of an anti-CD38 antibody or antigen-binding fragment thereof (e.g., daratumumab), and one or more doses of a gamma-secretase inhibitor are administered to the subject.

In some embodiments, one or more doses of the anti-BCMA antibody or antigen-binding fragment thereof as described herein (e.g., SEA-BCMA), one or more doses of dexamethasone, and one or more doses of an anti-CD38 antibody or antigen binding fragment thereof (e.g., daratumumab) are administered to the subject.

In some embodiments, one or more doses of the anti-BCMA antibody or antigen-binding fragment thereof as described herein (e.g., SEA-BCMA), one or more doses of dexamethasone, one or more doses of an anti-CD38 antibody or antigen-binding fragment thereof (e.g., daratumumab), and one or more doses of a gamma-secretase inhibitor are administered to the subject.

In some embodiments, one or more doses of the anti-BCMA antibody or antigen-binding fragment thereof as described herein (e.g., SEA-BCMA), one or more doses of dexamethasone, and one or more doses of a gamma-secretase inhibitor are administered to the subject.

In some embodiments, one or more doses of the anti-BCMA antibody or antigen-binding fragment thereof as described herein (e.g., SEA-BCMA) and one or more doses of an IMiD agent (e.g., pomalidomide) are administered to the subject.

In some embodiments, one or more doses of the anti-BCMA antibody or antigen-binding fragment thereof as described herein (e.g., SEA-BCMA), one or more doses of an IMiD agent (e.g., pomalidomide), and one or more doses of an anti-CD38 antibody or antigen-binding fragment thereof (e.g., daratumumab) are administered to the subject.

In some embodiments, one or more doses of the anti-BCMA antibody or antigen-binding fragment thereof as described herein (e.g., SEA-BCMA), one or more doses of an IMiD agent (e.g., pomalidomide), one or more doses of an anti-CD38 antibody or antigen-binding fragment thereof (e.g., daratumumab), and one or more doses of a gamma-secretase inhibitor are administered to the subject.

In some embodiments, one or more doses of the anti-BCMA antibody or antigen-binding fragment thereof as described herein (e.g., SEA-BCMA), one or more doses of an IMiD agent (e.g., pomalidomide), and one or more doses of a gamma-secretase inhibitor are administered to the subject.

In some embodiments, one or more doses of the anti-BCMA antibody or antigen-binding fragment thereof as described herein (e.g., SEA-BCMA), and one or more doses of an anti-CD38 antibody or antigen-binding fragment thereof (e.g., daratumumab) are administered to the subject.

In some embodiments, one or more doses of the anti-BCMA antibody or antigen-binding fragment thereof as described herein (e.g., SEA-BCMA), one or more doses of an anti-CD38 antibody or antigen-binding fragment thereof (e.g., daratumumab), and one or more doses of a gamma-secretase inhibitor are administered to the subject.

In some embodiments, one or more doses of the anti-BCMA antibody or antigen-binding fragment thereof as described herein (e.g., SEA-BCMA) and one or more doses of a gamma-secretase inhibitor are administered to the subject.

The combination of these therapeutic agents are summarized in the table below.

TABLE 1

| | | | anti-CD38 antibody | |
|---|---|---|---|---|
| SEA- | | IMiD | or antigen binding | |
| BCMA | Dexamethasone | (pomalidomide) | fragment thereof | GSI |
| X | X | | | |
| X | X | X | | |
| X | X | X | X | |
| X | X | X | X | X |
| X | X | | X | |
| X | X | | X | X |
| X | X | | | X |
| X | | X | | |
| X | | X | X | |
| X | | X | X | X |
| X | | X | | X |
| X | | | X | |
| X | | | X | X |
| X | | | | X |

In some embodiments, the anti-BCMA antibody or antigen-binding fragment thereof as described herein (e.g., SEA-BCMA) in these combination therapies can be administered to the subject once every two weeks.

In some embodiments, the anti-BCMA antibody or antigen-binding fragment thereof as described herein (e.g., SEA-BCMA) in these combination therapies can be administered to the subject once every week for the first 8 weeks, and then once every two weeks. For example, the anti-BCMA antibody or antigen-binding fragment thereof as described herein (e.g., SEA-BCMA) can be administered to the subject once every week for two 28-day cycles, and then once every two weeks in the following 28-day cycles.

(i) Combination Therapy with Dexamethasone

In some embodiments, the methods of treatment described herein are combined with the use of dexamethasone. In some embodiments, one or more doses of about 5 mg to about 200 mg (e.g., about 5 mg to about 150 mg, about 5 mg to about 100 mg, about 5 mg to about 90 mg, about 5 mg to about 80 mg, about 5 mg to about 70 mg, about 5 mg to about 60 mg, about 5 mg to about 50 mg, about 5 mg to about 40 mg, about 5 mg to about 30 mg, about 5 mg to about 20 mg; about 10 mg to about 200 mg, about 10 mg to about 150 mg, about 10 mg to about 100 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 30 mg, about 10 mg to about 20 mg, about 20 mg to about 200 mg, about 20 mg to about 150 mg, about 20 mg to about 100 mg, about 20 mg to about 90 mg, about 20 mg to about 80 mg, about 20 mg to about 70 mg, about 20 mg to about 60 mg, about 20 mg to about 50 mg, about 20 mg to about 40 mg) of dexamethasone is independently administered to the subject in combination with the pharmaceutical compositions described herein (e.g., comprising any of the antibodies or antigen-binding fragments described herein). In some embodiments, one or more doses of about 40 mg of dexamethasone is independently administered to the subject in combination with the pharmaceutical compositions described herein (e.g., comprising any of the antibodies or antigen-binding fragments described herein). In some embodiments, one or more doses of about 20 mg of dexamethasone is independently administered to the subject in combination with the pharmaceutical compositions described herein (e.g., comprising any of the antibodies or antigen-binding fragments described herein).

In some embodiments, the dexamethasone is administered to the subject in combination with each and every dose of the pharmaceutical compositions described herein (e.g., comprising any of the antibodies or antigen-binding fragments described herein).

In some embodiments, the dexamethasone is administered to the subject about 10 minutes to about 5 hours (e.g., about 5 minutes to about 4.5 hours, about 5 minutes to about 4 hours, about 5 minutes to about 3.5 hours, about 5 minutes to about 3 hours, about 5 minutes to about 2.5 hours, about 5 minutes to about 2 hours, about 5 minutes to about 1.5 hours, about 5 minutes to about 1 hours, about 5 minutes to about 45 minutes, about 5 minutes to about 40 minutes, about 5 minutes to about 35 minutes, about 5 minutes to about 30 minutes, about 5 minutes to about 25 minutes, about 5 minutes to about 20 minutes, about 5 minutes to about 15 minutes, about 5 minutes to about 10 minutes, about 30 minutes to about 5 hours, about 30 minutes to about 4.5 hours, about 30 minutes to about 4 hours, about 30 minutes to about 3.5 hours, about 30 minutes to about 3 hours, about 30 minutes to about 2.5 hours, about 30 minutes to about 2 hours, about 30 minutes to about 1.5 hours, about 30 minutes to about 1 hours, about 30 minutes to about 45 minutes, about 1 hour to about 5 hours, about 1 hour to about 4.5 hours, about 1 hour to about 4 hours, about 1 hour to about 3.5 hours, about 1 hour to about 3 hours, about 1 hour to about 2.5 hours, about 1 hour to about 2 hours, about 1 hour to about 1.5 hours) prior to the administration of each dose of the pharmaceutical composition described herein (e.g., comprising any of the antibodies or antigen-binding fragments described herein).

In some embodiments, the dexamethasone is administered to the subject about 10 minutes to about 5 hours (e.g., about 5 minutes to about 4.5 hours, about 5 minutes to about 4 hours, about 5 minutes to about 3.5 hours, about 5 minutes to about 3 hours, about 5 minutes to about 2.5 hours, about 5 minutes to about 2 hours, about 5 minutes to about 1.5 hours, about 5 minutes to about 1 hours, about 5 minutes to about 45 minutes, about 5 minutes to about 40 minutes, about 5 minutes to about 35 minutes, about 5 minutes to about 30 minutes, about 5 minutes to about 25 minutes, about 5 minutes to about 20 minutes, about 5 minutes to about 15 minutes, about 5 minutes to about 10 minutes, about 30 minutes to about 5 hours, about 30 minutes to about 4.5 hours, about 30 minutes to about 4 hours, about 30 minutes to about 3.5 hours, about 30 minutes to about 3 hours, about 30 minutes to about 2.5 hours, about 30 minutes to about 2 hours, about 30 minutes to about 1.5 hours, about 30 minutes to about 1 hours, about 30 minutes to about 45 minutes, about 1 hour to about 5 hours, about 1 hour to about 4.5 hours, about 1 hour to about 4 hours, about 1 hour to about 3.5 hours, about 1 hour to about 3 hours, about 1 hour to about 2.5 hours, about 1 hour to about 2 hours, about 1 hour to about 1.5 hours) after the administration of each dose of the pharmaceutical composition described herein (e.g., comprising any of the antibodies or antigen-binding fragments described herein).

In some embodiments, a dose of about 40 mg of dexamethasone is administered to the subject about 1 to about 3 hours prior to each dose of the pharmaceutical composition described herein (e.g., comprising any of the antibodies or antigen-binding fragments described herein).

(ii) Combination Therapy with IMiD agents

Immunomodulatory imide drugs (IMiDs) are immunomodulatory agents containing an “imide” group. Exemplary IMiDs include, but are not limited to, lenalidomide, pomalidomide, thalidomide, and Iberdomide (CC-220, Celgene). The IMiDs can enhance NK cell expansion and activity, and increase antibody-dependent cellular cytotoxicity (ADCC) when combined with monoclonal antibodies.

In some embodiments, the methods of treatment described herein are combined with the use of an IMiD (e.g., pomalidomide). In some embodiments, one or more doses of about 0.5 mg to about 50 mg (e.g., about 1 mg to about 50 mg, about 1 mg to about 40 mg, about 1 mg to about 30 mg, about 1 mg to about 20 mg, about 1 mg to about 10 mg, about 1 mg to about 5 mg, about 2 mg to about 50 mg, about 2 mg to about 40 mg, about 2 mg to about 30 mg, about 2 mg to about 20 mg, about 2 mg to about 10 mg, about 2 mg to about 5 mg, about 3 mg to about 50 mg, about 3 mg to about 40 mg, about 3 mg to about 30 mg, about 3 mg to about 20 mg, about 3 mg to about 10 mg, about 3 mg to about 5 mg, about 4 mg to about 50 mg, about 4 mg to about 40 mg, about 4 mg to about 30 mg, about 4 mg to about 20 mg, about 4 mg to about 10 mg, about 4 mg to about 5 mg, about 1 mg to about 4 mg, about 1 mg to about 6 mg, about 2 mg to about 6 mg, or about 3 mg to about 6 mg) of an IMiD (e.g., pomalidomide) is independently administered to the subject in combination with the pharmaceutical compositions described herein (e.g., comprising any of the antibodies or antigen-binding fragments described herein). In some embodiments, one or more doses of about 4 mg of an IMiD (e.g., pomalidomide) is independently administered (e.g., orally administered) to the subject in combination with the pharmaceutical compositions described herein (e.g., the anti-BCMA antibodies or antigen-binding fragments described herein).

In some embodiments, the IMiD (e.g., pomalidomide) is administered to the subject in combination with each and every dose of the pharmaceutical compositions described herein (e.g., comprising any of the antibodies or antigen-binding fragments described herein). In some embodiments, one or more doses of the antibodies or antigen-binding fragments described herein, one or more doses of dexamethasone, and one or more doses of an IMiD (e.g., pomalidomide) are administered to the subject.

In some embodiments, the IMiD (e.g., pomalidomide) is administered to the subject at a frequency of about once per day to about once every week (e.g., about once per day, about once every two days, about once every three days, about once every four days, about once every five days, about once every six days, or about once every week). In some embodiments, the IMiD (e.g., pomalidomide) is administered to the subject on days 1-21 of repeated 28-day cycles.

In some embodiments, the IMiD (e.g., pomalidomide) is administered to the subject about 10 minutes to about 5 hours (e.g., about 5 minutes to about 4.5 hours, about 5 minutes to about 4 hours, about 5 minutes to about 3.5 hours, about 5 minutes to about 3 hours, about 5 minutes to about 2.5 hours, about 5 minutes to about 2 hours, about 5 minutes to about 1.5 hours, about 5 minutes to about 1 hours, about 5 minutes to about 45 minutes, about 5 minutes to about 40 minutes, about 5 minutes to about 35 minutes, about 5 minutes to about 30 minutes, about 5 minutes to about 25 minutes, about 5 minutes to about 20 minutes, about 5 minutes to about 15 minutes, about 5 minutes to about 10 minutes, about 30 minutes to about 5 hours, about 30 minutes to about 4.5 hours, about 30 minutes to about 4 hours, about 30 minutes to about 3.5 hours, about 30 minutes to about 3 hours, about 30 minutes to about 2.5 hours, about 30 minutes to about 2 hours, about 30 minutes to about 1.5 hours, about 30 minutes to about 1 hours, about 30 minutes to about 45 minutes, about 1 hour to about 5 hours, about 1 hour to about 4.5 hours, about 1 hour to about 4 hours, about 1 hour to about 3.5 hours, about 1 hour to about 3 hours, about 1 hour to about 2.5 hours, about 1 hour to about 2 hours, about 1 hour to about 1.5 hours) prior to the administration of each dose of the pharmaceutical composition described herein (e.g., comprising any of the antibodies or antigen-binding fragments described herein). In some embodiments, the IMiD (e.g., pomalidomide) is administered to the subject about 1 hour to about 3 hours prior to the administration of the anti-BCMA antibody or antigen-binding fragment thereof as described herein.

(iii) Combination Therapy with an Anti-CD38 Antibody

CD38, also known as cyclic ADP ribose hydrolase is a glycoprotein that are expressed on the surface of many immune cells, including CD4+, CD8+, B lymphocytes and natural killer cells. CD38 is overexpressed in multiple myeloma cells. An anti-CD38 antibody or antigen-binding fragments thereof (e.g., daratumumab) can bind to CD38, causing cells to induce apoptosis via antibody-dependent cellular cytotoxicity, complement-dependent cytotoxicity, inhibition of mitochondrial transfer or antibody-dependent cellular phagocytosis. As shown in the examples below, a BCMA therapeutic agent can be effectively combined with an anti-CD38 antibody or antigen-binding fragment thereof. Thus, in some embodiments, a BCMA therapeutic agent (e.g., an anti-BCMA antagonist antibody, an anti-BCMA ADC (e.g., belantamab mafodotin), BCMA-targeted chimeric antigen receptor T-cell (CAR-T therapy), a BCMA bispecific antibody, or a BCMA bispecific T cell engager (BiTE)) is combined with an anti-CD38 antibody or antigen-binding fragment thereof. In some embodiments, the BCMA therapeutic agent is a non-fucosylated BCMA antibody, such as the SEA-BCMA antibody described herein.

In some embodiments, the BCMA therapeutic agent is an antibody-drug conjugate, e.g., Belantamab mafodotin (GSK2857916), MEDI2228, or HDP-101. In some embodiments, the BCM therapeutic agent is CAR-T, e.g., CAR-BCMA (NCI), Idecabtagene Vicleucel (Bb2121), Bb21217, LCAR-B38M, JNJ-4528, CT053, P-BCMA-101, CART-BCMA (UPenn/Novartis), CT103A, JCARH125, MCARH171, BCMA CAR-T (HRAIN Biotech), or KITE-585. In some embodiments, the BCMA therapeutic agent is a bispecific T cell engager (BiTE) or trispecific T cell engager (TiTE), e.g., AMG 420 (Amgen), CC-93269 (Celgene), PF-06863135 (Pfizer), REGN5458 (Regeneron), AMG 701 (Amgen), or TNB383B (TeneoBio). Specific examples of such agents are described, for example, in Yu et al. (2020) J. Hematology & Oncology 13:125, which is incorporated by reference in its entirety.

In some embodiments, the anti-CD38 antibody or antigen-binding fragment thereof is daratumumab. Daratumumab is described e.g., in U.S. Pat. No. 7,829,693, and US20170121414A1, both of which are incorporated herein by the reference in the entirety. In some embodiments, the anti-CD38 antibody or antigen-binding fragment thereof is MOR03087 (MOR202), JNJ-54767414 (HuMax CD38), lsatuximab SAR650984, Ab79, etc. Many of these anti-CD38 antibodies or antigen-binding fragments are described e.g., in U.S. Ser. No. 10/494,444B2, WO2020212914A1, Morandi, Fabio, et al. Frontiers in immunology 9 (2018): 2722; Lammerts van Bueren, Jeroen, et al. (2014): 3474-3474; Pillai, Rathi N., et al. JTO Clinical and Research Reports 2.2 (2021): 100104; and Martin, Thomas, et al. (2015): 509-509; each of which is incorporated herein by reference in its entirety.

In some embodiments, the methods of treatment described herein are combined with the use of an anti-CD38 antibody or antigen-binding fragments thereof (e.g., daratumumab). In some embodiments, one or more doses of about 1 mg/kg to about 100 mg/kg (e.g., about 1 mg/kg to about 50 mg/kg; about 4 mg/kg to about 30 mg/kg; about 10 mg/kg to about 50 mg/kg; about 10 mg/kg to about 30 mg/kg; about 10 mg/kg to about 20 mg/kg; or about 15 mg/kg to about 20 mg/kg) of an anti-CD38 antibody or antigen binding fragments thereof (e.g., daratumumab) is independently administered to the subject in combination with the pharmaceutical compositions described herein (e.g., comprising the anti-BCMA antibodies or antigen-binding fragments described herein). In some embodiments, the dosage for the anti-CD38 antibody or antigen-binding fragments thereof (e.g., daratumumab) can be less than 100 mg/kg, less than 20 mg/kg, less than 19 mg/kg, less than 18 mg/kg, less than 17 mg/kg, less than 16 mg/kg, less than 15 mg/kg, less than 14 mg/kg, less than 13 mg/kg, less than 12 mg/kg, less than 11 mg/kg, less than 10 mg/kg, less than 5 mg/kg, or less than 1 mg/kg. In some embodiments, the dosage for the anti-CD38 antibody or antigen-binding fragments thereof (e.g., daratumumab) can be greater than 10 mg/kg, greater than 9 mg/kg, greater than 8 mg/kg, greater than 7 mg/kg, greater than 6 mg/kg, greater than 5 mg/kg, greater than 4 mg/kg, greater than 3 mg/kg, greater than 2 mg/kg, greater than 1 mg/kg, greater than 0.5 mg/kg, or greater than 0.1 mg/kg. In some embodiments, the dosage for the anti-CD38 antibody or antigen-binding fragments thereof (e.g., daratumumab) can be about 1 mg/kg. It can be dosed weekly, e.g., for about 5 weeks.

In some embodiments, one or more doses of about 16 mg/kg of daratumumab is independently administered (e.g., orally administered) to the subject in combination with the pharmaceutical compositions described herein (e.g., comprising the anti-BCMA antibodies or antigen-binding fragments described herein).

In some embodiments, a combination of the anti-BCMA antibody or antigen-binding fragment thereof, the anti-CD38 antibody or antigen-binding fragment thereof, and dexamethasone are administered to the subject. In some embodiments, the combination of the anti-BCMA antibody or antigen-binding fragment thereof, the anti-CD38 antibody or antigen-binding fragment thereof, and an IMiD are administered to the subject. In some embodiments, the combination of the anti-BCMA antibody or antigen-binding fragment thereof, the anti-CD38 antibody or antigen-binding fragment thereof, dexamethasone, and an IMiD are administered to the subject.

In some embodiments, the anti-CD38 antibody or antigen-binding fragment thereof (e.g., daratumumab) are administered to the subject at a frequency of about once a week to about once every four weeks (e.g., about once a week, about once two weeks, about once three weeks, or about once four weeks). In some embodiments, the anti-CD38 antibody or antigen-binding fragment thereof are administered to the subject on day 1, day 8, day 15 and day 22 for 28-day cycles.

In some embodiments, the anti-CD38 antibody or antigen-binding fragment thereof are administered to the subject at a frequency of about once every week during a first phase. In some embodiments, the first phase is about 2 weeks to about 20 weeks (e.g., about 2 weeks to about 10 weeks, about 4 weeks to about 20 weeks, about 4 weeks to about 10 weeks, about 6 weeks to about 20 weeks, about 6 weeks to about 10 weeks, or about 7 weeks to about 9 weeks). In some embodiments, the first phase is about or at least 2, 3, 4, 5, 6, 7, 8, 9, 10, 11, 12, 13, 14, 15, 16, 17, 18, 19, or 20 weeks.

In some embodiments, the anti-CD38 antibody or antigen-binding fragment thereof are optionally administered to the subject at a frequency of about once every two weeks to about once every three weeks during a second phase. In some embodiments, the second phase is about 2 weeks to about 24 weeks (e.g., about 5 weeks to about 20 weeks, about 10 weeks to about 20 weeks, about 15 weeks to about 20 weeks, or about 15 weeks to about 16 weeks). In some embodiments, the second phase is about or at least 2, 3, 4, 5, 6, 7, 8, 9, 10, 11, 12, 13, 14, 15, 16, 17, 18, 19, or 20 weeks.

In some embodiments, the anti-CD38 antibody or antigen-binding fragment thereof are optionally administered to the subject at a frequency of about once every four weeks during a third phase. In some embodiments, the third phase is about 2 weeks to about 30 weeks (e.g., about 2 weeks to about 10 weeks, about 4 weeks to about 20 weeks, about 4 weeks to about 10 weeks, about 6 weeks to about 20 weeks, about 6 weeks to about 10 weeks, or about 7 weeks to about 9 weeks). In some embodiments, the third phase is about or at least 2, 3, 4, 5, 6, 7, 8, 9, 10, 11, 12, 13, 14, 15, 16, 17, 18, 19, 20, 25, or 30 weeks. In some embodiments, the anti-CD38 antibody or antigen-binding fragment are continuously administered to the subject until the disease is cured or the subject no longer responds to the treatment.

In some embodiments, the anti-CD38 antibody or antigen-binding fragment is administered to the subject at a frequency of once a week from weeks 1 to 8, at a frequency of once every two weeks from weeks 9 to 24, and then at a frequency of once every four weeks from week 25 onwards until disease progression.

In some embodiments, the anti-CD38 antibody or antigen-binding fragment is administered to the subject at a frequency of once a week from weeks 1 to 9, at a frequency of once every three weeks from weeks 10 to 24, and then at a frequency of once every four weeks from week 25 onwards until disease progression.

(iv) Combination Therapy with a Gamma-Secretase Inhibitor

BCMA can be cleaved from the tumor cell surface by the ubiquitous γ-secretase (GS) complex. The cleavage reduces BCMA density on tumor cells for anti-BCMA antibody recognition and releases a soluble BCMA (sBCMA) fragment capable of interfering with the binding of the anti-BCMA antibody to BCMA on the tumor cell surface. Gamma secretase inhibitors (GSIs) can block BCMA cleavage and thus increase BCMA target density on the surface of cells and decrease the amount of sBCMA in the plasma. Furthermore, GSIs can increase BCMA NF-kB signaling.

The combination of the anti-BCMA antibody or antigen binding fragment thereof with a GSI can increase the efficacy of the anti-BCMA antibody or antigen binding fragment thereof. In some embodiments, the GSI is Semagacestat (LY450139), R04929097, MK-0752, Avagacestat (BMS-708163), Nirogacestat (PF-03084014), Crenigacestat (LY3039478), BMS-906024, DAPT (GSI-IX), Dibenzazepine (YO-01027), LY411575, L-685,458, NGP 555, MDL-28170, or Itanapraced (CHF 5074).

In some embodiments, the GSI is administered to the subject in combination with each and every dose of the pharmaceutical compositions described herein (e.g., comprising any of the antibodies or antigen-binding fragments described herein). In some embodiments, one or more doses of GSI are administered to the subject.

In some embodiments, a combination of the anti-BCMA antibody or antigen-binding fragment thereof and a GSI are administered to the subject. In some embodiments, dexamethasone, an anti-CD38 antibody or antigen-binding fragment thereof ad described herein, and/or an IMiD further are further administered to the subject.

In addition, gamma secretase inhibition can induce increased NF-κB signaling, which may have undesirable effects. The anti-BCMA antibody or antigen-binding fragment thereof as described herein (e.g., SEA-BCMA) can partially inhibit the increased NF-κB signaling, thereby further inhibiting tumor growth and/or controlling myeloma progression.

In some embodiments, the GSI is administered to the subject about 10 minutes to about 5 hours (e.g., about 5 minutes to about 4.5 hours, about 5 minutes to about 4 hours, about 5 minutes to about 3.5 hours, about 5 minutes to about 3 hours, about 5 minutes to about 2.5 hours, about 5 minutes to about 2 hours, about 5 minutes to about 1.5 hours, about 5 minutes to about 1 hours, about 5 minutes to about 45 minutes, about 5 minutes to about 40 minutes, about 5 minutes to about 35 minutes, about 5 minutes to about 30 minutes, about 5 minutes to about 25 minutes, about 5 minutes to about 20 minutes, about 5 minutes to about 15 minutes, about 5 minutes to about 10 minutes, about 30 minutes to about 5 hours, about 30 minutes to about 4.5 hours, about 30 minutes to about 4 hours, about 30 minutes to about 3.5 hours, about 30 minutes to about 3 hours, about 30 minutes to about 2.5 hours, about 30 minutes to about 2 hours, about 30 minutes to about 1.5 hours, about 30 minutes to about 1 hours, about 30 minutes to about 45 minutes, about 1 hour to about 5 hours, about 1 hour to about 4.5 hours, about 1 hour to about 4 hours, about 1 hour to about 3.5 hours, about 1 hour to about 3 hours, about 1 hour to about 2.5 hours, about 1 hour to about 2 hours, about 1 hour to about 1.5 hours) prior to or after the administration of each dose of the pharmaceutical composition described herein (e.g., comprising any of the anti-BCMA antibodies or antigen-binding fragments described herein).

G. Therapeutic Effects

The therapeutic effects of the methods described herein can be assessed by the expression levels of one or more biomarkers in a patient sample. Exemplary biomarker assessments include testing the levels of serum free light chain and modified serum protein electrophoresis tests (SPEP), peripheral blood immunophenotyping, such as flow cytometry measurements included, but not be limited to, characterizing NK cells, monocytes, T cells, and B cells, assessment of levels of circulating soluble BCMA (sBCMA), a proliferation-inducing ligand (APRIL) and B-cell activation factor (BAFF), retrospective analyses of cellular and circulating biomarkers, characterization of tumor tissue, bone marrow immunotyping, baseline and treatment-related changes in gene expression profiles in tumor and tumor microenvironment assessed by RNA sequencing in tumor and non-tumor cells, and assessment of levels of soluble target, ligands, and/or cytokines/chemokines in bone marrow plasma.

The therapeutic effects achieved by the methods described herein can also include, for example, a decrease in severity of disease symptoms, an increase in frequency and duration of disease symptom-free periods, an increase in lifespan, disease remission, or a prevention of impairment or disability due to the disease affliction. For example, for the treatment of multiple myeloma, aggressive and/or drug resistant and/or refractory multiple myeloma, the methods described herein inhibits cell growth or tumor growth by at least about 20%, at least about 30%, at least about 40%, at least about 50%, at least about 60%, at least about 70%, at least about 80%, at least about 90%, or at least about 95%, relative to untreated subjects or subjects receiving a different treatment. In addition, the methods described herein can result in at least stable disease, partial response, or complete response, as assessed by the WHO or RECIST criteria for tumor response (Natl. Cancer. Inst. 91:523-8, 1999; and Cancer 47:207-14, 1981). In some embodiments, a treatment effect is determined on the basis of an objective response, objective response rate, complete response, complete response rate, duration of response, duration of complete response, progression free survival, and overall survival.

The methods described herein can decrease tumor size or cancer burden, or otherwise ameliorate symptoms in a subject, or otherwise support partial or complete stable disease and/or partial or complete response as determined above.

Treatment with any of the pharmaceutical compositions described herein (e.g., comprising any of the antibodies or antigen-binding fragments described herein), optionally in combination with any of the other therapeutic agents or treatments described herein, can increase the median progression-free survival or overall survival time of patients with cancer, especially when relapsed or refractory, by at least 30%, at least 40%, at least 50%, at least 60%, at least 70%, at least 80%, at least 90%, or at least 95%, compared to the same treatment (e.g., chemotherapy) but without administration of any of the pharmaceutical compositions comprising any of the anti-BCMA antibodies or antigen-binding fragments described herein. In addition or alternatively, treatment (e.g., standard chemotherapy) including administration of any of the pharmaceutical compositions comprising any of the anti-BCMA antibodies or antigen-binding fragments described herein, can increase the complete response rate, partial response rate, or objective response rate (complete+partial) of patients with tumors by at least 30%, at least 40%, at least 50%, at least 60%, at least 70%, at least 80%, at least 90%, or at least 95%, compared to the same treatment (e.g., chemotherapy) but without administration of any of the pharmaceutical compositions comprising any of the anti-BCMA antibodies or antigen-binding fragments described herein.

Typically, in a clinical trial (e.g., a phase II, phase II/III, or phase III trial), the aforementioned increases in median progression-free survival and/or response rate of the patients treated with standard therapy plus any of the pharmaceutical compositions comprising any of the anti-BCMA antibodies or antigen-binding fragments described herein, relative to the control group of patients receiving standard therapy alone (or plus placebo), are statistically significant, for example at the p=0.05, 0.01, or 0.001 level. The complete and partial response rates are determined by objective criteria commonly used in clinical trials for cancer, e.g., as listed or accepted by the National Cancer Institute and/or Food and Drug Administration.

A patient is determined to have an objective response (OR) if, based on the 2016 IMWG uniform response criteria, they achieve a stringent complete response (sCR), complete response (CR), very good partial response (VGPR), or a partial response (PR). The objective response rate (ORR) is defined as the proportion of patients with an OR per investigator. Patients whose disease response cannot be evaluated per the 2016 IMWG uniform response criteria are scored as Not Evaluable for calculating the ORR. Patients who do not have post baseline response assessment, or the response is Not Evaluable per IMWG criteria are counted as non-responders in calculation of ORR. Objective response (OR) can be assessed by imaging, laboratory assessment, or physical examination; or SD and clinical improvement in disease-related symptoms per investigator.

In one embodiment of any of the methods described herein, the objective response rate (ORR) is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% after the administration of the antibodies or antigen-binding fragments described herein.

A patient is determined to have a complete response (CR) if, based on the 2016 IMWG uniform response criteria they achieve a sCR or CR. The CR rate is defined as the proportion of patients with a CR per investigator. Patients whose disease response cannot be evaluated per the IMWG uniform response criteria are scored as Not Evaluable for calculating the CR rate.

In one embodiment of any of the methods described herein, the complete response rate (CRR) is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% after the administration of the antibodies or antigen-binding fragments described herein.

Duration of OR is defined as the time from first documentation of OR (sCR, CR, VGPR, or PR) to the first documentation of disease progression or to death due to any cause, whichever comes first. Disease progression includes objective evidence of tumor progression (based on serum, urine, or bone marrow assessments) and/or clinical progression per investigator. Duration of response is only calculated for the subgroup of patients achieving a sCR, CR, VGPR, or PR.

In one embodiment of any of the methods described herein, the duration of objective response or the duration of complete response to the treatment is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years.

Progression-free survival (PFS) is defined as the time from the start of treatment to first documentation of disease progression or to death due to any cause, whichever comes first. Disease progression includes objective evidence of tumor progression (based on serum, urine or bone marrow assessments) and/or clinical progression per investigator. PFS is censored on the date of the last disease assessment documenting absence of progressive disease (PD) for patients who do not have disease progression and are still on study at the time of an analysis, or are removed from study prior to documentation of tumor progression. Patients who have started a new antitumor treatment prior to documentation of PD will be censored at the last disease assessment prior to start of new treatment. Patients lacking an evaluation of tumor response after their first dose have their event time censored at 1 day.

In one embodiment of any of the methods described herein, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years.

Overall survival (OS) is defined as the time from the start of any study treatment to the date of death due to any cause. Specifically,

OS=date of death−date of first dose of any study treatment+1.

In one embodiment of any of the methods described herein, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years.

H. Exemplary Monotherapies and Combination Therapies

In certain embodiments, the subject receives monotherapy once every two weeks (q2wk) according to a standard dosing regimen. In some of the standard dosing monotherapy treatments, each dose contains 800 mg of an anti-BCMA antibody or an antigen-binding fragment described herein. In other standard dosing monotherapy treatments, each dose administered to the subject contains 1600 mg of an anti-BCMA antibody or an antigen-binding fragment described herein.

In some embodiments of any of the methods described herein, a monotherapy intensive dosing is performed. In certain embodiments, the monotherapy intensive dosing comprises weekly induction dosing (q1wk) of any of the anti-BCMA antibodies or antigen-binding fragments described herein for 8 doses during the first 2 cycles of therapy (i.e. Cycle 1 and Cycle 2). Assuming the patient does not experience confirmed disease progression, the subject is administered any of the anti-BCMA antibodies or antigen-binding fragments described herein dosed q2wk during a maintenance phase during Cycle 3 and beyond. Dosing during the maintenance phase is typically at the standard monotherapy dosing level, i.e., either 800 mg or 1600 mg of the antibody or antigen-binding fragment.

Thus, in some embodiments, intensive dosing monotherapy includes administering 800 or 1600 mg of an anti-BCMA antibody or an antigen-binding fragment described herein, on Day 1, Day 8, Day 15, and Day 22 of Cycle 1 and Cycle 2, and Day 1 and Day 15 of subsequent cycles.

In some embodiments, dexamethasone is combined with the standard or intensive monotherapy regimens as part of a combination therapy. In some embodiments of such combination treatments, dexamethasone is administered as a 40 mg dose and is administered once a week (i.e., q1wk). Thus, for example, some combination therapy embodiments involve a standard dosing combination therapy in which dexamethasone is administered in combination with a standard dosing regimen of the anti-BCMA antibodies or antigen-binding fragments described herein in which the antibody or antigen-binding fragment is administered q2wk. For example, in some standard dosing combination treatments, an anti-BCMA antibody or antigen-binding fragment as described herein is administered on Day 1 and Day 15 of each 28-day cycle (i.e., according to a standard dosing regimen) and dexamethasone is administered on Day 1, Day 8, Day 15, and Day 22 of each 28-day cycle. In some of these standard dosing combination embodiments, each dose of the antibody or antigen binding fragment is administered as an 800 mg dose and each dose of dexamethasone is administered as a 40 mg dose. In other of these embodiments, each dose of the antibody or antigen binding fragment is administered as an 1600 mg dose and each dose of dexamethasone is administered as a 40 mg dose.

Other examples of combination therapy embodiments involve an intensive dosing combination therapy in which dexamethasone is administered in combination with an intensive dose regimen of any of the anti-BCMA antibodies or antigen-binding fragments described herein in which the antibody or antigen-binding fragment is administered q1wk for 8 weeks, followed by q2wk dosing. For example, in some intensive dosing combinations, the anti-BCMA antibody or antigen-binding fragment as described herein is administered on Day 1, Day 8, Day 15, and Day 22 of Cycles 1 and 2, and Day 1 and Day 15 of subsequent cycles (i.e., according to an intensive dosing regimen) and dexamethasone is administered on Day 1, Day 8, Day 15, and Day 22 of each 28-day cycle. In some of these intensive dosing combination embodiments, each dose of the antibody or antigen binding fragment is administered as an 800 mg dose and each dose of dexamethasone is administered as a 40 mg dose. In other of these embodiments, each dose of the antibody or antigen binding fragment is administered as an 1600 mg dose and each dose of dexamethasone is administered as a 40 mg dose.

In any one of the exemplary combination therapies, when the anti-BCMA antibody or antigen-binding fragment and dexamethasone are both administered on the same day, dexamethasone is administered 1 to 3 hours prior to SEA BCMA infusion.

In some embodiments, pomalidomide is further combined with the standard or intensive monotherapy regimens or the combination therapy as described herein. In some embodiments, pomalidomide is administered as a 4 mg dose and is administered daily on days 1-21 of the repeated 28-day cycles.

In some embodiments, the anti-BCMA antibody or an antigen-binding fragment described herein is administered to the subject once every two weeks (e.g., Day 1 and Day 15 of each 28-day cycle), dexamethasone is administered once every week (e.g., on Day 1, Day 8, Day 15, and Day 22 of each 28-day cycle) and pomalidomide is administered daily on days 1-21 of the repeated 28-day cycles. In some embodiments, 1600 mg of an anti-BCMA antibody (e.g., SEA-BCMA) is administered to the subject once every two weeks (e.g., Day 1 and Day 15 of each 28-day cycle), 40 mg of dexamethasone is administered once every week (e.g., on Day 1, Day 8, Day 15, and Day 22 of each 28-day cycle) and 4 mg of pomalidomide is administered daily on days 1-21 of the repeated 28-day cycles.

In some embodiments, the anti-BCMA antibody or an antigen-binding fragment described herein is administered to the subject once every week for about 8 weeks and then once every two weeks (e.g., on Day 1, Day 8, Day 15, and Day 22 of two 28-day cycles, and Day 1 and Day 15 of subsequent 28-day cycles), dexamethasone is administered once every week (e.g., on Day 1, Day 8, Day 15, and Day 22 of each 28-day cycle) and pomalidomide is administered daily on days 1-21 of the repeated 28-day cycles. In some embodiments, 1600 mg of an anti-BCMA antibody (e.g., SEA-BCMA) is administered to the subject once every week for about 8 weeks and then once every two weeks (e.g., on Day 1, Day 8, Day 15, and Day 22 of two 28-day cycles, and Day 1 and Day 15 of subsequent 28-day cycles), 40 mg of dexamethasone is administered once every week (e.g., on Day 1, Day 8, Day 15, and Day 22 of each 28-day cycle) and 4 mg of pomalidomide is administered daily on days 1-21 of the repeated 28-day cycles.

In some embodiments, the anti-BCMA antibody or an antigen-binding fragment described herein is administered to the subject once every two weeks (e.g., Day 1 and Day 15 of each 28-day cycle), and an anti-CD38 antibody or antigen-binding fragment thereof is administered about once every week to about once every four weeks (e.g., about once every week, about once every two weeks, or about once every four weeks). In some embodiments, 1600 mg of an anti-BCMA antibody (e.g., SEA-BCMA) is administered to the subject once every two weeks (e.g., Day 1 and Day 15 of each 28-day cycle), and 16 mg/kg of an anti-CD38 antibody (e.g., daratumumab) is administered about once every week to about once every four weeks (e.g., about once every week, about once every two weeks, or about once every four weeks).

In some embodiments, the anti-BCMA antibody or an antigen-binding fragment described herein is administered to the subject once every week for about 8 weeks and then once every two weeks (e.g., on Day 1, Day 8, Day 15, and Day 22 of two 28-day cycles, and Day 1 and Day 15 of subsequent 28-day cycles), and an anti-CD38 antibody or antigen-binding fragment thereof is administered about once every week to about once every four weeks (e.g., about once every week, about once every two weeks, or about once every four weeks). In some
embodiments, 1600 mg of an anti-BCMA antibody (e.g., SEA-BCMA) is administered to the subject once every week for about 8 weeks and then once every two weeks (e.g., on Day 1, Day 8, Day 15, and Day 22 of two 28-day cycles, and Day 1 and Day 15 of subsequent 28-day cycles), and 16 mg/kg of an anti-CD38 antibody (e.g., daratumumab) is administered about once every week to about once every four weeks (e.g., about once every week, about once every two weeks, or about once every four weeks).

In some embodiments, the anti-BCMA antibody or an antigen-binding fragment described herein is administered to the subject once every two weeks (e.g., Day 1 and Day 15 of each 28-day cycle), and a GSI is administered to the subject with a dosing schedule as appropriately determined for the subject. In some embodiments, 1600 mg of an anti-BCMA antibody (e.g., SEA-BCMA) is administered to the subject once every two weeks (e.g., Day 1 and Day 15 of each 28-day cycle), and a GSI is administered to the subject with a dosing schedule as appropriately determined for the subject.

In some embodiments, the anti-BCMA antibody or an antigen-binding fragment described herein is administered to the subject once every week for about 8 weeks and then once every two weeks (e.g., on Day 1, Day 8, Day 15, and Day 22 of two 28-day cycles, and Day 1 and Day 15 of subsequent 28-day cycles), and a GSI is administered to the subject with a dosing schedule as appropriately determined for the subject. In some embodiments, 1600 mg of an anti-BCMA antibody (e.g., SEA-BCMA) is administered to the subject once every week for about 8 weeks and then once every two weeks (e.g., on Day 1, Day 8, Day 15, and Day 22 of two 28-day cycles, and Day 1 and Day 15 of subsequent 28-day cycles), and a GSI is administered to the subject with a dosing schedule as appropriately determined for the subject.

In some embodiments, the anti-BCMA antibody or an antigen-binding fragment described herein is administered to the subject once every two weeks (e.g., Day 1 and Day 15 of each 28-day cycle), dexamethasone is administered once every week (e.g., on Day 1, Day 8, Day 15, and Day 22 of each 28-day cycle) and an IMiD is administered to the subject with a dosing schedule as appropriately determined for the subject. In some embodiments, 1600 mg of an anti-BCMA antibody (e.g., SEA-BCMA) is administered to the subject once every two weeks (e.g., Day 1 and Day 15 of each 28-day cycle), 40 mg of dexamethasone is administered once every week (e.g., on Day 1, Day 8, Day 15, and Day 22 of each 28-day cycle) and an IMiD is administered to the subject with a dosing schedule as appropriately determined for the subject.

In some embodiments, the anti-BCMA antibody or an antigen-binding fragment described herein is administered to the subject once every week for about 8 weeks and then once every two weeks (e.g., on Day 1, Day 8, Day 15, and Day 22 of two 28-day cycles, and Day 1 and Day 15 of subsequent 28-day cycles), dexamethasone is administered once every week (e.g., on Day 1, Day 8, Day 15, and Day 22 of each 28-day cycle) and an IMiD is administered to the subject with a dosing schedule as appropriately determined for the subject. In some embodiments, 1600 mg of an anti-BCMA antibody (e.g., SEA-BCMA) is administered to the subject once every week for about 8 weeks and then once every two weeks (e.g., on Day 1, Day 8, Day 15, and Day 22 of two 28-day cycles, and Day 1 and Day 15 of subsequent 28-day cycles), 40 mg of dexamethasone is administered once every week (e.g., on Day 1, Day 8, Day 15, and Day 22 of each 28-day cycle) and an IMiD is administered to the subject with a dosing schedule as appropriately determined for the subject.

In some embodiments, the anti-BCMA antibody or an antigen-binding fragment described herein is administered to the subject once every two weeks (e.g., Day 1 and Day 15 of each 28-day cycle), dexamethasone is administered once every week (e.g., on Day 1, Day 8, Day 15, and Day 22 of each 28-day cycle) and an anti-CD38 antibody or antigen-binding fragment thereof is administered about once every week to about once every four weeks (e.g., about once every week, about once every two weeks, or about once every four weeks). In some embodiments, 1600 mg of an anti-BCMA antibody (e.g., SEA-BCMA) is administered to the subject once every two weeks (e.g., Day 1 and Day 15 of each 28-day cycle), 40 mg of dexamethasone is administered once every week (e.g., on Day 1, Day 8, Day 15, and Day 22 of each 28-day cycle) and 16 mg/kg of an anti-CD38 antibody (e.g., daratumumab) is administered about once every week to about once every four weeks (e.g., about once every week, about once every two weeks, or about once every four weeks).

In some embodiments, the anti-BCMA antibody or an antigen-binding fragment described herein is administered to the subject once every week for about 8 weeks and then once every two weeks (e.g., on Day 1, Day 8, Day 15, and Day 22 of two 28-day cycles, and Day 1 and Day 15 of subsequent 28-day cycles), dexamethasone is administered once every week (e.g., on Day 1, Day 8, Day 15, and Day 22 of each 28-day cycle) and an anti-CD38 antibody or antigen-binding fragment thereof is administered about once every week to about once every four weeks (e.g., about once every week, about once every two weeks, or about once every four weeks). In some embodiments, 1600 mg of an anti-BCMA antibody (e.g., SEA-BCMA) is administered to the subject once every week for about 8 weeks and then once every two weeks (e.g., on Day 1, Day 8, Day 15, and Day 22 of two 28-day cycles, and Day 1 and Day 15 of subsequent 28-day cycles), 40 mg of dexamethasone is administered once every week (e.g., on Day 1, Day 8, Day 15, and Day 22 of each 28-day cycle) and 16 mg/kg of an anti-CD38 antibody (e.g., daratumumab) is administered about once every week to about once every four weeks (e.g., about once every week, about once every two weeks, or about once every four weeks).

In some embodiments, the anti-BCMA antibody or an antigen-binding fragment described herein is administered to the subject once every two weeks (e.g., Day 1 and Day 15 of each 28-day cycle), dexamethasone is administered once every week (e.g., on Day 1, Day 8, Day 15, and Day 22 of each 28-day cycle) and a GSI is administered to the subject with a dosing schedule as appropriately determined for the subject. In some embodiments, 1600 mg of an anti-BCMA antibody (e.g., SEA-BCMA) is administered to the subject once every two weeks (e.g., Day 1 and Day 15 of each 28-day cycle), 40 mg of dexamethasone is administered once every week (e.g., on Day 1, Day 8, Day 15, and Day 22 of each 28-day cycle) and a GSI is administered to the subject with a dosing schedule as appropriately determined for the subject.

In some embodiments, the anti-BCMA antibody or an antigen-binding fragment described herein is administered to the subject once every week for about 8 weeks and then once every two weeks (e.g., on Day 1, Day 8, Day 15, and Day 22 of two 28-day cycles, and Day 1 and Day 15 of subsequent 28-day cycles), dexamethasone is administered once every week (e.g., on Day 1, Day 8, Day 15, and Day 22 of each 28-day cycle) and a GSI is administered to the subject with a dosing schedule as appropriately determined for the subject. In some embodiments, 1600 mg of an anti-BCMA antibody (e.g., SEA-BCMA) is administered to the subject once every week for about 8 weeks and then once every two weeks (e.g., on Day 1, Day 8, Day 15, and Day 22 of two 28-day cycles, and Day 1 and Day 15 of subsequent 28-day cycles), 40 mg of dexamethasone is administered once every week (e.g., on Day 1, Day 8, Day 15, and Day 22 of each 28-day cycle) and a GSI is administered to the subject with a dosing schedule as appropriately determined for the subject.

I. Patient selection for different dosing regimens and combination therapies.

The diagnosis of multiple myeloma (MM) requiring systemic therapy can be based on International Myeloma Working Group (IMWG) 2014 criteria. The measurable disease can be defined by one or more of the following:
a) Serum monoclonal paraprotein (M-protein) level ≥0.5 g/dL; for IgA or IgD myeloma patients, serum IgA or serum IgD ≥0.5 g/dL is acceptable
b) Urine M-protein level ≥200 mg/24 hr
c) Serum immunoglobulin FLC ≥10 mg/dL and abnormal serum immunoglobulin kappa lambda FLC ratio

In some embodiments, an ECOG Performance Status score of 0 or 1 is needed before receiving the treatment as described herein.

In some embodiments, hematologic criteria must be met in the absence of growth factor or platelet transfusion support:
a) Estimated glomerular filtration rate (eGFR) ≥30 mL/min/1.73 m² per the Modification of Diet in Renal Disease (MDRD) equation.
b) Absolute neutrophil count ≥1000/μL
c) Platelet count ≥75,000/μL.

Patients can be selected for different dosing regimens or combination therapies. For example, standard dosing (e.g., q2wk, day 1 and day 15 of each 28-day cycle) can be administered to certain patients. In some embodiments, these patients must not have other therapeutic options known to provide clinical benefit in MM available. In some embodiments, patients' prior lines of therapy for patients must include at least a proteasome inhibitor (PI), an immunomodulatory drug (IMiD), and an anti-CD38 antibody in any order during the course of treatment.

Intensive dosing (e.g., q1wk for the first two 28-day cycles, then q2wk in subsequent 28-day cycles) or the combination therapy with dexamethasone can be administered to certain patients. In some embodiments, these patients must not have other therapeutic options known to provide clinical benefit in MM available. In some embodiments, these patients must have received at least 3 prior lines of anti-myeloma therapy and must be refractory to at least 1 agent in each of the following classes: PI, IMiD, and an anti-CD38 antibody. When the combination therapy with dexamethasone is administered to the patient, the antibody or antigen-binding fragment thereof as described herein can be administered under either the standard dosing schedule or the intensive dosing schedule.

In some embodiments, the combination therapy with dexamethasone and an IMiD can be administered to certain patients. In some embodiments, these patients must have received at least 2 prior lines of antimyeloma therapy, including at least 2 consecutive cycles of lenalidomide and a proteosome inhibitor (given separately or in combination), and must have documented IMWG disease progression on or within 60 days of completion of their last treatment. Patients with a history of autologous SCT (stem-cell transplantation) are eligible if the date of transplant was at least 12 weeks prior to initiation of SEA-BCMA treatment.

### Assays

The physical conditions of the subject treated by the methods described herein can be measured by any suitable assays known in the art. Non-limiting assays include immunohistochemical assays, radio imaging assays, in-vivo imaging, positron emission tomography (PET), single photon emission computer tomography (SPECT), magnetic resonance imaging (MRI), Ultra Sound, Optical Imaging, Computer Tomography, radioimmunoassay (RIA), ELISA (enzyme-linked immunosorbent assay), slot blot, competitive binding assays, fluorimetric imaging assays, Western blot, FACS, and the like.

In some embodiments, a biological sample is collected from the subject for an assay. The biological samples include, but are not limited to blood, serum, urine, plasma, the external secretions of the respiratory, intestinal, and genitourinary tracts, cerebrospinal fluid, peritoneal fluid, pleural fluid, cyst fluid, broncho alveolar lavage, lavage of any other part of the body or system in the body, and samples of any organ including isolated cells or tissues, where the cell or tissue can be obtained from an organ selected from, but not limited to lung, colon, kidney, pancreas, ovary, prostate, liver, skin, bone marrow, lymph node, breast, and/or blood tissue; stool or a tissue sample, or any combination thereof. Prior to performance of the assay, the sample can optionally be diluted with a suitable diluent. In some embodiments, cells obtained from the sample are cultured in vitro prior to performing the assay.

In some embodiments, the steady-state concentration of the anti-BCMA antibody in the serum of the subject can be measured.

One exemplary in vitro cell binding capacity assay to estimate the free anti-BCMA antibody in patients serum Involves pelleting a suspension of cultured MM1R cells and then re-suspending the pellet in serum from peripheral blood of subjects collected at different time points in treatment. After incubation at room temperature for 0.5 hour, the cells are washed and stained with a saturating amount of one of the anti-BCMA antibodies described herein conjugated to a fluorescent dye. After incubation at 4° C. in the dark for 0.5 hr, the cells are washed and fixed. Stained cells are analyzed on an Invitrogen Attune NxT flow cytometer. FlowJo V10 software is used to gate on viable cells and record the median fluorescent intensity (MFI). GraphPad Prism 8 is used for analysis.

One exemplary method of determining BCMA expression and binding by its ligands and an anti-BCMA antibody as described herein involves collecting bone marrow aspirates from a subject at baseline and after or during treatment, and then testing the samples by flow cytometry within one day of collection. MM cell detection can be performed using extracellular biomarker staining, for example, CD138, CD38, CD45, CD56, and CD28 staining and intracellular kappa and lambda light chains staining. Profiling of BCMA expression can be performed using, for example, two anti-BCMA antibodies: BCMA available for binding to anti-BCMA antibodies is detected using labeled anti-BCMA antibodies that bind BCMA in a competitive manner with a reference anti-BCMA antibody (e.g., one of the antibodies or antigen-binding fragments described herein such as the SEA-BCMA antibody described in the examples) and BCMA ligands (APRIL, etc.), while total extracellular BCMA is detected using a differently labeled anti-BCMA antibody that binds BCMA without competing with the reference antibody and BCMA ligands. Detection of APRIL, bound to BCMA on the MM cell surface, can also be performed. Each sample is split into 3 aliquots: one aliquot stained using only the MM gating antigens but no anti-BCMA or anti-APRIL antibodies (gating control), one aliquot stained with MM gating antigens and both labeled anti-BCMA antibodies, and one incubated for, for example, 2 hours at 37° C. with spiked BCMA (e.g., 100 μg/mL of spiked BCMA) before staining with MM gating antigens, APRIL, and the labeled anti-BCMA antibody detecting total extracellular BCMA. After staining, the cells are washed and fixed in 2% paraformaldyde, and the cells are analyzed on a flow cytometer.

### Kits

Also provided herein are kits that include: (a) one or more doses (e.g., 2, 3, 4, 5, 6, 7, 8, 9, 10, 11, 12, 13, 14, 15, 16, 17, 18, 19, 20, 21, 22, 23, 24, 25, or 26 doses) of a pharmaceutical composition (e.g., any of the pharmaceutical compositions described herein) comprising any of the antibodies or antigen-binding fragments thereof described herein, and (b) instructions or directions for performing any one of the methods described herein.

In some embodiments, the one or more doses can be provided in an injection device (e.g., a preloaded injection device). In some embodiments, the one or more doses can be provided as a lyophilized solid composition that can be reconstituted using a pharmaceutically acceptable buffer or solution (e.g., saline or phosphate buffered saline). In some embodiments, the one or more doses can be provided as a liquid composition (e.g., a liquid composition that can be administered to the subject via intravenous administration).

### EXAMPLES

### Example 1. Clinical Study of SEA-BCMA in Treatment of Multiple Myeloma

SEA-BCMA is a non-fucosylated monoclonal anti-BCMA antibody having the heavy chain amino acid sequence of SEQ ID NO: 13, and the light chain amino acid sequence of SEQ ID NO: 15.

Heavy Chain of SEA-BCMA
(SEQ ID NO: 13)
QVQLVQSGAEVKKPGASVKLSCKASGYTFTDYYIHWVRQAPGQG
LEWIGYINPNSGYTNYAQKFQGRATMTADKSINTAYVELSRLRS
DDTAVYFCTRYMWERVTGFFDFWGQGTMVTVSSASTKGPSVFPL
APSSKSTSGGTAALGCLVKDYFPEPVTVSWNSGALTSGVHTFPA
VLQSSGLYSLSSVVTVPSSSLGTQTYICNVNHKPSNTKVDKKVE
PKSCDKTHTCPPCPAPELLGGPSVFLFPPKPKDTLMISRTPEVT
CVVVDVSHEDPEVKFNWYVDGVEVHNAKTKPREEQYNSTYRVVS
VLTVLHQDWLNGKEYKCKVSNKALPAPIEKTISKAKGQPREPQV
YTLPPSRDELTKNQVSLTCLVKGFYPSDIAVEWESNGQPENNYK
TTPPVLDSDGSFFLYSKLTVDKSRWQQGNVFSCSVMHEALHNHY
TQKSLSLSPGK
Light Chain of SEA-BCMA
(SEQ ID NO: 15)
DIQMTQSPSSVSASVGDRVTITCLASEDISDDLAWYQQKPGKAP
KVLVYTTSSLQSGVPSRFSGSGSGTDFTLTISSLQPEDFATYFC
QQTYKFPPTFGGGTKVEIKRTVAAPSVFIFPPSDEQLKSGTASV
VCLLNNFYPREAKVQWKVDNALQSGNSQESVTEQDSKDSTYSLS
STLTLSKADYEKHKVYACEVTHQGLSSPVTKSFNRGEC

SEA-BCMA comprises a heavy chain variable region comprising a CDR1 comprising DYYIH (SEQ ID NO: 1), a CDR2 comprising YINPNSGYTNYAQKFQG (SEQ ID NO: 2), and a CDR3 comprising YMWERVTGFFDF (SEQ ID NO: 3), and a light chain variable region comprising a CDR1 comprising LASEDISDDLA (SEQ ID NO: 5), a CDR2 comprising TTSSLQS (SEQ ID NO: 6), and a CDR3 comprising QQTYKFPPT (SEQ ID NO: 7). SEA-BCMA comprises a heavy chain variable region comprising SEQ ID NO: 4, and a light chain variable region comprising SEQ ID NO: 8.

A clinical study to evaluate SEA-BCMA in a patient population whose disease has relapsed or is refractory to standard therapies, and for whom there remains no treatment options available, is ongoing and the initial data indicate that methods of treating multiple myeloma described herein provide a clinical benefit.

The immunospecificity and antitumor activity of SEA-BCMA have been demonstrated both in vitro and in vivo in BCMA-expressing MM models.

This study evaluated the safety and antitumor activity of SEA-BCMA in patients with RRMM. Specific objectives and corresponding endpoints for the study are summarized below (Table 2).

TABLE 2
Objectives and corresponding endpoints

| Primary Objectives | Corresponding Primary Endpoint |
|---|---|
| Evaluate the safety and tolerability of | Type, incidence, severity, seriousness, and |
| SEA-BCMA monotherapy in patients with | relatedness of adverse events (AEs) |
| relapsed or refractory multiple myeloma (RRMM) | Type, incidence, and severity of laboratory |
| Identify the maximum tolerated dose (MTD) | abnormalities |
| and/or optimal dose and schedule of SEA-BCMA | Incidence of dose-limiting toxicities (DLTs) |
| monotherapy in patients with RRMM | Type, incidence, severity, seriousness, and |
| Evaluate the safety and tolerability of SEA- | relatedness of adverse events (AEs) |
| BCMA in combination with dexamethasone in | Type, incidence, and severity of laboratory |
| patients with RRMM | abnormalities |
| Secondary Objectives | Corresponding Secondary Endpoints |
| Identify a recommended single-agent dose and | Incidence of DLTs, cumulative safety and |
| schedule of SEA-BCMA | activity by dose level |
| Assess the pharmacokinetics (PK) of SEA-BCMA | Maximum serum concentration and area under |
| Assess the immunogenicity of SEA-BCMA | the serum concentration-time curve |
| Assess the antitumor activity of SEA-BCMA | Incidence of SEA-BCMA antitherapeutic |
| | antibodies (ATA) |
| | Best response per the International Myeloma |
| | Working Group (IMWG) uniform response |
| | criteria (Kumar 2016) |
| | Objective response rate (ORR) |
| | Duration of objective response (OR) and |
| | complete response (CR) |
| | Progression-free survival (PFS) |
| | Overall survival (OS) |
| Exploratory Objectives | Corresponding Exploratory Endpoints |
| Assess incidence and level of BCMA expression | Characterization of BCMA expression on |
| in RRMM and relationship to clinical response to | malignant plasma cells |
| SEA-BCMA | Exploratory biomarkers of SEA-BCMA- |
| Assess the pharmacodynamic effects and | mediated pharmacodynamic effects |
| biomarkers of response, toxicity, and resistance to | Rate of MRD clearance |
| SEA-BCMA | Descriptive outcomes of qualitative interviews |
| Assess minimal residual disease (MRD) in | |
| patients with very good partial response (VGPR) | |
| or better | |
| Assess impact of SEA-BCMA in combination | |
| with dexamethasone on health-related quality of | |
| life (HRQoL) from the patient's perspective | |

### Summary of Study Design

### Monotherapy Dose-Escalation Cohort

The monotherapy dose-escalation portion of the trial was conducted in approximately 25 patients.

Enrollment in this study occurred on a cohort-by-cohort basis. Multiple cohorts were treated at each dose level, with a maximum of 4 patients treated per cohort. Decisions on dose escalation and subsequent cohort size were made in consultation with the safety monitoring committee (SMC) after completion of each cohort. Patients in the current cohort were observed for the full duration of the DLT period before the next cohort of patients was enrolled. In addition, as a precaution, for the first 2 patients in the study there was a 72-hour observation period before the next patient can be dosed. At dose levels above Dose Level 1, a 24-hour observation period was required after the first patient received their first dose of SEA-BCMA, prior to dosing subsequent patients at that dose level. At least 2 DLT-evaluable (DE) patients were treated per dose level until the first DLT was observed, then a minimum of 3 DE patients per dose level was required before escalation to all higher doses. Patients who were considered not evaluable for DLT during Cycle 1 were replaced. A minimum of 6 DE patients were observed at the estimated MTD before the MTD or optimal dose was determined. The MTD or optimal dose was estimated based on data from all patients across all evaluated doses.

De-escalation to a lower dose level could be performed at any time in consultation with the SMC. Intrapatient dose escalation to a dose level shown to be safe could be permitted in the event that a patient tolerates SEA-BCMA and achieves stable disease (SD) or better.

Patients continued on treatment until progressive disease or unacceptable toxicity, whichever occurred first.

SEA-BCMA was initially administered once every 2 weeks (q2wk) in 4-week cycles at the planned doses shown in Table 3; a dosing interval of every 4 weeks (q4wk) was explored.

TABLE 3
Dose escalation schema

| Dose Level | Dose (mg) |
|---|---|
| 1 | 100 |
| 2 | 200 |
| 3 | 400 |
| 4 | 800 |
| 5 | 1,600 |

### Monotherapy Expansion Cohort

To further characterize the safety and antitumor activity of SEA-BCMA, an expansion cohort of up to approximately 40 patients were enrolled. The dose and schedule for the expansion cohort were determined in consultation with the SMC based on the cumulative safety and activity demonstrated during dose escalation, which was completed without exceeding MTD at the doses tested.

### Monotherapy Intensive Dosing

The intensive dosing evaluates the safety and tolerability of SEA-BCMA dosed once a week (q1wk) during an induction phase (for 8 doses during the first 2 cycles of therapy); following the completion of the 8 week induction phase, patients who have not yet experienced confirmed disease progression proceeded to receive SEA-BCMA dosed q2wk during a maintenance phase (Cycle 3 and beyond, dosing at the recommended standard-schedule monotherapy expansion dose).

The intensive dosing includes a safety run-in at the recommended SEA BCMA monotherapy expansion dose (1600 mg), administered on the intensive dosing schedule (Day 1, Day 8, Day 15, and Day 22 of Cycles 1 and 2, and Day 1 and Day 15 of subsequent cycles). DLTs are being evaluated in the first 6 patients.

Patients who are deemed not evaluable for dose-limiting toxicity (DLT) during dose finding will be replaced for the determination of the dose of SEA-BCMA in combination with dexamethasone.

TABLE 4
Dose levels for monotherapy intensive dosing

| | Weekly Induction Dose, | Biweekly Maintenance Dose, |
|---|---|---|
| Dose Level | Cycles 1-2 (mg) | Cycles 3 and beyond (mg) |
| 1 | 1600 | 1600 |
| −1 (if Dose | 800 | 1600 |
| Level 1 is not | | |
| tolerated) | | |

### Dexamethasone Combination Therapy Cohorts

To characterize the safety and tolerability of SEA-BCMA in combination with dexamethasone, approximately 20 patients will be initially enrolled in each optional combination therapy cohort.

Enrollment into combination therapy cohorts will be initiated upon identification of tolerable SEA-BCMA monotherapy doses and schedules.

In Optional Cohort 1, SEA-BCMA will be administered on Day 1 and Day 15 of each 28-day cycle (standard dosing). Dexamethasone will be administered on Day 1, Day 8, Day 15, and Day 22 of each 28-day cycle.

In Optional Cohort 2, SEA-BCMA will be administered on Day 1, Day 8, Day 15, and Day 22 of Cycles 1 and 2, and Day 1 and Day 15 of subsequent cycles (intensive dosing). Dexamethasone will be administered on Day 1, Day 8, Day 15, and Day 22 of each 28-day cycle.

### Pomalidomide and Dexamethasone Combination Therapy Cohort

The Pomalidomide and Dexamethasone combination cohort will study the combination of SEA-BCMA with pomalidomide and dexamethasone in patients who have received at least 2 prior lines of antimyeloma therapy. SEA-BCMA will be administered on Day 1 and Day 15 of each 28 day cycle (standard dosing). Dexamethasone will be administered on Day 1, Day 8, Day 15, and Day 22 of each 28-day cycle. Pomalidomide will be administered on Days 1-21 of each 28-day cycle.

### Combination Therapy Cohorts Safety Run-In

The combination therapy cohorts will include a safety run-in at the recommended SEA BCMA monotherapy dose and schedule. DLTs will be evaluated in the first 6 patients enrolled in each combination therapy cohort.

Patients who are deemed not evaluable for DLT during dose finding will be replaced for the determination of the dose of SEA-BCMA in combination with dexamethasone.

### Dose-Limiting Toxicity (DLT)

The DLT-evaluation period was the first cycle of treatment. DLTs were graded according to the National Cancer Institute Common Terminology Criteria for Adverse Events (NCI-CTCAE), version 4.03, and defined as any of the following events during the DLT-evaluation period:
A delay of treatment by more than 7 days due to toxicity
Any adverse event (AE) ≥Grade 3, unless deemed by the SMC to be clearly unrelated to SEA-BCMA, except for the following AEs, which must meet these specified criteria to be considered a DLT:
   Grade 4 neutropenia lasting more than 5 days
   Thrombocytopenia ≥Grade 4, or Grade 3 thrombocytopenia with clinically significant bleeding
   Anemia ≥Grade 4 unrelated to underlying disease
   Any Grade ≥3 tumor lysis syndrome, including associated laboratory evaluations, that is not successfully managed clinically and that does not resolve within 7 days without end organ damage
   Any ≥Grade 4 infusion-related reactions (IRRs) or Grade 3 IRRs that do not resolve to ≤Grade 2 within 24 hours with infusion interruption, infusion rate reduction, and/or standard supportive measures. In the event of a Grade 3 IRR in ≥20% of patients (i.e., 2 or more in the first 10 patients), all subsequent patients will require premedication and/or modification of infusion approach per the recommendation of the SMC. For patients receiving premedication, any ≥Grade 3 IRR will be considered a DLT.
   Any Grade ≥3 asymptomatic laboratory abnormality that does not resolve, with or without intervention, to ≤Grade 1 or the baseline grade within 72 hours
   Any treatment-related death

### Stopping Criteria

The study was halted if any of the following occurred:
Rate of on-study toxic deaths unrelated to underlying disease occurring within 30 days of dose exceeded 10% (initially, 2 or more of the first 20 patients)
Rate of Grade 4 non-hematologic toxicity unrelated to underlying disease exceeded 25% (initially, 5 or more of the first 20 patients)
Rate of ≥Grade 4 allergic reactions that cannot be controlled with standard treatments exceeded 15% (initially, 3 or more of the first 20 patients)
Stopping criteria were continuously monitored throughout the study by the sponsor.

### Discussion and Rationale for Study Design

Initial clinical development of SEA-BCMA involved its evaluation in patients with RRMM that have no other therapeutic options known to provide clinical benefit available, and were candidates for SEA-BCMA treatment in the opinion of the treating physician. Prior therapies must include at least a proteasome inhibitor (PI), an immunomodulatory drug (IMiD), and an anti-CD38 antibody. Frontline and first relapse standard of care (SOC) treatments were expected to have failed in these patients prior to enrollment. Because BCMA is a broadly expressed tumor antigen in patients with MM, initial selection of patients based on BCMA expression was not required, although the relationship between target expression and outcome were explored in this phase 1 study.

The first portion of the study consisted of dose escalation in order to estimate the MTD and/or optimal dose of SEA-BCMA. Once dose escalation was complete and safety of the drug was demonstrated, an expansion cohort of approximately 40 patients were enrolled to further evaluate the safety and antitumor activity of SEA-BCMA at the standard q2wk dosing schedule. The expansion cohort allowed for the collection of additional information about the safety, tolerability, and activity of SEA-BCMA. This information was the basis for determining the recommended single-agent dose and schedule for SEA-BCMA. Because maintenance therapy had been shown to prolong remissions in patients with MM, patients were permitted to continue on treatment until progressive disease (PD) or unacceptable toxicity, which ever occurred first. In addition, intrapatient dose escalation to a dose level shown to be safe was permitted in the event that a patient tolerated SEA-BCMA and achieved a response of SD or better.

### Study Population

All patients met all of the enrollment criteria to be eligible for this study and prior to study drug administration (within 1 day of dosing) on Cycle 1 Day 1.

To be eligible for retreatment, all patients met inclusion and exclusion criteria outlined in the below sections.

### Inclusion Criteria

1. Diagnosis of multiple myeloma (MM) requiring systemic therapy as defined by International Myeloma Working Group (IMWG) 2014 criteria (Rajkumar et al., Lancet Oncol 15(12): e538-48, 2014). 2. Patients must have MM that is relapsed or refractory and must not have other therapeutic options known to provide clinical benefit in MM available, and be a candidate for SEA-BCMA treatment in the opinion of the treating physician.

(a) Patients that are enrolled in the dose escalation cohorts and does expansion cohorts must not have other therapeutic options known to provide clinical benefit in MM available. Patients' prior lines of therapy for patients enrolled in the dose escalation study must include at least a proteasome inhibitor (PI), an immunomodulatory drug (IMiD), and an anti-CD38 antibody in any order during the course of treatment. Participants who could not tolerate a PI, IMiD, or anti-CD38 antibody are allowed.

(b) Patients enrolled in the monotherapy intensive dosing or dexamethasone combination therapy must not have other therapeutic options known to provide clinical benefit in MM available. Patients must have received at least 3 prior lines of antimyeloma therapy and must be refractory to at least 1 agent in each of the following classes: PI, IMiD, and an anti-CD38 antibody.

(c) Patients enrolled in the pomalidomide and dexamethasone combination therapy must have received at least 2 prior lines of antimyeloma therapy, including at least 2 consecutive cycles of lenalidomide and a proteosome inhibitor (given separately or in combination), and must have documented IMWG disease progression on or within 60 days of completion of their last treatment. Patients with a history of autologous SCT are eligible if the date of transplant was at least 12 weeks prior to initiation of SEA-BCMA treatment.

Measurable disease, as defined by one or more of the following:

a. Serum monoclonal paraprotein (M-protein) level ≥0.5 g/dL; for IgA or IgD myeloma patients, serum IgA or serum IgD ≥0.5 g/dL is acceptable.

b. Urine M-protein level ≥200 mg/24 hr

c. Serum immunoglobulin free light chain ≥10 mg/dL and abnormal serum immunoglobulin kappa lambda free light chain ratio

Age 18 years or older.

An Eastern Cooperative Oncology Group (ECOG) Performance Status score of 0 or 1 (e.g., conversion of performance status using Karnofsky and Lansky scales, if applicable).

Life-expectancy of >3 months in the opinion of the investigator

The following baseline laboratory data (hematologic criteria must be met in the absence of growth factor or platelet transfusion support):

a. Estimated glomerular filtration rate (eGFR) ≥30 mL/min/1.73 m² per the Modified Diet in Renal Disease (MDRD) equation

b. Absolute neutrophil count (ANC) ≥1000/μL

c. Platelet count ≥75,000/μL

Patients of childbearing potential, under the following conditions:

a. Must have a negative serum or urine pregnancy test (minimum sensitivity 25 mIU/mL or equivalent units of beta human chorionic gonadotropin [β-hCG]) result within 7 days prior to the first dose of SEA-BCMA. Patients with false positive results and documented verification that the patient is not pregnant are eligible for participation.

b. Must agree not to try to become pregnant during the study and for at least 6 months after the final dose of SEA-BCMA.

c. Must agree not to breastfeed or donate ova, starting at time of informed consent and continuing through 6 months after the final dose of SEA-BCMA.

d. If sexually active in a way that could lead to pregnancy, must consistently use 2 highly effective methods of birth control starting at time of informed consent and continuing throughout the study and for at least 6 months after the final dose of study drug.

Patients who can father children, under the following conditions:

a. Must agree not to donate sperm starting at time of informed consent and continuing throughout the study period and for at least 6 months after the final dose of study drug administration.

b. If sexually active with a person of childbearing potential in a way that could lead to pregnancy, must consistently use 2 highly effective methods of birth control starting at time of informed consent and continuing throughout the study and for at least 6 months after the final dose of study drug administration.

c. If sexually active with a person who is pregnant or breastfeeding, must consistently use one of 2 contraception options starting at time of informed consent and continuing throughout the study and for at least 6 months after the final dose of SEA-BCMA administration.

Subjects of childbearing potential and are enrolled in the combination therapy that involve pomalidomide, the patients must meet the following conditions:
a. Must have 2 negative serum or urine pregnancy tests (minimum sensitivity 25 mIU/mL or equivalent units of HCG). One 10-14 days prior to start of the study drug and one 24 hours prior to the start of study drug
b. Must agree not to try to become pregnant during the study and for at least 6 months after the final dose of study drug administration
c. Must agree not to breastfeed or donate ova, starting at time of informed consent and continuing through 6 months after the final dose of study drug. Must agree to not donate blood for at least 90 days following completion of study treatment
d. If sexually active in a way that could lead to pregnancy, must consistently use 2 highly effective methods of birth control for 4 weeks prior to the start of treatment with study drugs and continuing throughout the study and for at least 6 months after the final dose of study drug.

Subjects who can father children and are enrolled in the combination therapy that involve pomalidomide, the patients must meet the following conditions:
a. Must agree not to donate sperm starting at time of informed consent and continuing throughout the study period and for at least 6 months after the final dose of study drug. Must agree to not donate blood for at least 90 days following completion of study treatment.
b. If sexually active with a person of childbearing potential in a way that could lead to pregnancy, or a person who is pregnant or breastfeeding, must consistently use 2 highly effective methods of birth control, one of which must be a latex or synthetic condom, starting at time of informed consent and continuing throughout the study and for at least 6 months after the final dose of study drug.

In addition, subjects are enrolled in the combination therapy that involve pomalidomide must be willing and able to comply with the Pomalyst® Risk Evaluation and Mitigation Strategy (REMS) program.

Furthermore, the patient must provide written informed consent.

### Exclusion Criteria

Prior exposure to any other BCMA-directed therapy.

History of another malignancy within 3 years before the first dose of SEA-BCMA, or any evidence of residual disease from a previously diagnosed malignancy. Exceptions are malignancies with a negligible risk of metastasis or death (e.g., 5-year overall survival ≥90%), such as adequately treated carcinoma in situ of the cervix, non-melanoma skin carcinoma, localized prostate cancer, ductal carcinoma in situ, or Stage I uterine cancer.

Active cerebral/meningeal disease related to the underlying malignancy. Patients with a history of cerebral/meningeal disease related to the underlying malignancy are allowed if prior central nervous system disease has been treated.

Any uncontrolled Grade 3 or higher (per the NCICTCAE, Version 4.03) viral, bacterial, or fungal infection within 2 weeks prior to the first dose of SEA-BCMA. Routine antimicrobial prophylaxis is permitted.

Positive for hepatitis B by surface antigen expression. Active hepatitis C infection (positive by polymerase chain reaction or on antiviral therapy for hepatitis C within the last 6 months). Patients who have been treated for hepatitis C infection are permitted if they have documented sustained virologic response of 12 weeks.

Known to be positive for human immunodeficiency virus (HIV).

Patients with previous allogeneic stem cell transplant (SCT).

Documented history of a cerebral vascular event (stroke or transient ischemic attack), unstable angina, myocardial infarction, or cardiac symptoms consistent with congestive heart failure, Class III-IV, New York Heart Association (see Appendix F) within 6 months prior to their first dose of SEA-BCMA. For combination therapy with pomalidomide: unable to tolerate thromboembolic prophylaxis while on study.

Current therapy with other systemic anti-neoplastic or investigational agents.

Chemotherapy, radiotherapy, biologics, investigational agents, and/or other antitumor treatment with immunotherapy that is not completed 4 weeks prior to first dose of SEA-BCMA, or 2 weeks if progressing and recovered from clinically significant toxicity associated with the treatment. CAR T-cell therapy that is not completed 8 weeks prior to first dose of SEA-BCMA. Palliative radiotherapy to a single site of disease is allowed with the approval of the medical monitor.

Systemic treatment with either corticosteroids (>10 mg daily prednisone equivalent) or other immunosuppressive medications within 14 days of enrollment. Inhaled or topical steroids and adrenal replacement steroid doses ≤10 mg daily prednisone equivalent are permitted.

Patients who are breastfeeding, pregnant, or planning to become pregnant from time of informed consent until 6 months after final dose of study drug administration.

Known hypersensitivity to any excipient contained in the drug formulation of SEA-BCMA.

Patients with plasma cell leukemia (>2.0×10⁹/L circulating plasma cells by standard differential), Waldenström's macroglobulinemia, POEMS syndrome (polyneuropathy, organomegaly, endocrinopathy, monoclonal protein, and skin changes), or clinically significant amyloidosis.

Moderate or severe hepatic impairment, as indicated by any of the following:

a. Serum total bilirubin >1.5× upper limit of normal (ULN). For patients with Gilbert's disease, total bilirubin >3×ULN.

b. Alanine aminotransferase (ALT) or aspartate aminotransferase (AST) >3×ULN Significant comorbid condition or disease which in the judgment of the investigator would place the subject at undue risk or interfere with the proper assessment of safety and toxicity of the SEA-BCMA.

For combination therapy only: known intolerance to corticosteroids.

For combination therapy only: any uncontrolled psychoses.

For combination therapy with pomalidomide: Prior treatment with pomalidomide, or history of hypersensitivity reaction of prior IMiD therapy (thalidomide or lenalidomide).

For combination therapy only: Grade ≥2 peripheral neuropathy.

For combination therapy only: Gastrointestinal disease that may significantly alter the absorption of pomalidomide.

### Discontinuation of Study Treatment

A patient's study treatment may be discontinued for any of the following reasons:

Progressive disease (PD)

AE

Pregnancy

Investigator decision

Patient decision, non-AE

Study termination by sponsor

Other, non-AE

In monotherapy, patients who discontinued SEA-BCMA were considered discontinued from study treatment. Patients who discontinue from study treatment will remain on study for follow-up until withdrawal of consent, death, or study closure, whichever occurs first.

In combination therapy, patients who discontinued SEA BCMA and dexamethasone will be considered discontinued from study treatment. Patients receiving dexamethasone who discontinued corticosteroid therapy may continue to receive SEA-BCMA as monotherapy with medical monitor approval. Patients who discontinued SEA-BCMA will be considered discontinued from study treatment.

### Patient Withdrawal From Study

Any patient may be discontinued from the study for any of the following reasons:
a) Patient withdrawal of consent;
b) Retreatment;
c) Study termination by sponsor;
d) Lost to follow-up;
e) Death;
f) Other.

### Treatments

SEA-BCMA is a non-fucosylated monoclonal antibody directed against BCMA.

Guidance for intrapatient dose-escalation for patients who have the potential to achieve greater benefit at a dose higher than the dose-level assigned during dose-escalation is described herein.

### Description

SEA-BCMA is a sterile, preservative-free, colorless to light yellow, clear to slightly opalescent solution with no visible particulate matter. SEA-BCMA was supplied in single-dose glass vials. The drug product solution was diluted in sterile 0.9% sodium chloride injection, United States Pharmacopeia (USP), or equivalent, for intravenous (IV) administration.

SEA-BCMA drug product was labeled with a nominal content of 100 mg/vial. Each vial contained 110 mg of SEA-BCMA, which allowed the label quantity to be withdrawn for use. SEA-BCMA drug product consists of SEA-BCMA (20 mg/mL), histidine, arginine, trehalose, and polysorbate 80. The pH of the product was approximately 6.5.

### Dose and Administration

SEA-BCMA was administered at the assigned dose by IV infusion. SEA-BCMA was not administered as an IV push or bolus. SEA-BCMA was not mixed with other medications.

On Cycle 1, Day 1, patients were closely observed in the clinic for at least 6 hours after completion of study treatment administration during dose escalation. Vital signs were collected. Additional monitoring for subsequent cycles was considered upon review of safety data. The observation period after completion of study treatment administration on Cycle 1, Day 1 was reduced to 2 hours during monotherapy dose expansion and in monotherapy intensive dosing and combination therapy cohorts following review of data from the dose escalation cohort, in which there were no instances of delayed-onset infusion-related reactions (IRRs).

Infusion duration varied depending on the method of infusion administration and the SEA-BCMA dose.

The initial approach to SEA-BCMA administration was stepwise infusion. In a stepwise infusion, the infusion rate was increased at set time intervals until a defined maximum rate of infusion was reached. The first infusion of SEA-BCMA was initiated at a rate of 50 mg/hour. If the first 30 minutes was well-tolerated, the rate was incrementally increased (no greater than 2-fold increase in rate) every 30 minutes as tolerated until a maximum rate (400 mg/hour) was reached. With subsequent infusions, the infusion rate could be increased more rapidly in shorter time intervals; e.g., after the first 15 minutes, the rate could be incrementally increased (no greater than 2-fold increase in rate) every 15 minutes as tolerated until the maximum rate was reached.

As clinical experience with stepwise infusions evolves, the maximum rate may be increased or decreased based on accumulating safety data and/or recommendations of the SMC. In addition, alternative approaches to SEA-BCMA administration may be evaluated to manage potential safety signals, including IRRs, as recommended by the SMC. These may include systematic implementation of the following strategies: extending the planned infusion duration, fixed-duration infusion (administration at a fixed infusion rate), divided-dose administration, or a change in premedications.

### Fixed-Duration Infusion

Some criteria were considered regarding fixed-duration infusion: If fixed-duration infusion is implemented, the SEA-BCMA infusion duration is defined by the physician. As clinical experience with SEA-BCMA infusion evolves, the infusion duration may be increased or decreased based on accumulating safety data and/or recommendations of the SMC.

In an individual patient, if the patient is unable to tolerate the infusion, the infusion duration may be increased; the infusion duration in subsequent infusions may also be increased per investigator discretion with medical monitor approval. Conversely, if a patient does not experience an IRR greater than Grade 1 with consecutive infusions, the infusion duration may be shortened (i.e., administered at a faster rate) at the discretion of the investigator with medical monitor approval, the implementation of which may be dose-cohort specific.

If a fixed infusion rate is implemented, the dose is administered at a fixed rate rather than over a fixed time.

For example, for a fixed infusion rate of 50 mg/hour, a dose of 100 mg would be infused over 2 hours. As clinical experience with administration at a fixed infusion rate evolves, the rate may be increased, or decreased, based on accumulating safety data and/or recommendations of the SMC.

In an individual patient, if the patient is unable to tolerate the infusion rate, the infusion rate may be decreased in subsequent infusions per investigator discretion with medical monitor approval. Conversely, if an individual patient does not experience an IRR greater than Grade 1 with consecutive infusions, the infusion rate may be increased at the discretion of the investigator with medical monitor approval.

### Divided-Dose Administration

Some criteria were considered regarding divided-dose administration:

If divided-dose administration is implemented, the dose is divided and administered separately within a time period. For example, the dose could be divided in 2 parts, in which the first 10% of the dose is infused over approximately 45 minutes, followed by a 30-minute observation period as the patient remains in the infusion chair. If the investigator determines that the patient has tolerated the initial SEA-BCMA infusion, the remaining 90% is infused over approximately 45 minutes.

### Dose Modifications

On a per-patient basis, lengthening of dosing intervals for toxicity, including DLT, were allowed upon approval by the medical monitor. Patients who experienced DLT in Cycle 1 did not receive further treatment with SEA-BCMA, unless clinical benefit is demonstrated with adequately managed toxicity and there was approval from the medical monitor. Examples of clinical benefit include an objective response (OR) assessed by imaging, laboratory assessment, or physical examination; or SD and clinical improvement in disease-related symptoms per investigator. If clinical benefit was demonstrated, the dosing interval was lengthened by 50%-100% after discussion with the medical monitor. The type and severity of the AE observed were taken into consideration to inform the decision. For patients treated at the lowest dose level, the dosing interval may be lengthened, or the patient may be discontinued from treatment.

If a patient had a clinically significant, unresolved AE on the planned dosing day, the dose was delayed for up to 7 days. Dosing delays due to other reasons or lasting >7 days were discussed with the medical monitor; during the DLT period, patients did not receive further treatment with SEA-BCMA unless clinical benefit was demonstrated with adequately managed toxicity and there was approval from the medical monitor. For patients requiring a dose delay >7 days due to an unresolved AE, subsequent doses were reduced or the dosing interval were lengthened by 50-100% after discussion with the medical monitor. Dose delays extending longer than twice the length of the dosing interval required patient discontinuation from study treatment.

In once every 2 weeks (q2wk) dosing, if a patient had a clinically significant, unresolved AE on Day 15 that prevented dosing, the Day 15 visit would be delayed for 7 days. On the seventh day, if a patient could not receive the dose, the second dose of the cycle would be eliminated, the Day 15 visit will be skipped, and the Day 22 visit will be performed. If the Day 15 dose was delayed for 7 days, study assessments required for Day 15-28 would be delayed by the same number of days as the dose delay, and study drug administration for the next cycle would be delayed by at least the same number of days.

In intensive dosing weekly induction Cycles 1 and 2, if a patient has a clinically significant, unresolved AE that prevents dosing on Day 8, 15, or 22, the dose may be delayed for ≤3 days. On the third day, if a patient can not receive the dose, the dose of SEA-BCMA will be eliminated and the corresponding visit will be skipped; dosing and visit schedule will resume the following week (e.g. at Day 22, if Day 15 was skipped). However, if a Day 8, 15, or 22 dose is delayed for ≤3 days, subsequent study assessments within the same cycle will be delayed by the same number of days as the dose delay, and study drug administration for the next dose will be delayed by at least the same number of days.

During the DLT period (Cycle 1), growth factor and transfusion support was discouraged unless medically indicated; patients who receive growth factor (e.g., G-CSF or GM-CSF) or transfusion support (other than red blood cell transfusions for MM-related anemia) during this period for reasons other than DLT may not be evaluable for DLT. Consideration were given for growth factor support for prophylaxis or treatment of cytopenias in subsequent cycles (Table 5). During dose escalation, patients with Grade 4 neutropenia had a follow up complete blood count (CBC) with differential obtained 5 days from the time of assessment for evaluation of DLT. In addition, patients with Grade 3 electrolyte abnormalities had a follow up chemistry panel obtained 72 hours from the time of assessment for evaluation of DLT. Serum chemistry and complete blood counts (CBCs) were collected minimally on a weekly schedule during dose delays resulting from toxicity.

Table 5 describes the recommended dose modifications for study treatment-associated toxicity.

TABLE 5
Recommended dose modifications for SEA-BCMA-associated toxicity

| Toxicity | Grade 1 | Grade 2 | Grade 3 | Grade 4 |
|---|---|---|---|---|
| Non-hematologic | Continue at | Continue at | Withhold dose until | Discontinue study |
| (AE or laboratory | same dose | same dose | toxicity is ≤ Grade 1 | treatment |
| abnormality) | level | level | or baseline^{a}, and then | |
| | | | resume treatment at | |
| | | | the same dose level | |

| | | | |
|---|---|---|---|
| Hematologic | Continue at | Continue at | First occurrence: Withhold dose until |
| (neutropenia, | same dose | same dose | resolution to ≤ Grade 2 or baseline; for Grade 3 |
| thrombocytopenia, | level | level | events, resume treatment at the same dose |
| and anemia) | | | level; for Grade 4 events, either resume |
| | | | treatment at the same dose level after |
| | | | discussion with the medical monitor or |
| | | | discontinue treatment at the discretion of the |
| | | | investigator. Treatment delay of up to 7 days is |
| | | | permitted.^{a} |
| | | | Second occurrence: Withhold dose until |
| | | | toxicity is ≤ Grade 2 or baseline^{a}. Either |
| | | | resume treatment at the same dose level with |
| | | | growth factor support after discussion with the |
| | | | medical monitor or discontinue study treatment |
| | | | at the discretion of the investigator^{b} |

| | |
|---|---|
| Infusion-related | See Section I.A.1 |
| reaction | |

^{a}Treatment delays of >7 days are to be discussed with the medical monitor

Intrapatient dose escalation were permitted in the event that a patient tolerates at least 1 cycle of SEA-BCMA and achieves SD or better. Additional treatment cycles may be administered at 1 dose level below the currently enrolling dose level for dose escalation (or at the MTD if it has been determined).

### Dexamethasone

Dexamethasone will be given on Days 1, 8, 15, and 22 of each 28-day cycle. Dexamethasone will be administered as an IV infusion or orally (PO) at a dose of 40 mg. The dose of dexamethasone is 20 mg for patients ≥75 years, or with BMI <18.5, or known to be intolerant of dexamethasone 40 mg. On days when SEA-BCMA is administered, dexamethasone is administered 1 to 3 hours prior to the SEA-BCMA infusion.

### Dose Modifications

Dose modifications and supportive care by toxicity are listed in Table 6.

TABLE 6
Dose modifications for dexamethasone-associated toxicity

| | | Recommended Dose |
|---|---|---|
| CTCAE Category | Toxicity | Modification/Supportive Care |
| Gastrointestinal | Grade 1-2 dyspepsia, gastric | Treat with a proton pump inhibitor such as |
| | or duodenal ulcer, gastritis | omeprazole. |
| | requiring medical | If symptoms persist, decrease |
| | management | dexamethasone dose by 50% |
| | ≥Grade 3 requiring | Hold dexamethasone until symptoms are |
| | hospitalization or surgery | adequately controlled. Then, restart at 50% |
| | | of current dexamethasone dose along with |
| | | concurrent therapy with a proton pump |
| | | inhibitor such as omeprazole. If symptoms |
| | | persist despite above measure, discontinue |
| | | dexamethasone and do not resume. |
| | Acute pancreatitis | Discontinue dexamethasone and do not |
| | | resume. |
| Cardiovascular | ≥Grade 3 edema limiting | Diuretics as needed and decrease |
| | function and unresponsive to | dexamethasone dose by 25%; if edema |
| | therapy or anasarca | persists despite above measures, decrease |
| | | dose to 50% of initial dose; discontinue |
| | | dexamethasone and do not resume if |
| | | symptoms persist despite 50% reduction |
| Neurology/Psychiatric | ≥Grade 2 confusion or mood | Hold dexamethasone until symptoms |
| | alteration interfering with | adequately controlled. Restart at 50% of |
| | function | current dose. If symptoms persist despite |
| | | above measure, discontinue dexamethasone |
| | | and do not resume |
| Musculoskeletal | ≥Grade 2 muscle weakness, | Decrease dexamethasone dose by 25%; if |
| | symptomatic and interfering | weakness persists despite above measures, |
| | with function but not | decrease dose to 50% of initial dose; |
| | interfering with activities of | discontinue dexamethasone and do not |
| | daily living | resume if symptoms persist despite 50% |
| | >Grade 2 muscle weakness, | Hold dexamethasone until muscle weakness |
| | symptomatic and interfering | is ≤ Grade 1 or baseline. Then decrease |
| | with activities of daily living | dexamethasone dose by 25% and resume; if |
| | | weakness persists despite above measures, |
| | | decrease dose to 50% of initial dose; |
| | | discontinue dexamethasone and do not |
| | | resume if symptoms persist despite 50% |
| Metabolic^{a} | ≥Grade 3 hyperglycemia | Treatment with insulin or oral |
| | | hypoglycemic agents as needed. If |
| | | uncontrolled despite above measure, |
| | | decrease dose by 25% decrements until |
| | | levels are satisfactory |
| Constitutional | ≥Grade 2 insomnia | Decrease dexamethasone dose by 50% |

^{a}Patients who enter the study with elevated hemoglobin A1c (HbA1c) (≥6.5%) or fasting glucose (≥126 mg/dL) at screening must be referred to an appropriate provider for glucose management prior to or within 1 week of starting study treatment in Cycle 1.

### Pomalidomide

Pomalidomide can be given once daily at a dose of 4 mg PO on days 1-21 of each 28 day cycle. On Cycle 1 Day 1, pomalidomide can be administered 1 to 3 hours prior to SEA-BCMA infusion, to allow for appropriate timing of PK analysis. Pomalidomide can subsequently be taken at approximately the same time each day, and without food (at least 2 hours before or 2 hours after each meal). The capsules can be swallowed whole with water and not opened, broken, or chewed.

Suggested dose modifications and supportive care by toxicity are provided in the table below.

TABLE 7
Recommended dose modifications for pomalidomide-related toxicity

| NCI-CTCAE | | |
|---|---|---|
| Category | Toxicity | Recommended Dose Modification |
| Hematologic | Grade 4 | Withhold pomalidomide treatment and follow |
| | thrombocytopenia, each | complete blood count weekly until |
| | occurrence | thrombocytopenia improves to ≤ Grade 2, then |
| | | resume pomalidomide at 1 mg less than previous |
| | | dose PO daily*. |
| | Grade 4 neutropenia or | Withhold pomalidomide treatment and follow |
| | any grade febrile | complete blood count weekly until neutropenia |
| | neutropenia, each | improves to ≤ Grade 2, then resume pomalidomide |
| | occurrence | at 1 mg less than previous dose PO daily*. |
| Gastrointestinal | Grade 1 or higher AST or | Withhold pomalidomide treatment until liver |
| | ALT elevation, or | enzymes return to normal or baseline. Consider |
| | elevation from baseline | resuming pomalidomide at 1 mg less than previous |
| | CTCAE grade | dose^{a}. |
| Other | ≥Grade 3 nonhematologic | Withhold pomalidomide treatment and follow for |
| | toxicity | improvement of toxicity to ≤ Grade 2, then resume |
| | | pomalidomide at 1 mg less than previous dose PO |
| | | daily. |
| | | Permanently discontinue pomalidomide for |
| | | angioedema, anaphylaxis, Grade 4 rash, skin |
| | | exfoliation, bullae, or any other severe dermatologic |
| | | reaction. |

*If toxicities occur after dose reduction to 1 mg, discontinue pomalidomide.
ALT = alanine aminotransferase, AST = aspartate aminotransferase, CYP = cytochrome P, NCI-CTCAE = National Cancer Institute's Common Terminology Criteria for Adverse Events, PO = taken orally
If necessary to coadminister strong inhibitors of CYP1A2 in the presence of strong inhibitors of CYP3A4 and P-glycoprotein, consider reducing pomalidomide dose by 50%.
Dose modification guidance is based on pomalidomide prescribing information, which contains additional guidance on pomalidomide dosing.

### Management of Adverse Reaction

### 1. Management of SEA-BCMA Infusion Reactions

IRRs may occur during the infusion of monoclonal antibody therapies such as SEA-BCMA. The infusion should be administered at a site properly equipped and staffed to manage anaphylaxis should it occur. All supportive measures consistent with optimal patient care should be given throughout the study according to institutional standards. Supportive measures may include extending the infusion time and/or administering medications for IRRs.

During dose escalation, additional mitigation strategies may be explored to manage IRRs. These may be implemented upon SMC recommendation, and may include but are not limited to any or all of the following:

Slowing, interruption, or other adjustments in the administration of SEA-BCMA

Potential premedication or postmedication for infusions, for example:
Antihistamines, such as diphenhydramine 50 mg IV or equivalent and famotidine 40 mg IV or equivalent
Antipyretics, such as acetaminophen 500-1,000 mg PO
Antiemetics, such as ondansetron
IV fluid support, such as normal saline
Anti-rigor medication, such as meperidine
Vasopressors
Corticosteroids, such as hydrocortisone 100 mg IV or equivalent or methylprednisolone 40 mg IV or equivalent (for patients not receiving dexamethasone as combination therapy)

Recommendations for the management of IRRs are detailed in Table 8. IRRs should be graded according to NCI-CTCAE, version 4.03, guidelines.

TABLE 8
Management of infusion-related reactions
IRR Grade^{a}

| Grade 1 | Grade 2 | Grade 3 | Grade 4 |
|---|---|---|---|
| Mild transient | SEA-BCMA treatment | Prolonged (e.g., not | Life-threatening |
| reaction; | interruption indicated | rapidly | consequences; urgent |
| SEA-BCMA | but | responsive to symptomatic | intervention indicated |
| treatment | responds promptly to | medication and/or brief | |
| interruption not | symptomatic treatment | interruption of infusion); | |
| indicated; | (e.g., | recurrence of symptoms | |
| intervention not | antihistamines, | following initial | |
| indicated | NSAIDS, | improvement; | |
| | narcotics, IV fluids); | hospitalization indicated | |
| | prophylactic | for clinical sequelae | |
| | medications | | |
| | indicated for ≤24 hr | | |

Treatment Recommendations

| | | | |
|---|---|---|---|
| Monitor vital signs | Hold SEA-BCMA | Stop SEA-BCMA | Stop SEA-BCMA |
| more frequently | treatment. Monitor vital | treatment. Institute | treatment immediately. |
| until symptoms | signs more frequently | additional medical | Hospitalization. |
| have resolved and | until symptoms have | management as indicated. | |
| patient is medically | resolved and patient is | Consider hospitalization. | |
| stable. Administer | medically stable. | | |
| symptomatic | Administer | | |
| treatment as | symptomatic treatment | | |
| medically indicated. | as medically indicated. | | |
| | If patient responds | | |
| | promptly and is | | |
| | medically stable in the | | |
| | opinion of the | | |
| | investigator, | | |
| | SEA-BCMA treatment | | |
| | may be continued at a | | |
| | slower rate. | | |

Dose Modifications

| | | | |
|---|---|---|---|
| Consider | Consider premedication | Patients with an IRR that | Permanently |
| premedication with | with subsequent | resolves to baseline or | discontinue from study |
| subsequent | SEA-BCMA treatment. | Grade 1 or lower within | treatment. |
| SEA-BCMA | Consider slower | approximately 2 hours | |
| treatment. | infusion rate. If | after intervention may | |
| | recurrent after the | continue SEA BCMA at | |
| | above measures, | the same dose with | |
| | consider dose reduction | premedications required | |
| | to 1 dose level below | prior to all subsequent | |
| | current dose. | doses, if approved by the | |
| | | medical monitor. | |
| | | OR | |
| | | Permanently discontinue | |
| | | from study treatment. | |

Per NCI-CTCAE version 4.03

If anaphylaxis occurs, administration of SEA-BCMA should be immediately and permanently discontinued.

All Grade 3 or 4 events of IRR (with onset during infusion or within ≤24 hr after infusion) or hypersensitivity reaction (with onset occurring >24 hr after infusion) must be reported to the sponsor or designee immediately, regardless of relationship to SEA-BCMA. All Grade 4 events are serious adverse events (SAEs) and are to be reported within the SAE reporting timeframe of 24 hours.

Patients experiencing a ≥Grade 3 IRR or delayed hypersensitivity reaction must have an Infusion/Hypersensitivity Reaction (IHR) Visit and an IHR Follow-up Visit for evaluation and collection of blood samples for analysis of the mechanism of action of the reaction.

### Required Premedication and Postmedication for SEA-BCMA

Routine premedication for infusion reactions should not be administered prior to the first dose of SEA-BCMA. However, patients who experienced IRRs received subsequent treatment with premedication such as antihistamines (e.g., diphenhydramine 50 mg IV or equivalent and famotidine 40 mg IV or equivalent), corticosteroids (e.g., hydrocortisone 100 mg IV or equivalent), or acetaminophen (e.g., 500-1,000 mg PO) at least 30 minutes prior to the infusion. As clinical experience with SEA-BCMA infusions evolves, routine premedication prior to the first dose of study treatment may be instituted, as recommended by the SMC.

There were no required postmedications for SEA-BCMA.

In intensive dosing cohort, dexamethasone combination therapy cohort, and pomalidomide and dexamethasone combination therapy cohort, routine premedication for infusion reactions must be administered prior to SEA-BCMA infusion per the following regimen, unless contraindicated or recommended otherwise by the SMC or medical monitor: Antipyretic+Antihistamine: administer approximately 45 to 90 minutes prior to SEA BCMA infusion (required for all patients for all doses during Cycle 1 and Cycle 2)

(1) Acetaminophen, oral, 650 to 1000 mg

(2) Diphenhydramine, oral or IV, 25 to 50 mg (or equivalent H1 blocker)

If no IRR (infusion-related reactions) is experienced during Cycle 1 or Cycle 2: one or both premedications may be omitted starting with Cycle 3 Day 1 dose.

If IRR occurs despite acetaminophen+antihistamine

Treat with supportive care based on symptoms.

For monotherapy patients (not receiving Dexamethasone), add:

Methylprednisolone, IV, 100 mg (or equivalent dosage intermediate to long-acting corticosteroid) as required premed 1 to 3 hours prior to next SEA-BCMA infusion. If this infusion is tolerated without IRR, methylprednisolone dose may be reduced to 60 mg (or equivalent dosage of intermediate to long-acting corticosteroid), administered either oral or IV, prior to subsequent doses.

Additional premedications (e.g., H2 blockers or leukotriene inhibitors) may be considered.

For combination patients (receiving Dexamethasone), add:

H2 blocker (famotidine 40 mg IV or equivalent) as required premed 45 to 90 minutes prior to all subsequent SEA-BCMA doses

Additional premedications (e.g., leukotriene inhibitors) may be considered.

### Study Assessments

### Screening/Baseline Assessments

Only patients who met all inclusion and exclusion criteria were enrolled in this study. Assessments began after obtaining a signed informed consent from the patient.

Patient medical history includes a thorough review of significant past medical history, current conditions, any treatment for prior malignancies and response to prior treatment, and any concomitant medications. The number of prior lines of therapy were determined using the criteria established by Rajkumar et al. (Rajkumar et al., Blood 126(7): 921-2, 2015). In brief:
If a treatment regimen is discontinued for any reason and a different treatment regimen is started, it is considered a new line of therapy.
A new line of therapy is also considered to start when an unplanned substitution or addition of 1 or more drugs is made to an existing course of therapy for any reason.
In patients undergoing >1 ASCT (except in the case of a planned tandem ASCT), each transplant that follows the first one should be considered a new line of therapy.
A planned course of therapy that has multiple phases, such as induction therapy followed by the first ASCT and maintenance therapy, is considered to be a single line of therapy.

A baseline plasmacytoma scan was conducted during screening only in cases of suspected or known plasmacytoma. During treatment, plasmacytoma evaluations were performed at any time to confirm a response of PR or better, or as clinically indicated to confirm PD.

Bone marrow aspirate (including a bone marrow aspirate clot) and biopsy were required as part of the baseline visit.

Physical examinations included assessments of the following body parts/systems: abdomen, extremities, head, heart, lungs, neck, and neurological. Weight and height were also measured; measurements of height obtained within the prior 12 months may be utilized.

Blood and urine tests included CBC with differential, serum chemistry panel, serology (hepatitis B and C), PT/PTT/INR, hBA1c (for patients in the combination cohort) and urinalysis. A pregnancy test was conducted for patients of childbearing potential. Urinalysis with microscopy was required if urinalysis results were abnormal. Spot urine for UPC ratio calculation was sufficient; however, if UPC >2, an additional collection of 24-hour urine for UPC calculation was required.

Blood samples were collected for pharmacodynamic biomarker assessments.

### Response/Efficacy Assessments

Response assessment included SPEP/immunofixation, UPEP/immunofixation (in patients with a baseline urine M protein ≥200 mg/24 hour or for assessment of VGPR or better), SFLC, quantitative immunoglobulins, and plasmacytoma evaluation by imaging (at baseline, every 4 cycles, and at additional time points if clinically indicated). These samples were collected for local assessment. In addition, blood were analyzed in the central laboratory using a modified SPEP for patients with IgG myeloma.

Bone marrow aspirate, including a BM aspirate clot, and biopsy were required as part of the baseline visit, as well as on Day 4 of Cycle 1 (in expansion cohort only, contingent upon activity observed during dose escalation or emerging during dose expansion), Day 22-28 of Cycle 2, and to confirm CR in patients negative for blood and urine M protein. In monotherapy intensive dosing and combination therapy, bone marrow aspirate and biopsy were also required in Cycle 6 and every 6 cycles thereafter. Both bone marrow aspirate and biopsy samples were assessed locally at the site for clinical evaluation (with the exception of Cycle 1 Day 4 specimen). In addition, biomarker analyses were performed centrally on these samples. Any additional bone marrow aspirates and biopsies collected at any other time while on the trial may also be submitted for central assessment.

The bone marrow specimens were tested centrally for assessment of response/resistance to SEA-BCMA and could include but are not limited to: evaluation of BCMA expression, immune activation, disease risk profiling, gene expression profiling, and minimal residual disease (MRD) assessment.

The determination of antitumor activity were based on response assessments made according to the 2016 IMWG Criteria (Kumar et al., Lancet Oncol 17(8): e328-46, 2016) and treatment decisions by the investigator were based on these assessments. Clinical response of sCR, CR, VGPR, PR, SD, and PD were determined at each assessment based on local laboratory (and the modified SPEP run by the central laboratory for patients with IgG MM), radiological, and clinical evaluations. Progressive disease was based on IMWG 2016 criteria and/or clinical disease progression per investigator. All IMWG responses were confirmed responses. When applicable, determination of immunophenotypic CR, MRD status, and minimal response were made per the IMWG 2016 criteria.

### Pharmacokinetic and Immunogenicity Assessments

Blood and bone marrow samples for PK and ATA assessment were collected. Qualified assays were used to measure concentrations of SEA-BCMA in serum and bone marrow and ATA in serum. Remaining PK samples were archived for possible analysis of SEA-BCMA-related species. The assays included enzyme-linked immunosorbent assays (ELISA) assay, as well as other assays if further characterization was required.

A qualified electrochemiluminescence assay were used to assess ATA.

### Biomarker Studies

Peripheral blood and bone marrow samples for biomarker analyses were collected at time points outlined in the following sections. In addition to protocol-mandated collections of tumor specimens, bone marrow specimens collected at the discretion of the investigator could be submitted for central biomarkers analysis. For all bone marrow collections, sites supplied bone marrow aspirates as well as bone marrow biopsy specimens and bone marrow aspirate clot specimens as formalin-fixed, paraffin-embedded (FFPE) blocks. For samples acquired for SOC, unstained slides might be submitted if an FFPE block for the bone marrow biopsy or clot were not available. Samples were sent to the central lab for analysis as described in the laboratory manual.

Samples were evaluated for expression of BCMA and relevant biomarkers that might be associated with the activity of SEA-BCMA and/or change in response to treatment. Analysis of tumor tissue and peripheral blood could also include markers associated with prognosis, response, or resistance. Changes in peripheral blood immune cell subsets were measured as potential pharmacodynamic and safety markers.

### Genetic Profiling of Effector Cells

Small nucleotide polymorphisms of FcγRII and FcγRIII, which may influence the response to SEA-BCMA, were determined, including, but not limited to, testing of the following polymorphisms:

FCGRIIIA—158V/F

FCGRIIA—131H/R

### Serum Free Light Chain and Modified SPEP

Kappa and lambda free light chains were quantified in serum of patients as surrogate markers of antitumor activity.

For patients with IgG myeloma who have low levels of serum M-protein SPEP, a reflex modified SPEP assay were used to assess for residual serum M-protein in the absence of interference from SEA-BCMA.

### Peripheral Blood Immunophenotyping

Peripheral blood samples were collected for evaluation of circulating immune cells by flow cytometry. Changes in circulating immune cell subsets were measured as potential pharmacodynamic markers of SEA-BCMA activity. Flow cytometry measurements included, but not be limited to, characterizing NK cells, monocytes, T cells, and B cells.

### Plasma Cytokines/Chemokines

The levels of circulating cytokines/chemokines may be assessed by ELISA and/or multiplex cytokine/chemokines assays.

### Soluble Target and Ligands

The levels of circulating soluble BCMA (sBCMA), APRIL and BAFF may be assessed by ELISA or other methods (e.g., LC-MS or flow cytometry).

### Plasma Biomarkers and PBMCs

Plasma and PBMCs were collected for retrospective analyses of cellular and circulating biomarkers associated with response and/or resistance to SEA-BCMA.

### Characterization of Tumor Tissue

Baseline and on-treatment bone marrow aspirates and biopsies were collected to assess disease relevant immune subsets, characterize tumor burden, investigate depth of response and determine prognostic signatures and response to treatment. Additional protein, gene expression profiling, as well as further molecular characterization of the tumor for myeloma disease relevant risk markers, may also be evaluated to identify biomarkers predictive of response or resistance to SEA-BCMA.

### Bone Marrow Immunophenotyping

Expression of BCMA on tumor plasma cells, as well as presence and changes of immune components in the bone marrow, may be evaluated by flow cytometry and/or immunohistochemistry.

### Gene Expression Profiling/NGS/FISH

Baseline and treatment-related changes in gene expression profiles in tumor and tumor microenvironment may be assessed by RNA sequencing of tumor (CD138-positive) and non-tumor (CD138-negative) cells purified from bone marrow aspirates, to determine prognostic disease-risk signatures as well as baseline characteristics and on-treatment changes that may correlate with response or resistance. Cytogenetic analyses or DNA sequencing of CD138-positive plasma cells enriched from bone marrow aspirate collected at Baseline may also be carried out to further determine genetic changes that may predict or be associated with response to SEA-BCMA.

### MRD

MRD evaluation using the Adaptive NGS for MRD assay (Martinez-Lopez et al., Blood 123(20): 3073-9, 2014) may be carried out on relevant specimens to understand the activity of SEA-BCMA.

### Bone Marrow Plasma

Bone marrow plasma were collected and may be tested for levels of soluble target, ligands, and/or cytokines/chemokines that may influence or correlate with response to SEA-BCMA.

### Adverse Events

According to the International Council for Harmonisation (ICH) E2A guideline Definitions and Standards for Expedited Reporting, and 21 CFR 312.32, IND Safety Reporting, an AE is any untoward medical occurrence in a patient or clinical investigational subject administered a medicinal product and which does not necessarily have a causal relationship with this treatment.

In general, an abnormal laboratory value should not be recorded as an AE unless it is associated with clinical signs or symptoms, requires an intervention, results in a SAE, or results in study termination or interruption/discontinuation of study treatment (SEA-BCMA and/or dexamethasone). When recording an AE resulting from a laboratory abnormality, the resulting medical condition rather than the abnormality itself should be recorded (e.g., record “anemia” rather than “low hemoglobin”).

### Serious Adverse Events

An AE was classified as an SAE if it met one of the following criteria:
Fatal: AE resulted in death
Life threatening: The AEs placed the patient at immediate risk of death. This classification does not apply to an AE that hypothetically might cause death if it were more severe.
Hospitalization: The AE resulted in hospitalization or prolonged an existing inpatient hospitalization. Hospitalizations for elective medical or surgical procedures or treatments planned before the signing of informed consent in the study or routine check-ups are not SAEs by this criterion. Admission to a palliative unit or hospice care facility is not considered to be a hospitalization. Hospitalizations or prolonged hospitalizations for scheduled therapy of the underlying cancer or study target disease need not be captured as SAEs.
Disabling/incapacitating: An AE that resulted in a persistent or significant incapacity or substantial disruption of the patient's ability to conduct normal life functions.
Congenital anomaly or birth defect: An adverse outcome in a child or fetus of a patient exposed to the molecule or study treatment regimen before conception or during pregnancy.

Medically significant: The AE did not meet any of the above criteria, but could have jeopardized the patient and might have required medical or surgical intervention to prevent one of the outcomes listed above or involves suspected transmission via a medicinal product of an infectious agent. Potential drug-induced liver injury (DILI) also is considered a medically significant event.

### Adverse Event Severity

AE severity was graded using the NCI-CTCAE, version 4.03.

AE severity and seriousness were assessed independently. ‘Severity’ characterizes the intensity of an AE. ‘Serious’ is a regulatory definition and serves as a guide to the sponsor for defining regulatory reporting obligations.

### Relationship of the Adverse Event to Study Treatment

The relationship of each AE to each study treatment (SEA-BCMA and/or dexamethasone) were evaluated by the investigator using the following criteria:
Related: There is evidence to suggest a causal relationship between the drug and the AE, such as:
   A single occurrence of an event that is uncommon and known to be strongly associated with drug exposure (e.g., angioedema, hepatic injury, Stevens-Johnson Syndrome)
   One or more occurrences of an event that is not commonly associated with drug exposure, but is otherwise uncommon in the population exposed to the drug (e.g., tendon rupture)
Unrelated: Another cause of the AE is more plausible (e.g., due to underlying disease or occurs commonly in the study population), or a temporal sequence cannot be established with the onset of the AE and administration of the study treatment, or a causal relationship is considered biologically implausible

### Data Analysis Methods

### Determination of Sample Size

Approximately 65 patients were enrolled in SEA-BCMA monotherapy studies. This number was based on the assumption that approximately 25 patients were evaluated in dose-escalation and that approximately 40 patients were evaluated in an expansion cohort at the MTD or optimal dose to further define the safety and antitumor activity of SEA-BCMA.

Operating characteristics of the dose escalation part of the study, including the average number of patients allocated to each dose across a variety of toxicity scenarios are presented in the simulation report.

No formal hypothesis test was planned for the expansion cohorts. Assuming a 30% ORR, the 95% exact confidence interval (CI) is (17%, 47%) and the 80% exact CI is (20%, 41%) with 40 patients.

No formal hypothesis is planned for intensive dosing monotherapy and combination therapy. Assuming the observed ORR is between 30%-50%, the 95% binomial exact CIs are summarized in Table 9 below.

TABLE 9
95% binomial exact CIs

| ORR | 95% CI (N = 20) | 95% CI (N = 40) |
|---|---|---|
| 30% | 12%, 54% | 17%, 47% |
| 40% | 19%, 64% | 25%, 57% |
| 50% | 27%, 73% | 34%, 66% |

### Objective Response Rate

A patient was determined to have an OR if, based on the 2016 IMWG uniform response criteria, they achieve a sCR, CR, VGPR, or a PR. The ORR is defined as the proportion of patients with an OR per investigator. Patients whose disease response could not be evaluated per the 2016 IMWG uniform response criteria were scored as Not Evaluable for calculating the ORR. Patients who do not have post baseline response assessment, or the response is Not Evaluable per IMWG criteria were counted as non-responders in calculation of ORR.

### Complete Response Rate

A patient was determined to have a CR if, based on the 2016 IMWG uniform response criteria they achieve a sCR or CR. The CR rate is defined as the proportion of patients with a CR per investigator. Patients whose disease response cannot be evaluated per the IMWG uniform response criteria were scored as Not Evaluable for calculating the CR rate.

### Duration of Objective Response

Duration of OR was defined as the time from first documentation of OR (sCR, CR, VGPR, or PR) to the first documentation of disease progression or to death due to any cause, whichever comes first. Disease progression includes objective evidence of tumor progression (based on serum, urine, or bone marrow assessments) and/or clinical progression per investigator. Duration of response were censored on the date of the last disease assessment documenting absence of PD for patients who do not have disease progression and are still on study at the time of an analysis, or are removed from study prior to documentation of tumor progression. Patients who have started a new antitumor treatment prior to documentation of PD were censored at the last disease assessment prior to start of new treatment. Duration of response were only calculated for the subgroup of patients achieving a sCR, CR, VGPR, or PR.

### Duration of Complete Response

Duration of CR is defined as the time from first documentation of complete response (sCR, CR) to the first documentation of disease progression or to death due to any cause, whichever comes first. Disease progression includes objective evidence of tumor progression (based on serum, urine or bone marrow assessments) and/or clinical progression per investigator. Duration of CR was censored on the date of the last disease assessment documenting absence of PD for patients who do not have disease progression and were still on study at the time of an analysis, or were removed from study prior to documentation of tumor progression. Patients who have started a new antitumor treatment prior to documentation of PD were censored at the last disease assessment prior to start of new treatment.

Duration of CR were only calculated for the subgroup of patients achieving a sCR or CR.

### Progression-Free Survival

PFS is defined as the time from the start of any study treatment to first documentation of disease progression or to death due to any cause, whichever comes first. Disease progression includes objective evidence of tumor progression (based on serum, urine or bone marrow assessments) and/or clinical progression per investigator. PFS were censored on the date of the last disease assessment documenting absence of progressive disease (PD) for patients who do not have disease progression and were still on study at the time of an analysis, or are removed from study prior to documentation of tumor progression. Patients who have started a new antitumor treatment prior to documentation of PD were censored at the last disease assessment prior to start of new treatment. Patients lacking an evaluation of tumor response after their first dose had their event time censored at 1 day.

### Overall Survival

OS is defined as the time from the start of any study treatment to the date of death due to any cause. Specifically: OS=date of death−date of first dose of any study treatment+1.

OS for patients who were alive at their date of last contact, including those lost to follow-up, were censored at the date of last contact. If the last recorded date where a patient was known to be alive was the date of first dose of any study treatment, survival time would be censored on the date of first dose of any study treatment (i.e., OS duration of 1 day).

### MRD-Negativity Rate

The rate of MRD negativity will be reported among patients who achieved VGPR or better.

### Efficacy Analyses

All efficacy analyses were presented using the All Treated Patients set. Selected efficacy endpoints were also presented using the EE analysis set. The observed ORR and CR rate and corresponding 95% CIs were presented. Patients whose disease response cannot be assessed will be counted as non-responders. Patients with intrapatient dose escalation prior to achieving a response were counted as non-responders at their initial dose.

Duration of response, PFS and OS were estimated using Kaplan-Meier methodology, and Kaplan-Meier plots will be provided. Medians were calculated, where possible. The 95% CIs were also calculated, as appropriate.

### Pharmacokinetic and Immunogenicity Analyses

The PK of SEA-BCMA were evaluated by noncompartmental analysis. The following PK parameters were determined where data allow:

Area under the curve

Concentration at the end of infusion (Cₑₒᵢ) or maximum observed concentration (Cₘₐₓ)

Trough concentration (C_{trough})

Terminal or apparent terminal half-life (t_{1/2})

Systemic clearance and volume of distribution at steady state

Accumulation ratio

### Biomarker Analyses

Peripheral blood and bone marrow aspirates and biopsies were collected for biomarker assessments. Assessments performed with these samples included, but are not limited to, myeloma cell monitoring and profiling, including expression of BCMA and assessments of immune cell populations. Additionally, bone marrow samples are analyzed to identify gene expression profiles, cytogenetic abnormalities, genetic mutations, and other tumor and tumor microenvironment-related biomarkers that may define disease risk profiles, predict response to SEA-BCMA, and clarify SEA-BCMA mechanisms of action. MRD are analyzed in selected bone marrow specimens using next generation sequencing (NGS). Plasma and serum were also collected for quantification of biomarkers of drug activity, which included sFLC, cytokines/chemokines, soluble BCMA, and other soluble biomarkers.

Relationships of biomarker and pharmacodynamic parameters (e.g., baseline values, absolute and relative changes from baseline) to efficacy, safety and PK parameters were explored. Relationships and associated data that were determined to be of interest were summarized.

### Example 2. Pharmacokinetic Analysis of SEA-BCMA

To investigate the pharmacokinetic parameters of SEA-BCMA, various doses of SEA-BCMA were administered and serum concentrations were evaluated at different time points. Specifically, a dose of 100 mg, 200 mg, 400 mg, 800 mg, or 1600 mg of SEA-BCMA was injected at Day 0, and repeated at Day 14 post-injection. A stepwise IV infusion was used for the administration, starting with 50 mg/hr (30 mins), which was incrementally increased (2-fold) to a maximal rate of 400 mg/hour.

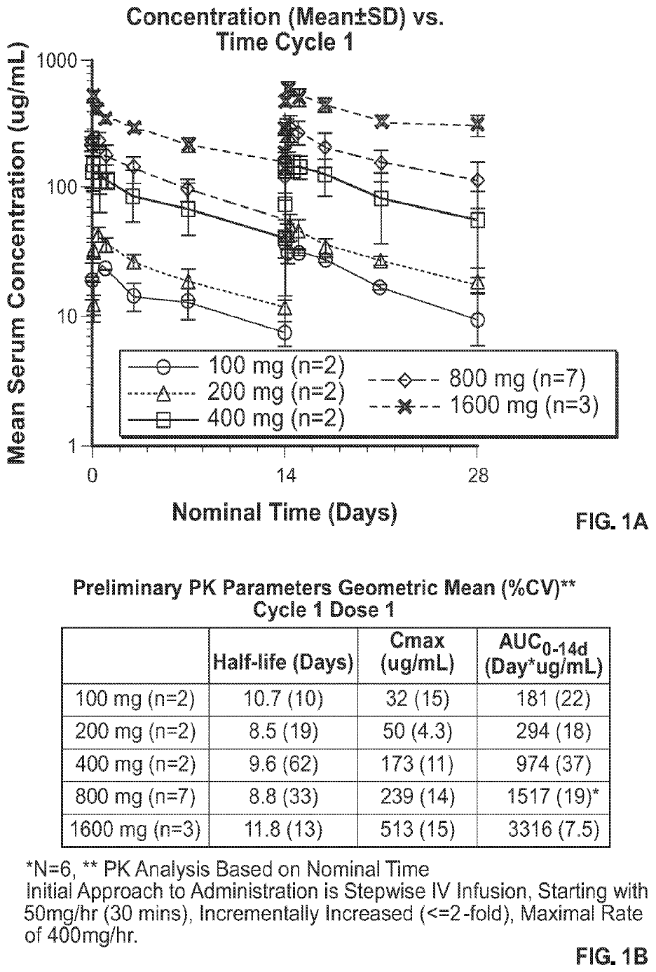

As shown in FIG. 1A, the post-administration serum concentration of SEA-BCMA is dose proportional over the evaluated range of 100 mg-1600 mg. FIG. 1B shows the half-life, maximum concentration (Cₘₐₓ), and area under the curve (AUC) of the antibody for each administered dose. The pharmacokinetics analysis represent total circulating SEA-BCMA in the serum sample. As can be seen from these figures, the half-life for SEA-BCMA from these studies was approximately 10 days, and PK reaches steady state at cycle 3 with approximately 70% accumulation after Q2W dosing. No anti-SEA-BCMA antibodies were found in any of the samples tested.

### Example 3. Estimation of Free SEA-BCMA Using In Vitro Cell Binding Capacity Assay

An in vitro cell binding capacity assay was developed to complement serum concentration measurements of total SEA-BCMA, total soluble BCMA, and flow cytometric evaluation of free, unbound BCMA and total BCMA on plasma cells from the bone marrow. The assay measures the ability of free, unbound SEA-BCMA (e.g. not bound by sBCMA) from patient serum to bind and saturate BCMA in-vitro on a BCMA positive cell line. Titrations of known SEA-BCMA concentrations allow for an evaluation of the concentrations of free, unbound SEA-BCMA in patient serum. Together with a measurement of the percent-bound BCMA on the cell surface, this provides a direct indication of the ability of dosed SEA-BCMA to bind and saturate cell membrane-bound BCMA.

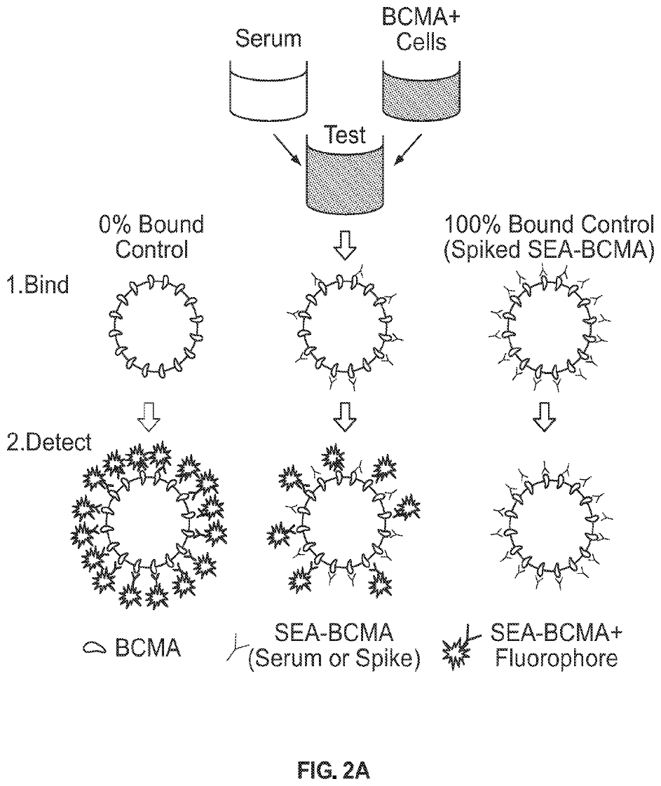

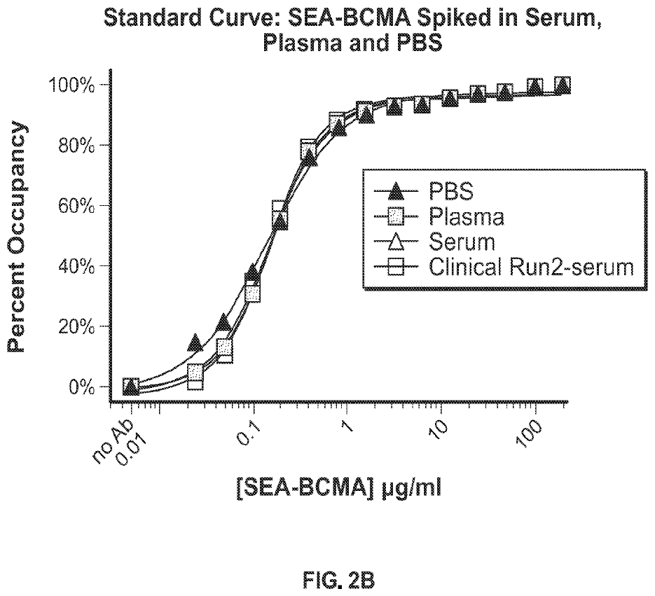

As shown in FIG. 2A, a suspension of cultured MM1R cells was pelleted and re-suspended in serum from peripheral blood of subjects collected at Cycle 1, Day 1 (C1D1) pre-dose, C1D1 EOI (end of infusion), C1D2, C1D8, C1D15 pre-dose, C1D15 EOI, C1D16, C1D22, C2D1 EOI, C2D15 pre-dose, and C3D1 pre-dose. After incubation at room temperature for ½ hour, the cells were washed and stained with a saturating amount of SEA-BCMA conjugated to a fluorescent dye. After incubation at 4° C. in the dark for ½ hr, the cells were washed and fixed. Stained cells were analyzed on an Invitrogen Attune NxT flow cytometer. FlowJo V10 software was used to gate on viable cells and record the median fluorescent intensity (MFI). GraphPad Prism 8 was used for analysis.

As shown in FIG. 2B, levels of free SEA-BCMA that are able to bind membrane BCMA are estimated using standard curves showing occupancy of cell-surface BCMA under different concentrations of SEA-BCMA.

The recorded MFI was normalized as follows. Normalization to 0% binding is the MFI from a control well incubated in healthy human serum with no SEA-BCMA present. Normalization to 100% binding is the MFI from a control well incubated in healthy human serum spiked with SEA-BCMA to a concentration 200 μg/mL. A titration of SEA-BCMA in healthy human serum may be used to generate a standard curve using the known amounts of SEA-BCMA.

### Example 4. Maintenance of Free SEA-BCMA in Patients

The maintenance of free SEA-BCMA in the patient serum that are able to bind in vitro to the cell-surface BCMA of a BCMA-expressing cell line was investigated. Specifically, two doses of 100 mg, 200 mg, 400 mg, 800 mg, or 1600 mg of SEA-BCMA were administered to each patient, and percent occupancies of cell-surface BCMA were evaluated.

In a first experiment, a first dose of 100 mg, 200 mg, or 400 mg of SEA-BCMA was administered to each patient, followed by a same dose of SEA-BCMA to each patient, 14 days after the first dose.

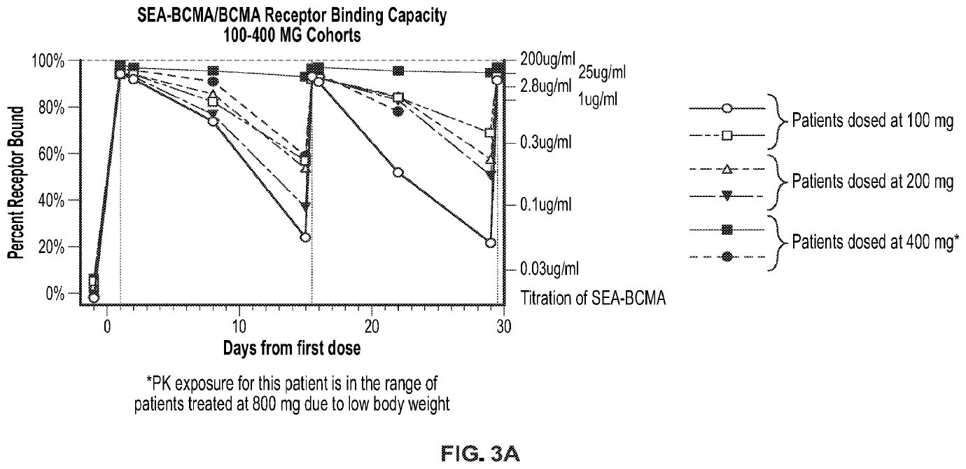

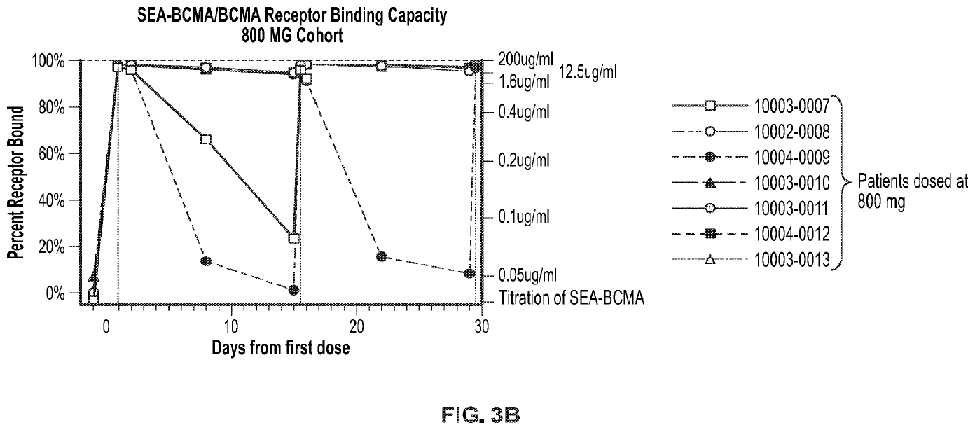

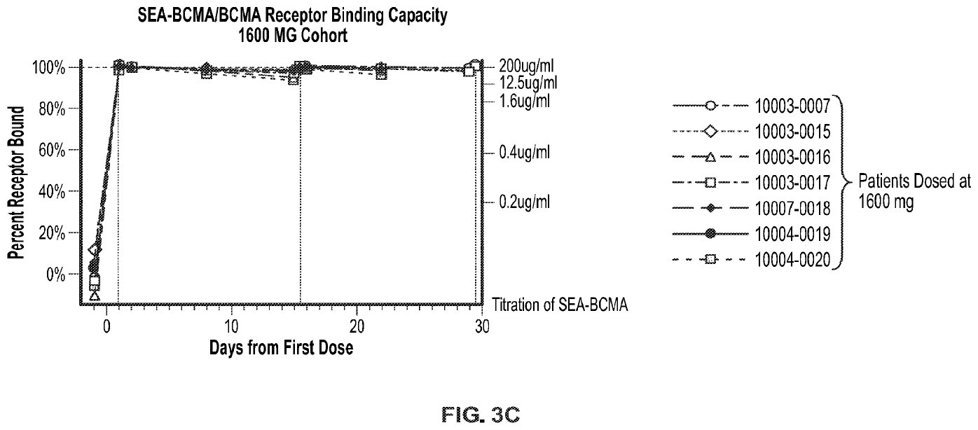

As shown in FIG. 3A, 100 mg-400 mg serum levels of SEA-BCMA were not able to maintain >95% bound BCMA in vitro through the dosing intervals shown.

In a second experiment, a first dose of 400 mg or 800 mg of SEA-BCMA was administered to each patient, followed by a second dose of 400 mg or 800 mg of SEA-BCMA, 14 days after the first dose. As shown in FIG. 3B, high levels of free SEA-BCMA were maintained in patients serum throughout the dosing period in the majority of patients (6/8) dosed at 800 mg (or equivalent). The pharmacokinetic exposure of SEA-BCMA for one patient was in the range of patients treated with 800 mg doses due to low body weight. One patient was treated with a first dose of 800 mg of SEA-BCMA and a second dose of 400 mg of SEA-BCMA due to infusion-related reactions (IRR).

In a third experiment, a first dose of 1600 mg of SEA-BCMA was administered to each patient, followed by a second dose of 1600 mg of SEA-BCMA, 14 days after the first dose. As shown in FIG. 3C, high levels of free SEA-BCMA in serum were maintained throughout the dosing period in all of the patients (7/7) dosed at 1600 mg.

These data demonstrate that a dosing regimen of 1600 mg of SEA-BCMA administered every two weeks generates levels of serum SEA-BCMA able to support in vitro saturation of membrane BCMA of a BCMA-expressing cell line, which suggests that target BCMA saturation throughout the dosing interval may be occurring in vivo at 1600 mg dose.

### Example 5. Flow Cytometric Assessment of BCMA Expression and Engagement on MM Cells in the Bone Marrow

A Flow Cytometric Assay was designed to identify multiple myeloma cells in patients bone marrow aspirate, and measure levels of BCMA (unbound and total) and APRIL on the MM cell surface. Detection of unbound membrane BCMA demonstrates presence of BCMA that is not bound by APRIL, BAFF or SEA-BCMA, and therefore incomplete target engagement on treatment.

Bone marrow aspirates were collected from enrolled patients at Baseline and on-treatment (C2D22, confirmation of CR, per standard of care), and tested by flow cytometry with one day of collection in most cases.

MM cells detection was performed using extracellular CD138, CD38, CD45, CD56 and CD28 staining and intracellular kappa and lambda light chains staining. Profiling of BCMA expression was performed using 2 anti:BCMA antibodies: BCMA available to binding for SEA-BCMA is detected using labeled 16.17 IgG1 (parent antibody to SEA_BCMA), while total extracellular BCMA is detected using labeled 16.16 anti-BCMA Ab (Seattle Genetics) that does not compete with SEA-BCMA or APRIL binding to BCMA. Detection of APRIL, most probably bound to BCMA on the MM cell surface, is also performed.

Each sample was split into 3 aliquots: one aliquot stained using only the MM gating antigens but no anti-BCMA or anti-APRIL antibodies (gating control), one aliquot stained with MM Gating antigens and the two labeled anti-BCMA antibodies, and one incubated for 2 hours at 37 C with spiked BCMA (100 ug/mL) before staining with MM gating antigens+APRIL+labeled non-competing anti-BCMA 16.16 antibody. After staining, washing and fixation in 2% paraformaldyde, cells were analyzed on a BD FACScanto cytometer, with up to 2×10⁶ leukocytes acquired. Data analysis was performed with FACS Diva (Q2) and FlowJo (Seattle Genetics).

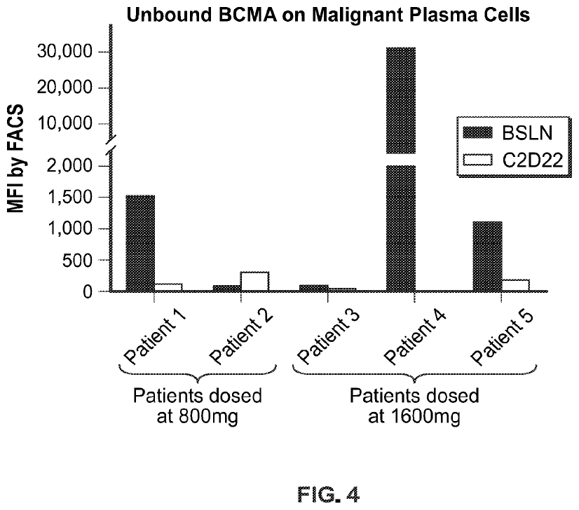

As shown in FIG. 4, SEA-BCMA engaged BCMA on MM cells in bone marrow of patients treated in dose escalation and reduced levels of unbound membrane BCMA on malignant plasma cells. Incomplete saturation of membrane BCMA was observed in evaluable patients dosed at 800 mg and 1600 mg, while complete saturation was observed in at least one patient dosed at 1600 mg of SEA-BCMA.

### Example 6. Patient Case Study

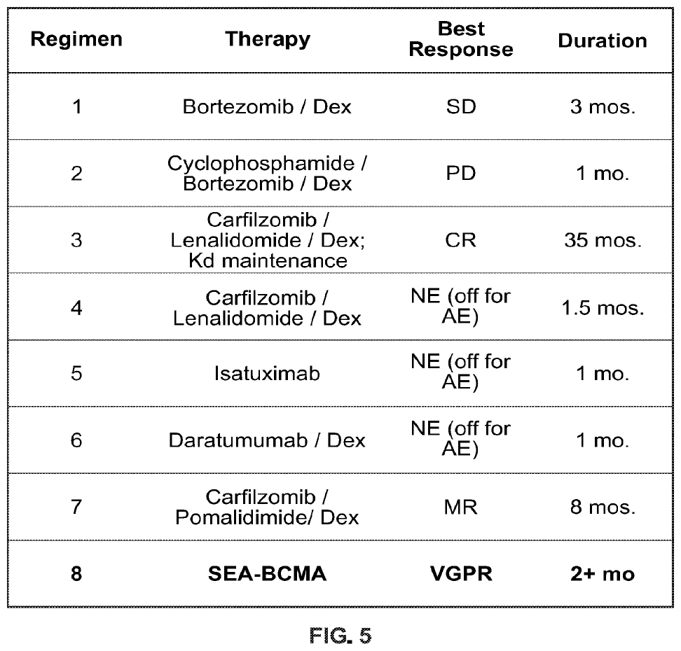

One patient enrolled in the study is a 83 year old male. The patient has an ECOG (Eastern Cooperative Oncology Group, Zubrod, World Health Organization) performance status 1, and has an IgG lambda myeloma with gain (1q21). Prior to treatment with SEA-BCMA, the patient had received 7 lines of treatment, which are specified in FIG. 5. At the beginning of the current study, the patient had the following status: SPEP 0.33, UPEP unmeasurable, BMA 9% plasma cells, no plasmacytomas, lambda FLC 52.68 mg/dL. In the current study, the patient received a regimen of 1600 mg of SEA-BCMA every two weeks for at least two months.

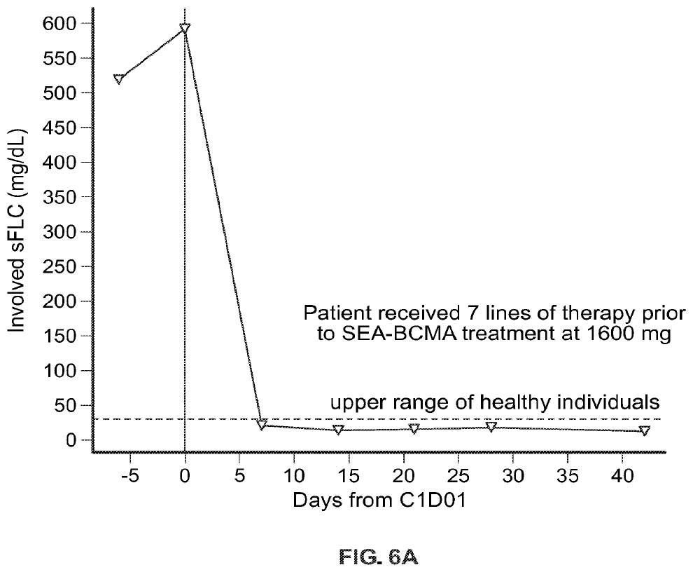

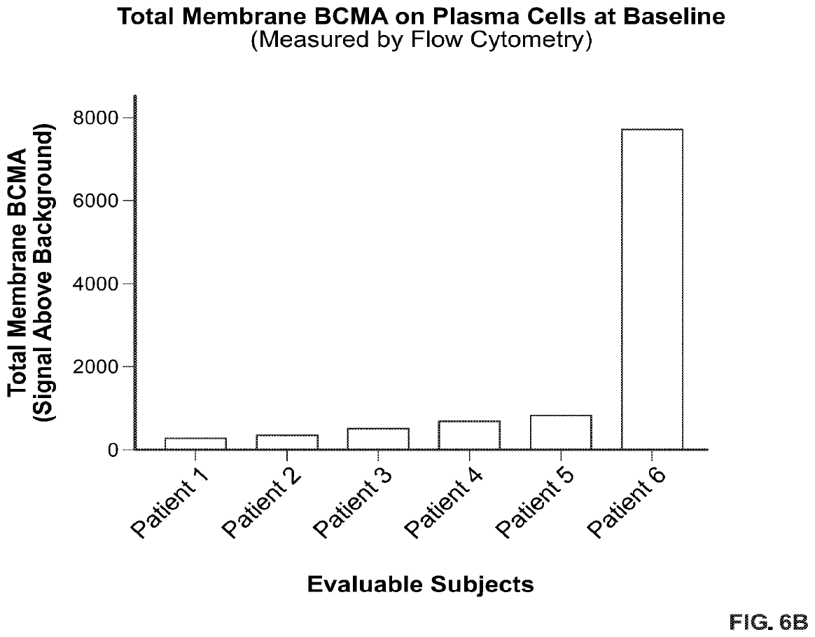

The level of serum free light chain (sFLC) was evaluated as an index of plasma cell disorders. As shown in FIG. 6A, after one week of the first 1600 mg dose of SEA-BCMA, the sFLC level in the patient decreased significantly by about 90%, which is below the upper level of the range for a healthy individual. The low level of sFLC was maintained to at least Day 40 after the first dose. As shown in FIG. 6B, this patient demonstrates higher level of membrane-bound BCMA at baseline, compared to several other patients enrolled in the study. Safety and tolerability studies show that the patient developed a Grade 2 maculopapular rash during the second cycle of treatment with SEA-BCMA. The patient also had an extensive rash history with prior lines of therapy. Response assessment confirms that patient has very good partial response (VGPR) at the end of the second two-week cycle of SEA-BCMA treatment. These data indicate that SEA-BCMA was able to provide sustained very good partial response (VGPR) of multiple myeloma in a subject that was refractory to treatment with seven different types of previous treatment.

### Example 7. Further Patient Case Study

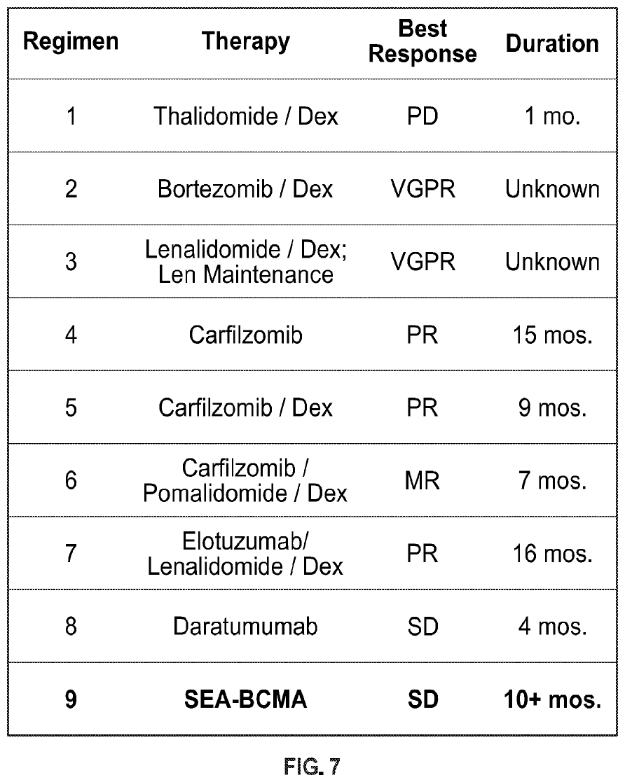

Another patient enrolled in the study is a 75 year old male. The patient has an ECOG (Eastern Cooperative Oncology Group, Zubrod, World Health Organization) performance status score of 1, and has an IgG kappa myeloma. Prior to treatment with SEA-BCMA, the patient had received 8 lines of treatment, which are specified in FIG. 7. At the beginning of the current study, the patient had the following status: SPEP 3.29, UPEP unmeasurable, BMA 40% plasma cells, no plasmacytomas. In the current study, the patient received a dose of 200 mg of SEA-BCMA every two weeks for cycles 1-3, a dose of 400 mg of SEA-BCMA every two weeks for cycles 4-8, a dose of 800 mg of SEA-BCMA every two weeks for cycle 9, and a dose of 1600 mg of SEA-BCMA for cycle 10.

Safety and tolerability studies show that the patient tolerated the therapy well without any adverse effects related to SEA-BCMA. These data demonstrate that SEA-BCMA was able to provide successful treatment of multiple myeloma in a subject who was refractory to treatment with eight previous lines of treatment, including maintaining stable disease even 10 months after commencement of treatment.

### Example 8. Clinical Trial Summary

In the clinical trial, 20 patients were treated across 5 dose levels, from a 100 mg flat-dose to a 1600 mg flat-dose. The treatment duration ranged from 1 cycle to 11 cycles, with a median duration on treatment of 2.5 cycles. The patients enrolled in the study were heavily pre-treated late-line myeloma patients. The median age of the patients were 70 years old. 89% of the patients had ECOG (Eastern Cooperative Oncology Group, Zubrod, World Health Organization) performance status scores of 1. The median number of prior lines of therapy the patients had was 5.

The dose escalation study has reached the highest pre-specified dose level, 1600 mg. The pharmacokinetics of SEA-BCMA is approximately dose-proportional (100-1600 mg), and the half-life of the antibody is about 10 days. These data provide preliminary evidence of monotherapy activity. For all dose levels, stable disease was observed after a minimum of 3 cycles of treatment. The high levels of circulating SEA-BCMA able to bind membrane BCMA was maintained throughout the Q2wk dosing interval in all patients treated with 1600 mg doses of SEA-BCMA, and in 6 out of 8 patients treated with 800 mg doses of SEA-BCMA (or equivalent).
Among 3 response evaluable patients treated with a dose of 1600 mg SEA-BCMA, 1 patient was confirmed with very good partial response (VGPR). The clinical trial data support a two-week (Q2wk) interval as likely appropriate for 1600 mg dosing of SEA-BCMA.

The tolerable safety profile showed that SEA-BCMA was well-tolerated with no severe adverse effects, except 1 case of dose-limiting toxicity (DLT) (Grade 3 infusion-related reactions) and 2 total serious infusion-related reactions, among 20 total patients treated. The infusion-related reactions were resolved.

### Example 9. Additional Preliminary Results Following Clinical Trial Progression

As the clinical trial described herein has progressed, additional findings have been made. In the dose escalation study part of the study, SEA-BCMA was administered once every 2 weeks with 100 mg per dose, 200 mg per dose, 400 mg per dose, 800 mg per dose, and 1600 mg per dose to different groups of patients. Patients that received the 1600 mg per dose exhibited the best response. The results indicate that 1600 mg is the selected dose for treatment because this dose provided the highest overall confirmed response rate and was still found to be safe. The result is surprising given the relatively large dose level of SEA-BCMA that can be safely administered. This highlights the tolerability of SEA-BCMA. As SEA-BCMA is well tolerated, it suggests that SEA-BCMA is suitable to be combined with other therapeutic agents.

In addition, results have been received for patients that were enrolled in the intensive dosing study. 1600 mg of SEA-BCMA was dosed once a week (q1wk) during the induction phase (for 8 doses during the first two 28-day cycles of therapy); following the induction phase, SEA-BCMA was dosed once every two weeks during the maintenance phase. The results indicate that intensive dosing was safe. Confirmed responses were also observed. These results confirm the high tolerability of SEA-BCMA and its potential to be safely combined with other therapeutic agents even at this dosing level.

Preliminary results were also received for patients that were enrolled in the dexamethasone combination therapy study. SEA-BCMA were administered on Day 1 and Day 15 of each 28-day cycle (standard dosing). Dexamethasone was administered on Day 1, Day 8, Day 15, and Day 22 of each 28-day cycle. The results indicate that the combination with dexamethasone was likewise found to be safe. Confirmed responses were also observed, suggesting the potential use of this particular combination in treating MM.

### Example 10. SEA-BCMA Displays Enhanced Activity in the Presence of Gamma Secretase Inhibition

Gamma secretase inhibitors (GSIs) have been shown to block BCMA cleavage and thus increase BCMA expression on the surface of cells (Laurent S A, Hoffmann F S, Kuhn P H, et al. γ-Secretase directly sheds the survival receptor BCMA from plasma cells. Nat Commun. 2015; 6:7333). The current inventors hypothesized that gamma secretase inhibition can improve SEA-BCMA activity on multiple myeloma (MM) cells. As shown in the experiments below, treatment of MM cells in vitro with nirogacestat (purchased from SelleckChem) or DAPT (EMD Millipore), or some other GSIs, enhances SEA-BCMA FcγRIII engagement and antibody dependent cellular cytotoxicity (ADCC). These data suggest the combination of SEA-BCMA with GSI inhibitors in the clinic can lead to greater anti-myeloma activity. Nirogacestat also increases BCMA NF-kB signaling, likely due to increased BCMA expression. SEA-BCMA can block this increased BCMA signaling in MM cells. These data collectively suggest that blocking of proliferative cell signaling by SEA-BCMA can contribute to anti-myeloma activity even in the presence of GSIs in the clinic.

SEA-BCMA Displays Enhanced FcγRIII Activation in the Presence of Gamma Secretase Inhibition

Experiments were first performed to test the impact of GSIs on the primary mechanism of action of SEA-BCMA, namely ADCC activity. The induction of FcγRIII signaling that initiates ADCC when SEA-BCMA is combined with immune effectors was examined first.

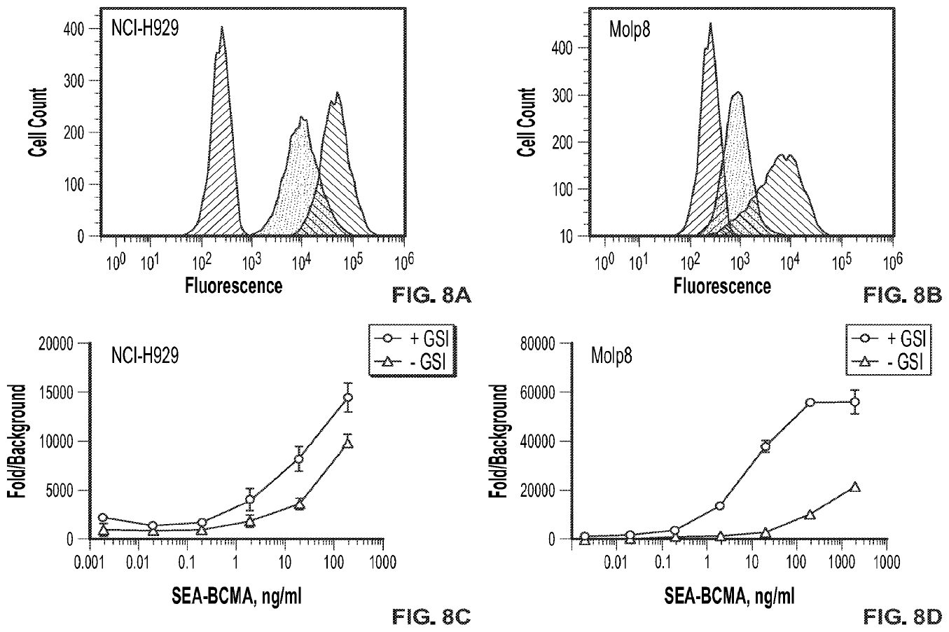

NCI-H929 or Molp-8 MM target cells were incubated with and without 1 μM DAPT for 24 hrs. Cells displayed increased BCMA expression using flow cytometry after incubation with DAPT (FIGS. 8A-8B). FcγRIII signaling was determined using a surrogate assay as manufacturer describes (Promega ADCC reporter bioassay cat #G9302). Cells were bound with antibody dose titrations+/−GSI for 30 minutes at 37° C. CD16A-Jurkat effector cells were then added with a 6:1 effector-to-target cell ratio. After an overnight incubation, the assay was developed with Bio-Glo and relative luminescence units (RLU) were measured on an Envision plate reader. Increased FcγRIII signaling was observed in the presence of the GSI (FIGS. 8C-8D). Thus, as shown in FIGS. 8A-8D, DAPT treatment can induce increased BCMA expression and increased FcγRIII signaling on NCI-H929 and Molp-8 cells. SEA-BCMA is a nonfucosylated antibody that displays enhanced FcγRIII binding affinity and induced signaling in comparison to fucosylated anti-BCMA antibodies. Enhanced signaling is the first step in the primary mechanism of action of SEA-BCMA. This signaling can translate to increased anti-MM cell lysis through ADCC. These data indicate that a GSI can potentially improve SEA-BCMA clinical activity.

### SEA-BCMA Displays Enhanced ADCC in the Presence of Gamma Secretase Inhibition

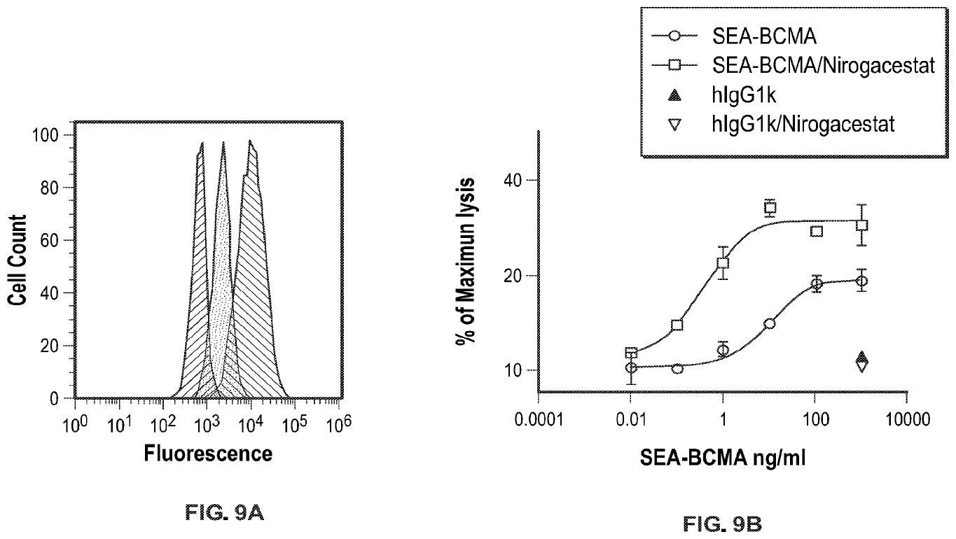

Whether the enhanced FcγRIII signaling translated to increased lysis of MM target cells by SEA-BCMA was determined. Molp-8 MM target cells were incubated with and without 0.2 μM Nirogacestat (purchased from SelleckChem) for 24 hrs. Cells were then Na₂ [⁵¹Cr] O₄ labeled and added to titrations of SEA-BCMA, or isotype antibody control. Effector cells, NK cells enriched from normal donor PBMC, were added at an effector-to-target cell ratio of 10:1 (50,000:5000). Donor NK cells were of the high affinity FcγRIII V/V genotype. The combination of antibodies, NK cells and target cells were incubated for 4 h at 37° C. with and without Nirogacestat. The radioactivity released into the culture supernatant of lysed target cells was then measured and the percent specific cell lysis calculated. U266 displayed increased BCMA expression using flow cytometry after incubation with Nirogacestat (FIG. 9A). Increased ADCC was observed in the presence of the GSI (FIG. 9B). This is the primary of mechanism of action of SEA-BCMA and translates to specific enhanced anti-MM lysis. It is expected that this will translate to improvements in anti-MM activity in the clinic when SEA-BCMA is combined with GSIs.

### Example 11. SEA-BCMA Partially Blocks NF-κB Signaling Induced by Gamma Secretase Inhibition

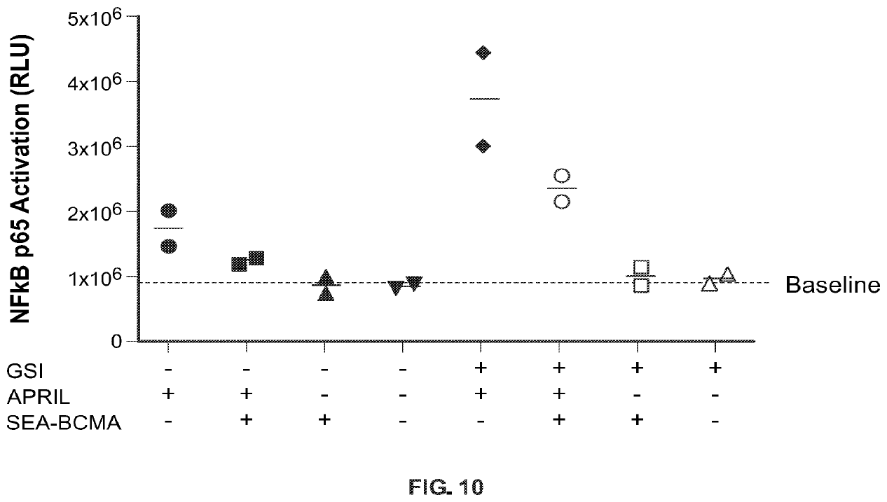

The secondary mechanism of action of SEA-BCMA is to block BCMA proliferative cell signaling. Increased BCMA from GSI treatment is expected to induce increased BCMA signaling. Therefore, the block of this enhanced signaling by SEA-BCMA was tested. BCMA-expressing NCI-H929 cells were serum-starved with and without 0.2 μM Nirogacestat (purchased from SelleckChem) for 16 hrs. Cells were then bound with and without 20 μg/mL SEA-BCMA and incubated with and without 1 μg/ml recombinant human APRIL (R&D Systems) for 20 minutes at 37° C. in the presence or absence of 0.2 μM Nirogacestat. 1 μg of nuclear extract was assayed in duplicate for NF-κB p65 activity by ELISA (TransAM NFκB Chemi p65, Active Motif). Relative Luminescence Units (RLU) were plotted showing increased NF-κB signaling from the GSI, Nirogacestat, which can be partially blocked by SEA-BCMA (FIG. 10). These data suggest that SEA-BCMA can continue to block MM proliferative signaling in the presence of GSIs in the clinic.

### Example 12. SEA-BCMA Combines Effectively with Daratumumab

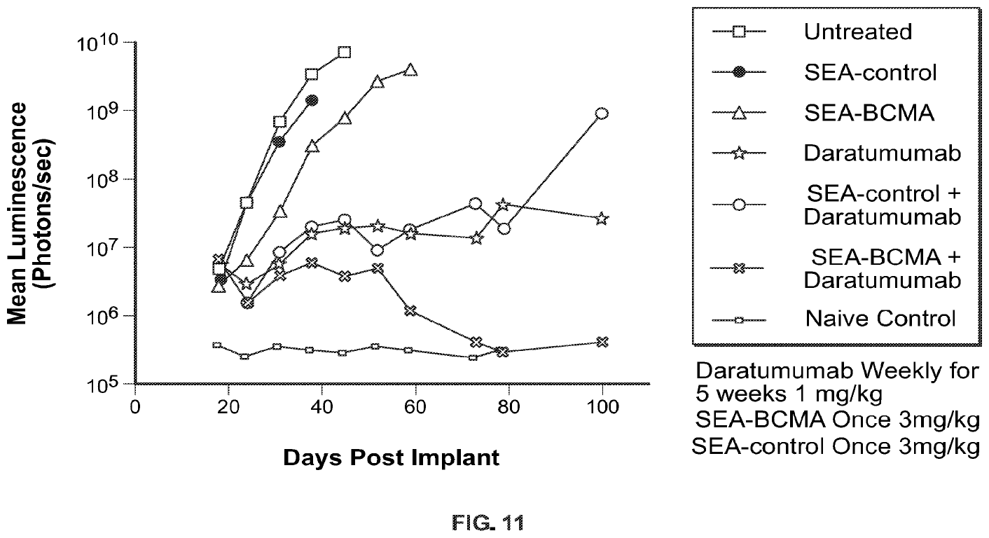

Experiments were performed to test whether SEA-BCMA can combine effectively with daratumumab in a disseminated MM mouse tumor xenograft. MOLP-8 luciferase cells were injected IV into SCID animals and allowed to home to the bone marrow. Molp-8 cells express 2000 copies of BCMA and 323,000 copies of CD38 (daratumumab target). When sufficient luminescence was observed, animals were dosed once IP with SEA-BCMA, or SEA non targeted control at 3 mg/kg. Daratumumab was dosed weekly 5× at 1 mg/kg. Median luminescence plotted, N=5. The combination of SEA-BCMA and daratumumab yielded complete remissions in 4 of 5 animals. Non targeted control combined with daratumumab was equivalent to daratumumab treatment alone. As shown in FIG. 11, SEA-BCMA combined with Daratumumab induced complete remissions in mice with MM tumor xenograft. These data support the use of a well-tolerated, antibody-based SEA-BCMA plus daratumumab combination regimen in the clinic.

### Example 13. SEA-BCMA Combines Effectively with the IMiD, Pomalidomide

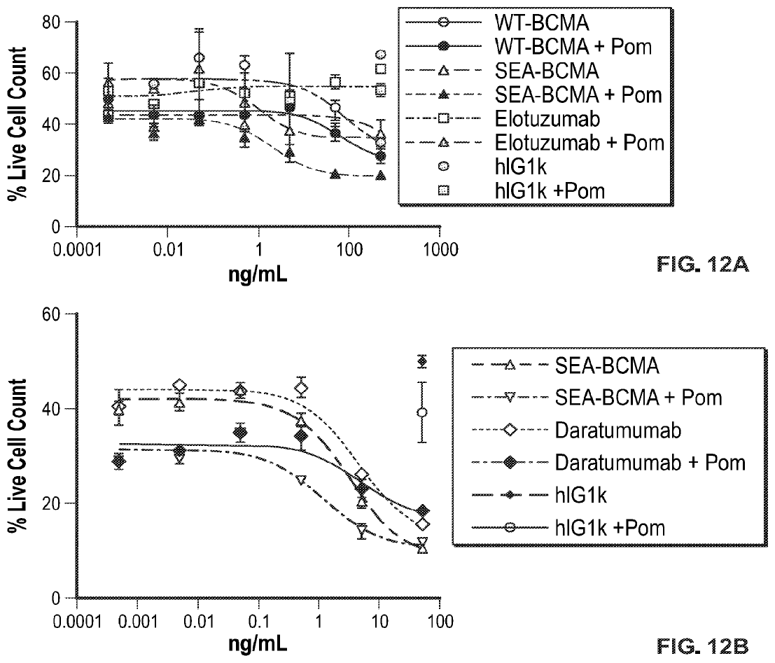

Experiments were performed to test whether SEA-BCMA combines effectively with pomalidomide in vitro by combining PBMC effector cells with MM1R target cells treated with both pomalidomide and SEA-BCMA or antibody controls. The first experiment (FIG. 12A) benchmarks against MM standard of care elotuzumab and nonfucosylated parent BCMA antibody. The second experiment (FIG. 12B) benchmarks against MM standard of care daratumumab. MM1R target cells express 15,000 copies of BCMA, 12,000 copies of CS1 (elotuzumab target), and 36,000 copies of CD38 (daratumumab target).

In the first experiment (FIG. 12A), normal donor PBMCs of the FcγRIII V/F genotype were mixed with CFSE (Invitrogen cat #V12883) labeled MM1R myeloma target cells at an effector-to-target cell ratio of 10:1. The mixture was treated with a dose range of the following antibodies: SEA-BCMA, elotuzumab, WT-BCMA (fucosylated parent Ab), or non-binding hIgG1k antibody control (Sigma cat #15154) with or without 3 μM pomalidomide (APEx Bio, cat #A4211). After 48 hrs, the % live MM1R cells were then determined using the viability dye 7-AAD (BD Biosciences, cat #559925) by flow cytometry. Fucosylated BCMA parent antibody was less active than the nonfucosylated version. Elotuzumab had no impact on cells unless pomalidomide was added. hIgG1k control antibodies also had no effect.

In the second experiment (FIG. 12B), normal donor PBMCs of the FcγRIII V/F genotype were treated with or without 0.5 μM pomalidomide (APEx Bio, cat #A4211) for 5 days. The resulting PBMCs were then washed and mixed with CFSE (Invitrogen cat #V12883) labeled MM1R myeloma target cells at an effector-to-target cell ratio of 10:1. The mixture was then treated with a dose range of the following antibodies: SEA-BCMA, daratumumab, or non-binding hIgG1k antibody control (Sigma cat #15154) with or without 0.5 μM pomalidomide (APEx Bio, cat #A4211) and incubated overnight. Percentage live MM1R cells were then determined using the viability dye 7-AAD (BD Biosciences, cat #559925) by flow cytometry. Daratumumab showed less activity than SEA-BCMA when combined with pomalidomide. hIgG1 control antibodies had no effect.

The results showed that the combination of SEA-BCMA with IMiD has improved target cell killing effects. These data suggest that SEA-BCMA plus IMiD combinations can lead to increased anti-myeloma activity in the clinic. Elotuzumab and daratumumab have been shown to combine productively with IMiDs in the clinic. These data at least suggest that SEA-BCMA can combine productively with IMiDs on par or better than elotuzumab or daratumumab.

What is claimed is:

1. A method of treating a subject having multiple myeloma (MM), the method comprising administering to the subject one or more doses of an antibody, or antigen binding fragment thereof, that specifically binds to a B cell maturation antigen (BCMA), and wherein the one or more doses are independently administered to the subject at about 100 mg of the antibody or antigen-binding fragment to about 2,000 mg of the antibody or antigen-binding fragment.

2. The method of claim 1, wherein the antibody or antigen-binding fragment thereof is a non-fucosylated antibody or antigen-binding fragment.

3. The method of claim 1 or 2, wherein a composition comprising the antibody or antigen-binding fragment thereof is administered to the subject, about or at least 95%, 97%, 98% or 99% of the antibody or antigen-binding fragment thereof in the composition are afucosylated.

4. The method of any one of claims 1-3, wherein the antibody or antigen binding-fragment thereof, comprises:
a heavy chain variable region comprising a CDR1 comprising SEQ ID NO: 1, a CDR2 comprising SEQ ID NO: 2, and a CDR3 comprising SEQ ID NO: 3, and
a light chain variable domain comprising a CDR1 comprising SEQ ID NO: 5, a CDR2 comprising SEQ ID NO: 6, and a CDR3 comprising SEQ ID NO: 7.

5. The method of any one of claims 1-4, wherein the antibody or the antigen-binding fragment thereof comprises a heavy chain variable domain comprising an amino acid sequence that is at least 80% identical to SEQ ID NO: 4 and a light chain variable domain comprising an amino acid sequence that is at least 80% identical to SEQ ID NO: 8.

6. The method of claim 5, wherein the antibody or the antigen-binding fragment thereof comprises a heavy chain variable domain comprising an amino acid sequence that is at least 90% identical to SEQ ID NO: 4 and a light chain variable domain comprising an amino acid sequence that is at least 90% identical to SEQ ID NO: 8.

7. The method of claim 6, wherein the antibody or the antigen-binding fragment thereof comprises a heavy chain variable domain comprising an amino acid sequence of SEQ ID NO: 4 and a light chain variable domain comprising an amino acid sequence of SEQ ID NO: 8.

8. The method of any one of claims 1-6, wherein the antibody or the antigen-binding fragment is humanized.

9. The method of any one of claims 1-8, wherein the antibody is an IgG1 antibody.

10. The method of any one of claims 1-8, wherein the antibody or antigen-binding fragment is not a bispecific antibody, a bispecific T cell engager (BiTE), a chimeric antigen receptor (CAR), or an antibody drug conjugate (ADC), or a portion thereof.

11. The method of any one of claims 1-10, wherein the one or more doses are independently administered to the subject at about 800 mg of the antibody or antigen-binding fragment to about 2,000 mg of the antibody or antigen-binding fragment.

12. The method of any one of claims 1-10, wherein the one or more doses are independently administered to the subject at about 1,200 mg of the antibody or antigen-binding fragment to about 2,000 mg of the antibody or antigen-binding fragment.

13. The method of any one of claims 1-10, wherein the one or more doses are independently administered to the subject at about 1,400 mg of the antibody or antigen-binding fragment to about 1,800 mg of the antibody or antigen-binding fragment.

14. The method of any one of claims 1-10, wherein the one or more doses are independently administered to the subject at about 400 mg of the antibody or antigen-binding fragment.

15. The method of any one of claims 1-10, wherein the one or more doses are independently administered to the subject at about 800 mg of the antibody or antigen-binding fragment.

16. The method of any one of claims 1-10, wherein the one or more doses are administered to the subject at about 1,600 mg of the antibody or antigen-binding fragment.

17. The method of any one of claims 1-16, wherein two or more doses of the antibody or antigen-binding fragment are administered to the subject.

18. The method of claim 17, wherein the two or more doses are administered to the subject at a frequency of between once a week and about once every four weeks.

19. The method of claim 17, wherein the two or more doses are administered to the subject at a frequency of about once a week.

20. The method of claim 17, wherein the two or more doses are administered to the subject at a frequency of about once every two weeks.

21. The method of claim 17, wherein the two or more doses are administered to the subject at a frequency of about once every three weeks.

22. The method of claim 17, wherein the two or more doses are administered to the subject at a frequency of about once every four weeks.

23. The method of any one of claims 1-18, wherein each dose comprises 800 mg of the antibody or antigen-binding fragment and is administered to the subject every 2 weeks.

24. The method of any one of claims 1-18, wherein each dose comprises 1600 mg of the antibody or antigen-binding fragment and is administered to the subject every 2 weeks.

25. The method of any one of claims 1-18, wherein individual doses of the antibody or antigen-binding fragment are administered to the subject on day 1 and day 15 of a 28-day cycle.

26. The method of claim 25, wherein the doses of the antibody or antigen-binding fragment are administered to the subject for multiple 28-day cycles.

27. The method of any one of claims 1-17, wherein the one or more doses comprise one or more induction doses that are administered to the subject during an induction phase and one or more maintenance doses that are administered to the subject during a maintenance phase after the one or more induction doses have been administered.

28. The method of claim 27, wherein one of the induction doses is administered to the subject about once a week for about 1-10 weeks.

29. The method of claim 27, wherein one of the induction doses is administered to the subject once a week for 8 weeks.

30. The method of claim 27, wherein one of the induction doses is administered 4 times within a 28-day cycle.

31. The method of claim 27, wherein one of the induction doses is administered 8 times within two 28-day cycles.

32. The method of claim 27, wherein one of the induction doses is independently administered on day 1, day 8, day 15 and day 22 for each of two 28-day cycles.

33. The method of any one of claims 27-32, wherein each induction dose comprises 100, 200, 400, 800, or 1600 mg of the antibody or antigen-binding fragment.

34. The method of claim 33, wherein each induction dose comprises 800 mg of the antibody or antigen-binding fragment.

35. The method of claim 33, wherein each induction dose comprises 1600 mg of the antibody or antigen-binding fragment.

36. The method of any one of claims 27-35, wherein the one or more maintenance doses are administered once every 1-4 weeks after completion of the induction phase.

37. The method of claim 36, wherein one of the maintenance doses is administered once every two weeks.

38. The method of claim 36, wherein one of the maintenance doses is administered on day 1 and day 15 of a 28-day cycle.

39. The method of any one of claims 36-38, wherein each maintenance dose comprises 100, 200, 400, 800, or 1600 mg of the antibody or antigen-binding fragment.

40. The method of claim 39, wherein each maintenance dose comprises 800 mg of the antibody or antigen-binding fragment.

41. The method of claim 39, wherein each maintenance dose comprises 1600 mg of the antibody or antigen-binding fragment.

42. The method of claim 27, wherein the antibody or antigen-binding fragment is dosed q1wk during the induction phase for a total of 8 induction phase doses and dosed q2wk during the maintenance phase.

43. The method of claim 27, wherein:
each induction dose comprises 100, 200, 400, or 1600 mg of the antibody or antigen-binding fragment;
each maintenance dose comprises 100, 200, 400, or 1600 mg of the antibody or antigen-binding fragment;
one of the induction doses is administered on each of day 1, day 8, day 15 and day 22 for each of two 28-day cycles for a total of 8 induction doses during the induction phase; and
one of the maintenance doses is administered on each of days 1 and day 15 of each of one or more subsequent cycles.

44. The method of claim 43, wherein each induction dose and each maintenance dose comprises 800 or 1600 mg of the antibody or antigen-binding fragment.

45. The method of claim 43, wherein each induction dose and each maintenance dose comprises 1600 mg of the antibody or antigen-binding fragment.

46. The method of any one of claims 1-45, wherein the method further comprises administering one or more doses of dexamethasone to the subject.

47. The method of claim 46, wherein the one or more doses of dexamethasone are independently administered to the subject at a frequency of once a week.

48. The method of claim 46, wherein one of the doses of the antibody or antigen-binding fragment are administered at a frequency of about once every 1-4 weeks and the doses of dexamethasone are administered at a frequency of about once every 1-4 weeks.

49. The method of claim 46, wherein one of the doses of the antibody or antigen binding fragment are administered once every two weeks and one of the doses of dexamethasone is administered once every two weeks.

50. The method of claim 46, wherein one of the doses of the antibody or antigen binding fragment is administered once every two weeks and one of the doses of dexamethasone is administered once every week.

51. The method of claim 46, wherein one of the doses of the antibody or antigen binding fragment is administered on each of day 1 and day 15 of a 28-day cycle and one of the doses of dexamethasone is administered on each of day 1, day 8, day 15 and day 22 of the same 28-day cycle.

52. The method of claim 46, wherein:
one of the doses of the antibody or antigen-binding fragment is administered once a week during an induction phase with subsequent doses following the induction phase being administered once every two weeks during a maintenance phase; and
one of the doses of dexamethasone is administered once every week.

53. The method of claim 52, wherein:
one of the doses of the antibody or antigen-binding fragment is administered once a week for 8 weeks during the induction phase and subsequent doses are administered once every two weeks during the maintenance phase; and
one of the doses of dexamethasone is administered once every week.

54. The method of claim 46, wherein:
one of the doses of the antibody or antigen-binding fragment is administered on each of day 1, day 8, day 15, and day 22 of each of two 28-day cycles and then on each of day 1 and day 15 of subsequent 28-day cycles; and
one of the doses of dexamethasone is administered on each of day 1, day 8, day 15, and day 22 of each of the 28-day cycles.

55. The method of any one of claims 46-54, wherein when the antibody or antigen-binding fragment are administered on the same day then dexamethasone is administered about 1-3 hours before the antibody or antigen binding fragment is administered.

56. The method of any one of claims 46-55, wherein each dose of the antibody or antigen-binding fragment is administered as a 800 mg dose.

57. The method of any one of claims 46-55, wherein each dose of the antibody or antigen-binding fragment is administered as a 1600 mg dose.

58. The method of any one of claims 46-55, wherein each dose of dexamethasone is administered as a 20-60 mg dose.

59. The method of claim 58, wherein each dose of dexamethasone is administered as a 40 mg dose.

60. The method of claim 58, wherein each dose of dexamethasone is administered as a 20 mg dose.

61. The method of any one of claims 46-60, wherein each dose of the antibody or antigen-binding fragment is administered as a 1600 mg dose, and wherein each dose of dexamethasone is administered as a 40 mg dose.

62. The method of any one of claims 1-61, wherein the method further comprises administering one or more doses of an anti-CD38 antibody, or antigen-binding fragment thereof to the subject.

63. The method of claim 62, wherein the anti-CD38 antibody is daratumumab.

64. The method of claim 62 or 63, wherein the one or more doses of the anti-CD38 antibody or antigen-binding fragment thereof are independently administered to the subject at about 5 mg/kg (milligram per kilogram of the body weight) to about 30 mg/kg.

65. The method of claim 64, wherein the one or more doses of the anti-CD38 antibody or antigen-binding fragment thereof are independently administered to the subject at about 10 mg/kg to about 20 mg/kg.

66. The method of claim 64, wherein the one or more doses of the anti-CD38 antibody or antigen-binding fragment thereof are independently administered to the subject at about 16 mg/kg.

67. The method of any one of claims 62-66, wherein two or more doses of the anti-CD38 antibody or antigen-binding fragment are administered to the subject.

68. The method of claim 67, wherein the two or more doses of the anti-CD38 antibody or antigen-binding fragment thereof are administered to the subject at a frequency of about once a week to about once every four weeks.

69. The method of claim 67, wherein the two or more doses of the anti-CD38 antibody or antigen-binding fragment thereof are administered to the subject at a frequency of about once a week.

70. The method of claim 67, wherein the two or more doses of the anti-CD38 antibody or antigen-binding fragment thereof are administered to the subject at a frequency of about once every two weeks or once every three weeks.

71. The method of claim 67, wherein the two or more doses of the anti-CD38 antibody or antigen-binding fragment thereof are administered to the subject at a frequency of about once every four weeks.

72. The method of claim 67, wherein the anti-CD38 antibody or antigen-binding fragment thereof are administered to the subject on day 1, day 8, day 15 and day 22 for 28-day cycles.

73. The method of claim 67, wherein two or more doses of the anti-CD38 antibody or antigen-binding fragment thereof are administered to the subject at a frequency of about once every week during a first phase; two or more doses of the anti-CD38 antibody or antigen-binding fragment thereof are administered to the subject at a frequency of about once every two weeks to about once every three weeks during a second phase; and two or more doses of the anti-CD38 antibody or antigen-binding fragment thereof are administered to the subject at a frequency of about once every four weeks during a third phase.

74. The method of claim 73, wherein the first phase is about 6 weeks to about 10 weeks.

75. The method of claim 74, wherein the first phase is about 8 weeks or about 9 weeks.

76. The method of any one of claims 73-75, wherein the second phase is about 10 weeks to about 20 weeks.

77. The method of any one of claims 73-76, wherein 8 doses of the anti-CD38 antibody or antigen-binding fragment thereof are administered to the subject at a frequency of about once every two weeks during the second phase.

78. The method of any one of claims 73-76, wherein 5 doses of the anti-CD38 antibody or antigen-binding fragment thereof are administered to the subject at a frequency of about once every three weeks during the second phase.

79. The method of any one of claims 73-78, wherein multiple doses of the anti-CD38 antibody or antigen-binding fragment thereof are administered to the subject at a frequency of about once every four weeks during the third phase until disease progression.

80. The method of any one of claims 1-79, wherein the method further comprises administering one or more doses of an immunomodulatory drug.

81. The method of claim 80, wherein the immunomodulatory drug is an immunomodulatory imide drug (IMiD).

82. The method of claim 81, wherein the immunomodulatory drug is lenalidomide or pomalidomide.

83. The method of 82, wherein the immunomodulatory drug is pomalidomide.

84. The method of any one of claims 80-83, wherein the one or more doses of the immunomodulatory drug are independently administered to the subject at a frequency of about once per day to about once per week.

85. The method of any one of claims 80-83, wherein the one or more doses of the immunomodulatory drug are independently administered to the subject once per day.

86. The method of any one of claims 80-83, wherein the one or more doses of the immunomodulatory drug are independently administered to the subject once per day on Days 1-21 of repeated 28-day cycles.

87. The method of any one of claims 80-86, wherein each dose of the immunomodulatory drug is about 1 mg to about 10 mg.

88. The method of any one of claims 80-86, wherein each dose of the immunomodulatory drug is about 2 mg to about 4 mg.

89. The method of any one of claims 80-86, wherein each dose of the immunomodulatory drug is about 4 mg.

90. The method of any one of claims 80-86, wherein when a dose of the immunomodulatory drug and a dose of the antibody or antigen-binding fragment that specifically binds to BCMA are administered on the same day, the dose of the immunomodulatory drug is administered about 1 to about 3 hours before the dose of the antibody or antigen-binding fragment that specifically binds to BCMA.

91. The method of any one of claims 80-83 and 86-90, wherein the antibody or antigen-binding fragment that specifically binds to BCMA is administered to the subject on day 1 and day 15 of a 28-day cycle, dexamethasone is administered to the subject on day 1, 8, 15, and 22 of the 28-day cycle, and pomalidomide is administered to the subject on days 1-21 of the 28-day cycle.

92. The method of any one of claims 80-83 and 86-90, wherein one of the induction doses of the antibody or antigen-binding fragment that specifically binds to BCMA is administered on each of day 1, day 8, day 15 and day 22 for two 28-day cycles for a total of 8 induction doses during the induction phase; and one of the maintenance doses of the antibody or antigen-binding fragment that specifically binds to BCMA is administered on each of days 1 and day 15 of each of one or more subsequent 28-day cycles in the maintenance phase;
wherein dexamethasone is administered to the subject on each of day 1, 8, 15, and 22 of each 28-day cycle in the induction phase and the maintenance phase; and
wherein pomalidomide is administered to the subject on days 1-21 of each 28-day cycle in the induction phase and the maintenance phase.

93. The method of claim 91 or 92, wherein when a dose of dexamethasone and a dose of the antibody or antigen-binding fragment are administered on the same day or a dose of pomalidomide and a dose of the antibody or antigen-binding fragment are administered on the same day, the dose of dexamethasone or the dose of pomalidomide is administered about 1 to about 3 hours before the dose of the antibody or antigen-binding fragment.

94. The method of any one of claims 1-93, wherein the method further comprises administering one or more doses of a gamma-secretase inhibitor to the subject.

95. The method of claim 94, wherein the gamma-secretase inhibitor is Semagacestat (LY450139), R04929097, MK-0752, Avagacestat (BMS-708163), BMS-986115, Nirogacestat (PF-03084014), Crenigacestat (LY3039478), BMS-906024, DAPT (GSI-IX), Dibenzazepine (YO-01027), LY411575, L-685,458, NGP 555, MDL-28170, or Itanapraced (CHF 5074).

96. The method of any one of claims 1-95, wherein each dose of the antibody or antigen-binding fragment is administered by systemic administration.

97. The method of claim 96, wherein the systemic administration is by intravenous administration.

98. The method of claim 96 or 97, wherein at least the initial dose of the antibody or antigen-binding fragment is administered to the subject using step-wise infusion.

99. The method of claim 98, wherein the step-wise infusion is performed using an infusion rate of about 50 mg/hour to about 400 mg/hour.

100. The method of claim 99, wherein, during the step-wise infusion, the infusion rate is increased every 30 minutes.

101. The method of claim 100, wherein, during the step-wise infusion, the infusion rate is increased no more than two-fold every 30 minute.

102. The method of any one of claims 1-101, wherein the subject is a human subject.

103. The method of claim 102, wherein the subject has previously been diagnosed as having multiple myeloma.

104. The method of any one of claims 1-103, wherein the subject has been diagnosed as having relapsed or refractory multiple myeloma.

105. The method of any one of claims 1-94, wherein the subject was previously administered one or more therapeutic agents or treatments for multiple myeloma.

106. The method of claim 105, wherein the previously administered one or more therapeutic agents or treatments for multiple myeloma were unsuccessful.

107. The method of claim 106, wherein the subject has previously been administered at least one of a proteasome inhibitor, an immunomodulatory agent, and an anti-CD38 antibody, or cannot tolerate the foregoing.

108. The method of claim 107, wherein the subject has previously been administered therapeutic agents include all three of a proteasome inhibitor, an immunomodulatory agent, and an anti-CD38 antibody, or cannot tolerate the foregoing.

109. The method of claim 106, wherein the subject has previously been administered at least 3 prior lines of anti-myeloma therapy and is refractory to at least one therapeutic agent in each of the following classes: a proteasome inhibitor, an immunomodulatory agent, and an anti-CD38 antibody.

110. The method of any one of claims 1-109, wherein the subject satisfies 1, 2 or all 3 of the following criteria prior to initiating treatment: serum monoclonal paraprotein (M-protein) level of ≥0.5 g/dL, urine M-protein level ≥200 mg/24 hr, and serum immunoglobulin free light chain ≥10 mg/dL and abnormal serum immunoglobulin kappa lambda free light chain ratio.

111. The method of any one of claims 1-110, wherein the method results in a steady-state concentration of the antibody, or antigen-binding fragment thereof, in the serum of the subject of about 1 μg/mL to about 200 μg/mL.

112. The method of any one of claims 1-111, wherein the method results in a steady-state concentration of free light chain (FLC) in the serum of the subject of less than 50 mg/dL.

113. The method of any one of claims 1-112, wherein the subject has received at least two prior lines of antimyeloma therapy and/or has documented IMWG (International Myeloma Working Group) disease progression on or within 60 days of completion of the two prior lines of antimyeloma therapy.

114. The method of any one of claims 1-113, wherein one or more therapeutic effects in the subject is improved after administration of the antibody-drug conjugate relative to a baseline.

115. The method of claim 114, wherein the one or more therapeutic effects is selected from the group consisting of: objective response rate, complete response rate, duration of response, duration of complete response, time to response, progression free survival, and overall survival.

116. The method of claim 115, wherein the objective response rate is at least about 20%, at least about 25%, at least about 30%, at least about 35%, at least about 40%, at least about 45%, at least about 50%, at least about 60%, at least about 70%, or at least about 80%.

117. The method of claim 115, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years.

118. The method of claim 115, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years.

119. The method of claim 115, wherein the duration of response or the duration of complete response to the treatment is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years.

120. A kit comprising:
(a) one or more doses of a pharmaceutical composition comprising (i) an antibody, or antigen binding fragment thereof, that specifically binds to a B cell maturation antigen (BCMA), and (ii) a pharmaceutically acceptable carrier, wherein the antibody or antigen binding fragment thereof, comprises: a heavy chain variable region comprising a CDR1 comprising SEQ ID NO: 1, a CDR2 comprising SEQ ID NO: 2, and a CDR3 comprising SEQ ID NO: 3, and a light chain variable domain comprising a CDR1 comprising SEQ ID NO: 5, a CDR2 comprising SEQ ID NO: 6, and a CDR3 comprising SEQ ID NO: 7; and optionally
(b) instructions for performing a method of any one of claims 1-119.

121. The kit of claim 120, wherein the kit further comprises one or more doses of dexamethasone, one or more doses of an immunomodulatory imide drug, one or more doses of a gamma-secretase inhibitor, and/or one or more doses of an anti-CD38 antibody or antigen-binding fragment thereof.

122. The kit of claim 121, wherein the kit further comprises one or more doses of dexamethasone.

123. The kit of claim 121, wherein the kit further comprises one or more doses of an immunomodulatory imide drug.

124. The kit of claim 121, wherein the kit further comprises one or more doses of dexamethasone and one or more doses of an immunomodulatory imide drug.

125. The kit of claim 121, wherein the kit further comprises one or more doses of a gamma-secretase inhibitor.

126. The kit of claim 121, wherein the kit further comprises one or more doses of an anti-CD38 antibody.
